SEQUENCE LISTING (2 sequences)

SEQ ID NO: 1 - protein, 1427 aa, Unknown
  Description of Unknown ADAMTS13 sequence
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHQEDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

SEQ ID NO: 2 - protein, 1427 aa, Artificial Sequence
  Description of Artificial Sequence Synthetic polypeptide
MHQRHPRARCPPLCVAGILACGFLLGCWGPSHFQQSCLQALEPQAVSSYLSPGAPLKGRPPSPGFQRQRQRQRRAAGGILHLELLVAVGPDVFQAHREDTERYVLTNLNIGAELLRDPSLGAQFRVHLVKMVILTEPEGAPNITANLTSSLLSVCGWSQTINPEDDTDPGHADLVLYITRFDLELPDGNRQVRGVTQLGGACSPTWSCLITEDTGFDLGVTIAHEIGHSFGLEHDGAPGSGCGPSGHVMASDGAAPRAGLAWSPCSRRQLLSLLSAGRARCVWDPPRPQPGSAGHPPDAQPGLYYSANEQCRVAFGPKAVACTFAREHLDMCQALSCHTDPLDQSSCSRLLVPLLDGTECGVEKWCSKGRCRSLVELTPIAAVHGRWSSWGPRSPCSRSCGGGVVTRRRQCNNPRPAFGGRACVGADLQAEMCNTQACEKTQLEFMSQQCARTDGQPLRSSPGGASFYHWGAAVPHSQGDALCRHMCRAIGESFIMKRGDSFLDGTRCMPSGPREDGTLSLCVSGSCRTFGCDGRMDSQQVWDRCQVCGGDNSTCSPRKGSFTAGRAREYVTFLTVTPNLTSVYIANHRPLFTHLAVRIGGRYVVAGKMSISPNTTYPSLLEDGRVEYRVALTEDRLPRLEEIRIWGPLQEDADIQVYRRYGEEYGNLTRPDITFTYFQPKPRQAWVWAAVRGPCSVSCGAGLRWVNYSCLDQARKELVETVQCQGSQQPPAWPEACVLEPCPPYWAVGDFGPCSASCGGGLRERPVRCVEAQGSLLKTLPPARCRAGAQQPAVALETCNPQPCPARWEVSEPSSCTSAGGAGLALENETCVPGADGLEAPVTEGPGSVDEKLPAPEPCVGMSCPPGWGHLDATSAGEKAPSPWGSIRTGAQAAHVWTPAAGSCSVSCGRGLMELRFLCMDSALRVPVQEELCGLASKPGSRREVCQAVPCPARWQYKLAACSVSCGRGVVRRILYCARAHGEDDGEEILLDTQCQGLPRPEPQEACSLEPCPPRWKVMSLGPCSASCGLGTARRSVACVQLDQGQDVEVDEAACAALVRPEASVPCLIADCTYRWHVGTWMECSVSCGDGIQRRRDTCLGPQAQAPVPADFCQHLPKPVTVRGCWAGPCVGQGTPSLVPHEEAAAPGRTTATPAGASLEWSQARGLLFSPAPQPRRLLPGPQENSVQSSACGRQHLEPTGTIDMRGPGQADCAVAIGRPLGEVVTLRVLESSLNCSAGDMLLLWGRLTWRKMCRKLLDMTFSSKTNTLVVRQRCGRPGGGVLLRYGSQLAPETFYRECDMQLFGPWGEIVSPSLSPATSNAGGCRLFINVAPHARIAIHALATNMGAGTEGANASYILIRDTHSLRTTAFHGQQVLYWESESSQAEMEFSEGFLKAQASLRGQYWTLQSWVPEMQDPQSWKGKEGT

(19) United States
(12) Patent Application Publication
(10) Pub. No.: US 2022/0249628 A1
(43) Pub. Date: Aug. 11, 2022

(54) USE OF RECOMBINANT ADAMTS13 FOR TREATING SICKLE CELL DISEASE

(71) Applicant: TAKEDA PHARMACEUTICAL COMPANY LIMIED, OSAKA (JP)

(72) Inventors: Paolo ROSSATO, Vienna (AT); Werner HOELLRIEGL, Altenmarkt/Triesting (AT); Friedrich SCHEIFLINGER, Vienna (AT)

(73) Assignee: TAKEDA PHARMACEUTICAL COMPANY LIMIED, OSAKA (JP)

(21) Appl. No.: 17/616,980

(22) PCT Filed: Jun. 5, 2020
(86) PCT No.: PCT/US20/36320

§ 371 Date: Dec. 6, 2021

Related U.S. Application Data
(60) Provisional application No. 62/858,691, filed on Jun. 7, 2019.
(60) Provisional application No. 63/004,389, filed on Apr. 2, 2020.

(51) Int. Cl. A61K 38/48; A61P 7/00

(52) U.S. Cl. CPC A61K 38/4886; C12Y 304/24087; A61P 7/00

(57) ABSTRACT

The disclosure provides a method for treating sickle cell disease with A Disintegrin And Metalloproteinase with Thrombospondin type 1 motif, member-13 (ADAMTS13). The disclosure provides a method for increasing ADAMTS13-mediated von Willebrand factor (VWF) cleavage in a subject suffering from sickle cell disease by administering ADAMTS13. The disclosure also provides a method of treating a vaso-occlusive crisis (VOC) in a subject suffering from sickle cell disease by administering ADAMTS13 after the onset of the VOC. The disclosure also provides a method of preventing a VOC in a subject suffering from sickle cell disease by administering ADAMTS13 prior to the onset of the VOC. The disclosure also provides a method of determining the efficacy of a treatment for a VOC in a mouse model.

### CROSS-REFERENCE TO RELATED APPLICATIONS

This application claims priority to U.S. Provisional Application Nos. 62/858,691, filed Jun. 7, 2019, and 63/004,389, filed Apr. 2, 2020, both of which are herein incorporated by reference in their entirety.

### FIELD OF THE INVENTION

The disclosure relates to a method for treating sickle cell disease with A Disintegrin And Metalloproteinase with Thrombospondin type 1 motif, member-13 (ADAMTS13). More particularly, the disclosure relates to a method for increasing ADAMTS13-mediated von Willebrand factor (VWF) cleavage in a subject suffering from sickle cell disease by administering ADAMTS13. The disclosure also relates to a method of treating a vaso-occlusive crisis (VOC) in a subject suffering from sickle cell disease by administering ADAMTS13 after the onset of the VOC. The disclosure also relates to a method of preventing a VOC in a subject suffering from sickle cell disease by administering ADAMTS13 prior to the onset of the VOC. The disclosure further relates to a method of determining the efficacy of a treatment for a VOC in a mouse model.

### BACKGROUND OF THE INVENTION

Sickle cell disease (SCD) is a worldwide distributed hereditary red blood cell disorder, which results from a point mutation (β^{s}, 6V) in the β-globin chain leading to the production of a defective form of hemoglobin, hemoglobin S (HbS). Studies of the kinetics of HbS polymerization following deoxygenation have shown it to be a high-order exponential function of hemoglobin concentration, thus highlighting a crucial role for cellular HbS concentration in sickling. Pathophysiological studies have shown that the dense, dehydrated red blood cells play a central role in acute and chronic clinical manifestations of SCD, in which intravascular sickling in capillaries, small vessels, and large vessels leads to vaso-occlusion and impaired blood flow with ischemic cell damage in a variety of organs and tissues.

Sickle cell disease patients with increased levels of von Willebrand factor (VWF) and high levels of ultra-large VWF multimers associated with acute vaso-occlusive events have been reported (Krishnan et al., Thromb Res; 122(4): 455-8, 2008; Kaul et al., Blood; 81(9): 2429-38, 1993). The levels of ultra-large VWF multimers are dependent on the activity of the metalloprotease A Disintegrin And Metalloproteinase with Thrombospondin type 1 motif, member-13 (ADAMTS13) that cleaves the hyperadhesive ultra-large VWF multimers under conditions of high fluid shear stress, playing an important role in maintaining a proper balance of hemostatic activity and thrombotic risk. ADAMTS13 cleaves VWF between residues Tyr1605 and Met1606, which corresponds to residues 842-843 after cleavage of the preprosequence, generating homodimers of 176 kDa and 140 kDa and smaller less platelet-adhesive VWF multimers (Furlan M, et al., Blood; 87(10): 4223-34, 1996; Tsai et al., Blood; 87(10): 4235-44, 1996; Crawley et al., Blood; 118(12): 3212-21, 2011). It is this ADAMTS13-mediated cleavage of VWF that is largely responsible for modulation of VWF multimeric size and hemostatic activity. Released VWF in circulating blood contributes to the formation of platelet thrombi as it binds to collagen and mediates platelet adhesion and agglutination in subendothelial tissues, including damaged vascular walls. VWF release is accompanied and partly triggered by activation of the vascular endothelium. Plasma of patients with SCD (both clinically asymptomatic and with acute painful crises) revealed very mild or no deficiency of ADAMTS13 activity compared to healthy individuals, but higher concentrations of VWF (particularly ULVWF multimers), and therefore a relative deficiency of ADAMTS13 to its substrate (Zhou et al., Curr Vasc Pharmacol; 10(6): 756-61, 2012; Schnog et al., Am J Hematol; 81: 492-8, 2006).

Sickling also causes the hemolysis of erythrocytes and consequently the release of excessive extracellular hemoglobin (ECHb). Increased ECHb in SCD patients inhibits ADAMTS13-mediated VWF proteolysis by binding to the A2 domain of VWF particularly to the ADAMTS13 cleavage site (Zhou et al., Anemia. 2011; 2011: 918916). Extracellular hemoglobin observed in SCD patients is usually at a concentration of 20-330 μg/mL in plasma, and >400 μg/mL during vaso-occlusive crises (Zhou et al., Thromb Haemost; 101(6): 1070-77, 2009). Thrombospondin-1 (TSP1), which is also increased in patients with SCD, binds to the A2 domain of ultra-large VWF multimers and also prevents VWF degradation by ADAMTS13 by competitively inhibiting ADAMTS13 activity.

SCD is a congenital, life-long illness. People with SCD inherit two abnormal hemoglobin β^{s} genes, one from each parent. When a person has two hemoglobin S genes, Hemoglobin SS (Hb SS), the disease is called sickle cell anemia. This is the most common and often most severe kind of SCD. Hemoglobin SC disease and hemoglobin SI thalassemia are two other common forms of SCD. In all forms of SCD, at least one of the two abnormal genes causes a person's body to make hemoglobin S or sickle hemoglobin, in their red blood cells. Hemoglobin is a protein in red blood cells that carries oxygen throughout the body. Sickle hemoglobin differs from normal hemoglobin in its propensity to form polymers under conditions of low oxygen tension, which form stiff rods within the red blood cell, changing it into a crescent, or sickle shape. Sickle-shaped cells are not flexible, which can cause a blockage that slows or stops the flow of blood and essentially obstructs the microcirculation. When this happens, oxygen cannot reach nearby tissues. The lack of tissue oxygen can cause attacks of sudden, severe pain, called vaso-occlusive crisis (VOC), pain crisis, or sickle cell crisis, which results in ischemic injury to the organ supplied and resultant pain. Pain crises constitute the most distinguishing clinical feature of VOC of SCD and are the leading cause of emergency department visits and hospitalizations for affected patients.

VOC is initiated and sustained by interactions among sickle cells, including sickle cell reticulocytes, endothelial cells, leukocytes, and plasma constituents, including VWF. Vaso-occlusion is responsible for a wide variety of clinical complications of SCD, including pain syndromes, stroke, leg ulcers, spontaneous abortion and renal insufficiency. The pain of VOC is often incompletely treated. Current treatment of VOC includes, among other things, the use of fluids, oxygen, and analgesia, while the incidence of VOC may be reduced with chronic red blood cell (RBC) transfusion as well as hydroxyurea. Despite advances in pain management, however, physicians are often reluctant to give patients adequate dosages of narcotic analgesics because of concerns about addiction, tolerance and side effects. In addition to acute VOC, other acute and chronic complications of SCD include renal disease, splenic infarction, increased risk of bacterial infection, acute and chronic anemia, chest syndrome, stroke and ocular disease.

Acute pain in patients with SCD is caused by ischemic tissue injury resulting from the occlusion of microvascular beds by sickled erythrocytes during an acute crisis. For example, the severe bone pain that is characteristic of VOC is believed to be caused by increased intra-medullary pressure, especially within the juxta-articular areas of long bones, secondary to an acute inflammatory response to vascular necrosis of the bone marrow by sickled erythrocytes. The pain may also occur because of involvement of the periosteum or periarticular soft tissue of the joints. The effect of unpredictable recurrences of acute crises on chronic pain creates a unique pain syndrome.

The severity of SCD varies widely from person to person. Advances in the diagnosis and care of SCD have extended the life expectancies of persons with SCD. In high-income countries like the United States, the life expectancy of a person with SCD is now about 40-60 years, whereas it was only 14 years about 40 years ago. At the present time, however, hematopoietic stem cell transplantation (HSCT) is the only cure for SCD. Unfortunately, most people with SCD are either too old for a transplant or do not have a relative who is a good enough genetic match for them to act as a donor for a successful transplant.

Further, clinical biomarkers for VOC in SCD are lacking. Therefore “time to readiness for discharge” and “time to discharge” are important components of the primary efficacy end point (time to resolution of VOC) (Telen M J, et al. Blood 2015; 125(17): 2656-2664, which is herein incorporated by reference in its entirety).

Thus, there is a need in the art for improved treatments of SCD, including the treatment of vaso-occlusive events of SCD that can reduce symptoms, prevent complications, and improve length and quality of life, as well as useful clinical biomarkers for VOC.

### SUMMARY OF THE INVENTION

In one aspect, the present disclosure provides a method for increasing A Disintegrin And Metalloproteinase with Thrombospondin type 1 motif, member-13 (ADAMTS13)-mediated VWF cleavage in a subject suffering from sickle cell disease, the method comprising administering to the subject in need thereof a therapeutically effective amount of a composition comprising ADAMTS13. In some embodiments, the ADAMTS13-mediated VWF cleavage in the subject is inhibited due to an increased plasma level of extracellular hemoglobin (ECHb) compared to a healthy subject. In some embodiments, the plasma level of extracellular hemoglobin (ECHb) in the subject is about 20-330 μg/mL. In some embodiments, the plasma level of extracellular hemoglobin (ECHb) in the subject is over 330 μg/mL.

In some embodiments, administering ADAMTS13 results in a reduction in the levels of at least one of ultra-large VWF multimers, VWF activity and VWF activity/antigen ratio compared to without ADAMTS13 treatment. In some embodiments, administering ADAMTS13 results in a reduction in the level of free hemoglobin in the plasma compared to without ADAMTS13 treatment.

In another aspect, the present disclosure provides a method for treating a vaso-occlusive crisis (VOC) in a subject suffering from sickle cell disease, the method comprising administering to the subject in need thereof a therapeutically effective amount of a composition comprising ADAMTS13 after the onset of the VOC.

In another aspect, the present disclosure provides a method for preventing a vaso-occlusive crisis (VOC) in a subject suffering from sickle cell disease, the method comprising administering to the subject in need thereof a therapeutically effective amount of a composition comprising ADAMTS13 prior to the onset of the VOC.

In some embodiments, the composition further comprises an ADAMTS13 variant. In some embodiments, the ADAMTS13 variant comprises an amino acid sequence with at least one single amino acid substitution as compared to the wildtype ADAMT13. In some embodiments, the wildtype ADAMTS13 is a human ADAMTS13. In some embodiments, the wildtype ADAMTS13 comprises the amino acid sequence of SEQ ID NO: 1. In some embodiments, at least one of the single amino acid substitutions is within the ADAMTS13 catalytic domain as compared to wildtype ADAMTS13. In some embodiments, the single amino acid substitution is not I⁷⁹M, V⁸⁸M, H⁹⁶D, R¹⁰²C, S¹¹⁹F, I¹⁷⁸T, R¹⁹³W, T¹⁹⁶I, S²⁰³P, L²³²Q, H²³⁴Q, D²³⁵H, A²⁵⁰V, S²⁶³C, and/or R²⁶⁸P as denoted in SEQ ID NO: 1, or the equivalent amino acid in an ADAMTS13. In some embodiments, the single amino acid substitution is at amino acid Q⁹⁷ as denoted in SEQ ID NO: 1, or the equivalent amino acid in an ADAMTS13. In some embodiments, the single amino acid change is from a Q to a D, E, K, H, L, N, P, or R. In some embodiments, the single amino acid change is from a Q to an R. In some embodiments, the ADAMTS13 variant comprises the amino acid sequence of SEQ ID NO: 2. In some embodiments, the ADAMTS13 variant consists essentially of SEQ ID NO: 2. In some embodiments, the ADAMTS13 variant consists of SEQ ID NO: 2.

In some embodiments of the therapeutic methods described herein, the therapeutically effective amount of ADAMTS13 and/or a variant thereof is from about 20 to about 6,000 international units per kilogram body weight. In some embodiments, the therapeutically effective amount of ADAMTS13 and/or a variant thereof is from about 300 to about 3,000 international units per kilogram body weight. In some embodiments, the therapeutically effective amount of ADAMTS13 and/or a variant thereof is from about 1000 to about 3,000 international units per kilogram body weight.

In some embodiments of the therapeutic methods described herein, administering the therapeutically effective amount of ADAMTS13 and/or a variant thereof results in a plasma concentration of ADAMTS13 and/or a variant thereof at about 1 to about 80 U/mL in the subject.

In some embodiments of the therapeutic methods described herein, the composition comprising ADAMTS13 and/or a variant thereof is administered in a single bolus injection, monthly, every two weeks, weekly, twice a week, daily, every 12 hours, every eight hours, every six hours, every four hours, or every two hours. In some embodiments, the composition comprising ADAMTS13 and/or a variant thereof is administered intravenously or subcutaneously.

In some embodiments of the therapeutic methods described herein, the ADAMTS13 and/or a variant thereof is recombinant. In some embodiments, the ADAMTS13 and/or a variant thereof is plasma derived. In some embodiments, the composition is in a stable aqueous solution ready for administration. In some embodiments, the therapeutically effective amount of the composition comprising ADAMTS13 and/or a variant thereof is sufficient to maintain an effective level of ADAMTS13 activity in the subject.

In some embodiments of the therapeutic methods described herein, the subject is a mammal. In some embodiments, the subject is a human.

In another aspect, the present disclosure provides a method of determining the efficacy of a treatment for a vaso-occlusive crisis (VOC) in a subject, the method comprising:
a) applying the treatment to the subject after the VOC;
b) collecting from the subject one or more behavioral symptoms selected from piloerection, apathy, eyes appearance, skin color, spontaneous mobility, stimulated mobility, and breathing frequency;
c) generating a score based on the severity of the one or more behavioral symptoms collected from step b);
d) comparing the score from step c) to a control score, wherein the control score is generated from a control subject that does not receive a treatment; and
e) (i) determining the treatment is effective if the score from step c) indicates less severity compared to the control score; (ii) determining the treatment is not effective if the score from step c) indicates more or the same severity compared to the control score.

In yet another aspect, the present disclosure provides a method of assessing the recovery of a subject from a vaso-occlusive crisis (VOC), the method comprising:
a) collecting from the subject one or more behavioral symptoms selected from piloerection, apathy, eyes appearance, skin color, spontaneous mobility, stimulated mobility, and breathing frequency after the VOC;
b) generating a score based on the severity of the one or more behavioral symptoms collected from step a);
c) comparing the score from step b) to a control score, wherein the control score is generated from the subject before the VOC or from a control subject that does not have a VOC; and
d) (i) determining the subject has recovered if the score from step b) indicates less or the same severity compared to the control score; (ii) determining the subject has not recovered if the score from step b) indicates more severity compared to the control score.

In some embodiments of the diagnostic methods described herein, the one or more behavioral symptoms are selected from piloerection, apathy, eyes appearance, stimulated mobility, and breathing frequency. In some embodiments, the behavioral symptoms are scored such that higher numbers are assigned to more severe symptoms.

In some embodiments of the diagnostic methods described herein, the subject is a mammal. In some embodiments, the subject is a mouse.

The foregoing summary is not intended to define every aspect of the invention, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the invention, are given by way of illustration only, because various changes and modifications within the spirit and scope of the invention will become apparent to those skilled in the art from this detailed description.

### DESCRIPTION OF THE FIGURES

FIG. 1 is an immunoblot of VWF cleavage fragments showing the inhibitory effect of increasing concentrations of hemoglobin. Cleavage reactions were performed at a constant concentration of ADAMTS13 (1 U/mL, 0.5 U/mL and 0.25 U/mL) in the presence of increasing concentrations of hemoglobin. The dimeric 176 kDa cleavage product is visualized by polyclonal anti-VWF antibody horseradish peroxidase (HRP) conjugate.

FIG. 2 shows the graphical evaluation of the inhibitory effect of increasing concentrations of hemoglobin.

FIG. 3 is an immunoblot of VWF cleavage fragments showing the overriding effect of rADAMTS13 concentration on the inhibitory effect of hemoglobin. Cleavage reactions were performed at a constant concentration of ADAMTS13 (0.25 U/mL, 0.5 U/mL, 1 U/mL and 2 U/mL) in the presence of increasing concentrations or in the absence of hemoglobin. The dimeric 176 kDa cleavage product is visualized by polyclonal anti-VWF antibody HRP conjugate.

FIG. 4 shows the graphical evaluation for the overriding effect of rADAMTS13 concentration on the inhibitory effect of hemoglobin.

FIG. 5 is an immunoblot of VWF cleavage fragments showing the evaluation of cleavage reaction with or without pre-incubation. +: with pre-incubation; c: hemoglobin free control; wo: without pre-incubation. Dimeric 176 kDa VWF fragment was visualized after incubation of VWF substrate with rADAMTS13 concentrations of 1 U/mL, 0.5 U/mL, and 0.25 U/mL in the presence of 0.5 mg/mL and lmg/mL hemoglobin with or without pre-incubation.

FIG. 6 shows graphical evaluation of ADAMTS13-mediated VWF multimer cleavage with and without pre-incubation with hemoglobin. wo: without.

FIGS. 7A-7C show ADAMTS13 activity versus time in Tim Townes SS mice dosed with 300 U/kg (FIG. 7A), 1000 U/kg (FIG. 7B), and 3000 U/kg SHP655 (FIG. 7C).

FIGS. 8A-8C show VWF activity/antigen ratio versus time in Tim Townes SS mice dosed with 300 U/kg (FIG. 8A), 1000 U/kg (FIG. 8B), and 3000 U/kg SHP655 (FIG. 8C).

FIGS. 9A-9C show plasma hemoglobin concentration versus time in Tim Townes SS mice dosed with 300 U/kg (FIG. 9A), 1000 U/kg (FIG. 9B), and 3000 U/kg SHP655 (FIG. 9C).

FIGS. 10A-10B are linear (FIG. 10A) and semi-logarithmic (FIG. 10B) plots showing the mean plasma concentration versus time profile for SHP655. The data shown in the plots are mean values with standard deviation.

FIG. 11 shows the survival curves of animals after five-hour exposure to 7.0% O₂ and one hour recovery at 21% O₂.

FIG. 12 shows the summary of behavioral scoring after five-hour exposure to 7.0% O₂ and one hour recovery at 21% O₂.

FIGS. 13A-13F show the single behavioral items including piloerection (FIG. 13A), eyes appearance (FIG. 13B), breathing (FIG. 13C), apathy (FIG. 13D), spontaneous activity (FIG. 13E), and stimulated activity (FIG. 13F) scored after five-hour exposure to 7.0% O₂ and one hour recovery at 21% O₂.

FIG. 14 shows the plasma level of free hemoglobin after five-hour exposure to 7.0% O₂ and one hour recovery at 21% O₂.

FIGS. 15A-15B show the ADAMTS13 activity (FIG. 15A) and antigen (FIG. 15B) level after five-hour exposure to 7.0% O₂ and one hour recovery at 21% O₂.

FIGS. 16A-16C show the VWF activity (FIG. 16A), antigen level (FIG. 16B) and activity normalized to antigen (FIG. 16C) after five-hour exposure to 7.0% O₂ and one hour recovery at 21% O₂.

FIGS. 17A-17B show semi-quantitative VWF multimer analysis of samples obtained after five-hour exposure to 7.0% O₂ and one hour recovery at 21% O₂.

FIGS. 18A-18C show the alignment between wildtype ADAMTS13 (SEQ ID NO: 1) and ADAMTS13 Q97R variant (SEQ ID NO: 2).

### DETAILED DESCRIPTION OF THE INVENTION

The disclosure provides, in various aspects, ADAMTS13 and related methods for preventing, ameliorating, and/or treating SCD, and particularly VOC in SCD. Before any embodiments of the disclosure are explained in detail, it is to be understood that the invention is not limited in its application to the details of construction and the arrangement of components set forth in the following description or illustrated in the figures and examples. The section headings used herein are for organizational purposes only and are not to be construed as limiting the subject matter described. All references cited in this application are expressly incorporated by reference herein for all purposes.

The disclosure embraces other embodiments and is practiced or carried out in various ways. Also, it is to be understood that the phraseology and terminology used herein is for the purpose of description and should not be regarded as limiting. The terms “including,” “comprising,” or “having” and variations thereof are meant to encompass the items listed thereafter and equivalents thereof as well as additional items.

The following abbreviations are used throughout.

AA mice Transgenic mice homozygous for Hemoglobin A (HbA)

ADAMTS A Disintegrin And Metalloproteinase with Thrombospondin

ADAMTS13 A Disintegrin And Metalloproteinase with Thrombospondin type 1 motif, member-13

BAL Bronchoalveolar lavage

DNA Deoxyribonucleic acid

ET-1 Endothelin 1

ECHb Extracellular hemoglobin

FRETS U FRETS units

GAPDH Glyceraldehyde 3-phosphate dehydrogenase

Hb Hemoglobin

HbA Hemoglobin A

HbS Sickle hemoglobin

HO-1 Heme-oxygenase 1

H/R Hypoxia/Reoxygenation

ICAM-1 Intercellular Adhesion Molecule 1

IU International Units

kDa KiloDalton

LDH Lactate dehydrogenase

NF-kB Nuclear Factor-kappa B

P-NF-kB Phospho-Nuclear Factor-kappa B

rADAMTS13 recombinant ADAMTS13

rVWF recombinant von Willebrand factor

RBC Red blood cell

RNA Ribonucleic acid

SCD Sickle Cell Disease

SS mice Transgenic mice homozygous for HbS

TXAS Thromboxane synthase

ULVWF ultra-large von Willebrand factor

VCAM-1 Vascular Cell Adhesion Molecule-1

VOC Vaso-occlusive crisis

VWF von Willebrand factor

It is noted here that, as used in this specification and the appended claims, the singular forms “a,” “an,” and “the” include plural reference unless the context clearly dictates otherwise. With respect to aspects of the disclosure described as a genus, all individual species are considered separate aspects of the disclosure. If aspects of the disclosure are described as “comprising” a feature, embodiments also are contemplated “consisting of” or “consisting essentially of” the feature.

As used herein, the following terms have the meanings ascribed to them unless specified otherwise.

The term “sickle cell disease (SCD),” as used herein, describes a group of inherited red blood cell disorders that exists in multiple forms. Some forms of SCD are Hemoglobin SS, Hemoglobin SC, Hemoglobin Sβ⁰ thalassemia, Hemoglobin Sβ⁺ thalassemia, Hemoglobin SD, and Hemoglobin SE. Although Hemoglobin SC disease and hemoglobin SI thalassemia are two common forms of SCD, the disclosure relates to and includes all forms of SCD.

The term “vaso-occlusive crisis (VOC),” as used herein, is an attack of sudden severe pain, which can occur without warning. VOC, also known as pain crisis or sickle cell crisis, is a common painful complication of SCD in adolescents and adults. VOC is initiated and sustained by interactions among sickle cells, endothelial cells and plasma constituents. Vaso-occlusion is responsible for a wide variety of clinical complications of SCD, including pain syndromes, stroke, leg ulcers, spontaneous abortion, and/or renal insufficiency.

“A disintegrin and metalloproteinase with a thrombospondin type 1 motif, member 13 (ADAMTS13)” is also known as von Willebrand factor-cleaving protease (VWFCP). The term “ADAMTS13” or “ADAMTS13 protein,” as used herein, includes ADAMTS13 analogs, variants, derivatives (including chemically-modified derivatives) and fragments thereof. In some aspects, the analogs, variants, derivatives, and fragments thereof have increased biological activity compared to ADAMTS13. In various aspects, ADAMTS13 is recombinant ADAMTS13 (rADAMTS13) or is blood-derived ADAMTS13, including plasma- and serum-derived ADAMTS13. In various embodiments of the present disclosure, ADAMTS13 is used interchangeably as SHP655 or BAX930 or TAK755.

In certain embodiments, the present disclosure includes variants of ADAMTS13. In certain embodiments, the ADAMTS13 variant comprises at least one single amino acid substitution as compared to the wildtype amino acid (e.g., SEQ ID NO: 1). In certain embodiments, the single amino acid substitution is within the catalytic domain of ADAMTS13 (e.g., amino acids 80 to 286 of SEQ ID NO: 1). In certain embodiments, the single amino acid substitution is at least one of I⁷⁹M, V⁸⁸M, H⁹⁶D, Q⁹⁷R, R¹⁰²C, S¹¹⁹F, I¹⁷⁸T, R¹⁹³W, T¹⁹⁶I, S²⁰³P, L²³²Q, H²³⁴Q, D²³⁵H, A²⁵⁰V, S²⁶³C, and/or R²⁶⁸P as denoted in SEQ ID NO: 1, or the equivalent amino acid in an ADAMTS13. In certain embodiments, the single amino acid substitution is not I⁷⁹M, V⁸⁸M, H⁹⁶D, R¹⁰²C, S¹¹⁹F, I¹⁷⁸T, R¹⁹³W, T¹⁹⁶I, S²⁰³P, L²³²Q, H²³⁴Q, D²³⁵H, A²⁵⁰V, S²⁶³C, and/or R²⁶⁸P as denoted in SEQ ID NO: 1, or the equivalent amino acid in an ADAMTS13. In certain embodiments, the ADAMTS13 variant comprises a single amino acid substitution at Q⁹⁷ as denoted in SEQ ID NO: 1, or the equivalent amino acid in an ADAMTS13. In certain embodiments, the amino acid change is from a Q to a D, E, K, H, L, N, P, or R. In certain embodiments, the amino acid change is from a Q to an R. In certain embodiments, the ADAMTS13 variant is ADAMTS13 Q⁹⁷R (SEQ ID NO: 2).

In certain embodiments, the present disclosure provides pharmaceutical compositions comprising at least one variant of ADAMTS13. In certain embodiments, the pharmaceutical composition comprises a combination of at least one ADAMTS13 variant and at least one wildtype ADAMTS13. In certain embodiments, the ratio of ADAMTS13 variant to wildtype ADAMTS13 is about 4:1 to about 1:4. In certain embodiments, the ratio of ADAMTS13 variant to ADAMTS13 wildtype is about 3:1. In certain embodiments, the ratio of ADAMTS13 variant to ADAMTS13 wildtype is about 1:1. In certain embodiments, the ratio of ADAMTS13 variant to ADAMTS13 wildtype is about 3:2. In certain embodiments, the ADAMTS13 variant comprises a single amino acid substitution at Q⁹⁷ as denoted in SEQ ID NO: 1, or the equivalent amino acid in an ADAMTS13. In certain embodiments, the ADAMTS13 variant is ADAMTS13 Q⁹⁷R (SEQ ID NO: 2). In certain embodiments, the wildtype ADAMTS13 is human ADAMTS13 or a biologically active derivative or fragment thereof as described in U.S. Patent Application Publication No. 2011/0229455, which is incorporated herein by reference for all purposes. In one embodiment, the amino acid sequence of hADAMTS13 is that of GenBank accession number NP_620594. In certain embodiments, the hADAMTS13 is SEQ ID NO: 1.

As used herein, an “analog” or “variant” refers to a polypeptide, e.g., ADAMTS13 variant, substantially similar in structure and having the same biological activity, albeit in certain instances to a differing degree, to a naturally-occurring molecule (e.g., SEQ ID NO: 1). Analogs or variants differ in the composition of their amino acid sequences compared to the naturally-occurring polypeptide from which the analog or variant is derived, based on one or more mutations involving (i) deletion of one or more amino acid residues at one or more termini of the polypeptide (including fragments as described above) and/or one or more internal regions of the naturally-occurring polypeptide sequence, (ii) insertion or addition of one or more amino acids at one or more termini (typically an “addition” analog or variant) of the polypeptide and/or one or more internal regions (typically an “insertion” analog or variant) of the naturally-occurring polypeptide sequence or (iii) substitution of one or more amino acids for other amino acids in the naturally-occurring polypeptide sequence. Substitutions are conservative or non-conservative based on the physico-chemical or functional relatedness of the amino acid that is being replaced and the amino acid replacing it. A “variant” includes the substitution, deletion, insertion, or modification of one or more amino acids in a peptide sequence, provided that the variant retains the biological activity of the native polypeptide. In some embodiments, a “variant” includes the substitution of one or more amino acid(s) with a similar or homologous amino acid(s) or a dissimilar amino acid(s). There are many scales on which amino acids can be ranked as similar or homologous. (Gunnar von Heijne, Sequence Analysis in Molecular Biology, p. 123-39 (Academic Press, New York, N.Y. 1987.). The term “variant,” in some aspects, is interchangeably used with the term “mutant”.

“Conservatively modified analogs” or “conservatively modified variants” applies to both amino acid and nucleic acid sequences. With respect to particular nucleic acid sequences, conservatively modified nucleic acids refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified analogs or variants. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, insertions, deletions, additions, or truncations to a nucleic acid, peptide, polypeptide, or protein sequence which alters, adds or deletes a single amino acid or a small percentage of amino acids in the encoded sequence is a “conservatively modified analog” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

8) Cysteine (C), Methionine (M) (see, e.g., Creighton, Proteins (1984)).

As used herein, an “allelic variant” refers to any of two or more polymorphic forms of a gene occupying the same genetic locus. Allelic variations arise naturally through mutation and, in some aspects, result in phenotypic polymorphism within populations. In certain aspects, gene mutations are silent (no change in the encoded polypeptide) or, in other aspects, encode polypeptides having altered amino acid sequences. “Allelic variants” also refer to cDNAs derived from mRNA transcripts of genetic allelic variants, as well as the proteins encoded by them.

The term “derivative” refers to polypeptides that are covalently modified by conjugation to therapeutic or diagnostic agents, labeling (e.g., with radionuclides or various enzymes), covalent polymer attachment such as pegylation (derivatization with polyethylene glycol) and insertion or substitution by chemical synthesis of non-natural amino acids. In some aspects, derivatives are modified to comprise additional chemical moieties not normally a part of the molecule. In certain aspects, these derivatives are called chemically-modified derivatives. Such moieties, in various aspects, modulate the molecule's solubility, absorption, and/or biological half-life. The moieties, in various other aspects, alternatively decrease the toxicity of the molecule and eliminate or attenuate any undesirable side effect of the molecule, etc. Moieties capable of mediating such effects are disclosed in Remington's Pharmaceutical Sciences (1980). Procedure for coupling such moieties to a molecule are well known in the art. For example, in some aspects, an ADAMTS13 derivative is an ADAMTS13 molecule having a chemical modification which confers a longer half-life in vivo to the protein. In one embodiment, the polypeptides are modified by addition of a water-soluble polymer known in the art. In a related embodiment, polypeptides are modified by glycosylation, PEGylation, and/or polysialylation.

As used herein, a “fragment” of a polypeptide refers to any portion of the polypeptide smaller than the full-length polypeptide or protein expression product. Fragments are typically deletion analogs of the full-length polypeptide, wherein one or more amino acid residues have been removed from the amino terminus and/or the carboxy terminus of the full-length polypeptide. Accordingly, “fragments” are a subset of deletion analogs described below.

The term “recombinant” or “recombinant expression system” when used with reference, e.g., to a cell, indicates that the cell has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, underexpressed or not expressed at all. This term also means host cells which have stably integrated a recombinant genetic element or elements having a regulatory role in gene expression, for example, promoters or enhancers. Recombinant expression systems as defined herein will express polypeptides or proteins endogenous to the cell upon induction of the regulatory elements linked to the endogenous DNA segment or gene to be expressed. The cells can be prokaryotic or eukaryotic.

The term “recombinant,” when used herein to refer to a polypeptide or protein, means that a polypeptide or protein is derived from recombinant (e.g., microbial or mammalian) expression systems. “Microbial” refers to recombinant polypeptides or proteins made in bacterial or fungal (e.g., yeast) expression systems. The term “recombinant variant” refers to any polypeptide differing from naturally occurring polypeptides by amino acid insertions, deletions, and substitutions, created using recombinant DNA techniques. Guidance in determining which amino acid residues may be replaced, added or deleted without abolishing activities of interest may be found by comparing the sequence of the particular polypeptide with that of homologous peptides and minimizing the number of amino acid sequence changes made in regions of high homology.

The term “agent” or “compound” describes any molecule, e.g., protein or pharmaceutical, with the capability of affecting a biological parameter in the disclosure.

A “control,” as used herein, can refer to an active, positive, negative or vehicle control. As will be understood by those of skill in the art, controls are used to establish the relevance of experimental results, and provide a comparison for the condition being tested. In certain aspects, a control is a subject that does not receive an active prophylactic or therapeutic composition. In certain aspects, a control is a subject not experiencing SCD, and/or VOC, for example, but not limited to a healthy control or a subject without any symptoms.

The term “reduces the severity,” when referring to a symptom of SCD, and/or VOC in SCD, means that the symptom has delayed onset, reduced severity, reduced frequency, or causes less damage to the subject. Generally, severity of a symptom is compared to a control, e.g., a subject that does not receive an active prophylactic or therapeutic composition, or as compared to the severity of the symptom prior to administration of the therapeutic. In that case, a composition can be said to reduce the severity of a symptom of SCD, and/or VOC in SCD, if the symptom is reduced by about 10%, about 15%, about 20%, about 25%, about 30%, about 35%, about 40%, about 50%, about 60%, about 70%, about 80%, about 90%, or about 100% (i.e., essentially eliminated), as compared to the control level of the symptom. In certain aspects, a composition can be said to reduce the severity of a symptom of SCD and/or VOC in SCD if the symptom is reduced between about 10% to about 100%, about 20% to about 90%, about 30% to about 80%, about 40% to about 70% or about 50% to about 60%, as compared to the control level of the symptom. In certain aspects, a composition can be said to reduce the severity of a symptom of SCD and/or VOC in SCD if the symptom is reduced between about 10% to about 30%, about 20% to about 40%, about 30% to about 50%, about 40% to about 60%, about 50% to about 70%, about 60% to about 80%, about 70% to about 90% or about 80% to about 100%, as compared to the control level of the symptom. In some aspects, treatment by methods of the disclosure reduces the severity of the pain and/or other symptoms of VOC in SCD.

The terms “reduces the expression,” “reduces the level,” and “reduces the activation” when referring to a biomarker of SCD and/or VOC in SCD (for example, but not limited to ultra-large VWF multimers, VWF activity and VWF activity/antigen ratio, ECHb VCAM-1, ICAM-1, P-NF-kB/NF-kB ratio, ET-1, TXAS, HO-1, Hct, Hb, MCV, HDW, reticulocyte numbers, and neutrophil numbers), means that the expression, level, and/or activation of a biomarker has been reduced as compared to control. In that case, a composition can be said to reduce the expression, level, and/or activation of a biomarker of SCD and/or VOC in SCD if the biomarker is reduced by about 10%, about 15%, about 20%, about 25%, about 30%, about 35%, about 40%, about 50%, about 60%, about 70%, about 80%, about 90%, or about 100% (i.e., essentially eliminated), as compared to the control. In certain aspects, a composition can be said to reduce the expression, level, and/or activation of SCD and/or VOC in SCD if the expression, level, and/or activation is reduced between about 10% to about 100%, about 20% to about 90%, about 30% to about 80%, about 40% to about 70% or about 50% to about 60%, as compared to the control. In certain aspects, a composition can be said to reduce the expression, level, and/or activation of a biomarker of SCD and/or VOC in SCD if the biomarker is reduced between about 10% to about 30%, about 20% to about 40%, about 30% to about 50%, about 40% to about 60%, about 50% to about 70%, about 60% to about 80%, about 70% to about 90% or about 80% to about 100%, as compared to the control.

The terms “increases the expression,” “increases the level,” and “increases the activation” when referring to a biomarker of SCD and/or VOC in SCD, means that the expression, level, and/or activation of a biomarker has been increased as compared to control. In that case, a composition can be said to increase the expression, level, and/or activation of a biomarker of SCD and/or VOC in SCD if the biomarker is increased by about 10%, about 15%, about 20%, about 25%, about 30%, about 35%, about 40%, about 50%, about 60%, about 70%, about 80%, about 90%, or about 100% (i.e., essentially eliminated), as compared to the control. In certain aspects, a composition can be said to increase the expression, level, and/or activation of SCD and/or VOC in SCD if the expression, level, and/or activation is increased between about 10% to about 100%, about 20% to about 90%, about 30% to about 80%, about 40% to about 70% or about 50% to about 60%, as compared to the control. In certain aspects, a composition can be said to increase the expression, level, and/or activation of a biomarker of SCD and/or VOC in SCD if the biomarker is increased between about 10% to about 30%, about 20% to about 40%, about 30% to about 50%, about 40% to about 60%, about 50% to about 70%, about 60% to about 80%, about 70% to about 90% or about 80% to about 100%, as compared to the control.

The terms “effective amount” and “therapeutically effective amount” each refer to the amount of polypeptide, e.g., ADAMTS13 polypeptide, or composition used to support an observable level of one or more biological activities of the ADAMTS13 polypeptide, as set forth herein. For example, an effective amount, in some aspects of the disclosure, would be the amount necessary to treat or prevent symptoms of VOC in SCD.

A “subject” is given its conventional meaning of a non-plant, non-protist living being. In most aspects, the subject is an animal. In particular aspects, the animal is a mammal. In more particular aspects, the mammal is a human. In other aspects, the mammal is a pet or companion animal, a domesticated farm animal, or a zoo animal. In certain aspects, the mammal is a mouse, rat, rabbit, guinea pig, pig, or non-human primate. In particular aspects, the animal is a mouse. In other aspects the mammal is a cat, dog, horse, or cow. In various other aspects, the mammal is a deer, mouse, chipmunk, squirrel, opossum, or raccoon.

It also is specifically understood that any numerical value recited herein includes all values from the lower value to the upper value, i.e., all possible combinations of numerical values between the lowest value and the highest value enumerated are to be considered to be expressly stated in this application. For example, if a concentration range is stated as about 1% to 50%, it is intended that values such as 2% to 40%, 10% to 30%, or 1% to 3%, etc., are expressly enumerated in this specification. The values listed above are only examples of what is specifically intended.

Ranges, in various aspects, are expressed herein as from “about” or “approximately” one particular value and/or to “about” or “approximately” another particular value. When values are expressed as approximations, by use of the antecedent “about,” it will be understood that some amount of variation is included in the range. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

### Sickle Cell Disease and Vaso-Occlusion in Sickle Cell Disease

In some aspects, the disclosure includes ADAMTS13 and compositions comprising ADAMTS13 in the treatment, amelioration, and/or prevention of SCD, and particularly VOC in SCD. SCD is a worldwide hereditary red blood cell disorder caused by a point mutation in the β-globin gene resulting in the synthesis of pathological HbS, and abnormal HbS polymerization in hypoxic conditions. The two main clinical manifestations of SCD are chronic hemolytic anemia and acute VOC, which are the principal causes of hospitalization of SCD patients. Recent studies have underscored the central role of sickle vasculopathy in the generation of sickle cell-related acute events and chronic organ complications (Sparkenbaugh et al., Br. J. Haematol. 162:3-14, 2013; De Franceschi et al., Semin. Thromb. Hemost. 226-36, 2011; and Hebbel et al., Cardiovasc. Hematol. Disord. Drug Targets, 9:271-92, 2009; Dutra et al., Proc Natl Acad Sci USA; 111(39): E4110-E4118; each of which is herein incorporated by reference in its entirety). The pathophysiology of these complications is based on intravascular sickling in capillaries and small vessels leading to VOC, impaired blood flow, vascular inflammation, and/or thrombosis with ischemic cell damage.

The most common clinical manifestation of SCD is VOC. A VOC occurs when the microcirculation is obstructed by sickled red blood cells, causing ischemic injury to the organ supplied and resultant pain. Pain crises constitute the most distinguishing clinical feature of SCD and are the leading cause of emergency department visits and/or hospitalizations for affected SCD subjects or patients.

Approximately half the SCD subjects or patients with homozygous HbS disease experience VOC. The frequency of crisis is extremely variable. Some SCD subjects or patients have as many as six or more episodes annually, whereas others may have episodes only at great intervals or none at all. Each subjects or patient typically has a consistent pattern for crisis frequency.

The disclosure includes methods for reducing at least one symptom of VOC including, but not limited to, ischemia and pain (e.g., dactylitis, priapism, abdominal, chest, and joint), jaundice, bone infarction, abnormal breathing (e.g., tachypnea and shortness of breath), hypoxia, acidosis, hypotension, and/or tachycardia associated with VOC. In certain aspects, VOC can be defined as a condition comprising one or more of these symptoms. Pain crises begin suddenly. The crisis may last several hours to several days and terminate as abruptly as it began. The pain can affect any body part and often involves the abdomen, appendages, chest, back, bones, joints, and soft tissue, and it may present as dactylitis (bilateral painful and swollen hands and/or feet in children), acute joint necrosis or avascular necrosis, or acute abdomen. With repeated episodes in the spleen, infarctions and autosplenectomy predisposing to life-threatening infection are usual. The liver also may infarct and progress to failure with time. Papillary necrosis is a common renal manifestation of VOC, leading to isosthenuria (i.e., inability to concentrate urine).

Severe deep pain is present in the extremities, involving long bones. Abdominal pain can be severe, resembling acute abdomen; it may result from referred pain from other sites or intra-abdominal solid organ or soft tissue infarction. Reactive ileus leads to intestinal distention and pain. The face also may be involved. Pain may be accompanied by fever, malaise, trouble breathing, painful erections, jaundice and leukocytosis. Bone pain is often due to bone marrow infarction. Certain patterns are predictable, as pain tends to involve bones with the most bone marrow activity and because marrow activity changes with age. During the first 18 months of life, the metatarsals and metacarpals can be involved, presenting as dactylitis or hand-foot syndrome. Although the above patterns describe commonly encountered presentations, any area of the body of the subject with blood supply and sensory nerves can be affected in VOC.

Often, no precipitating cause can be identified for what causes a VOC. However, because deoxygenated HbS becomes semi-solid, the most likely physiologic trigger of VOC is hypoxemia. This may be due to acute chest syndrome or accompany respiratory complications. Dehydration also can precipitate pain, since acidosis results in a shift of the oxygen dissociation curve (Bohr effect), causing hemoglobin to desaturate more readily. Hemoconcentration also is a common mechanism. Another common trigger of VOC are changes in body temperature, whether an increase due to fever or a decrease due to environmental temperature change. Lowered body temperature likely leads to crises as the result of peripheral vasoconstriction.

In certain embodiments, VOC can be defined as having an increase in peripheral neutrophils as compared to a control. In certain embodiments, VOC can be defined as an increase in pulmonary vascular leakage (e.g., increased number of leukocytes in a bronchoalveolar lavage (BAL) and/or protein content (BAL protein (mg/mL)) as compared to a control.

In certain embodiments, increased levels of vascular activation (e.g., as measured by increased expression, levels, and/or activity of VCAM-1 and/or ICAM-1) in an organ, as compared to control, is a marker for VOC. In certain embodiments, increased levels of inflammatory vasculopathy (e.g., as measured by increased expression, levels, and/or activity of VCAM-1 and/or ICAM-1) in an organ, as compared to control, is a marker for VOC. In certain embodiments, increased levels of vascular activation and inflammatory vasculopathy in a tissue, as compared to control, is a marker for VOC. In certain embodiments, the organ is lung and/or kidney. In certain embodiments, the organ is kidney.

In certain embodiments, VOC can be defined as the increased expression, levels, and/or activation of at least one of NF-kB (wherein activation of NF-kB is measured by P-NF-kB or the ratio of P-NF-kB/NF-kB), VCAM-1 and ICAM-1 as compared to control. In certain embodiments, VOC can be defined as increased expression or level of at least one of endothelin-1 (ET-1), thromboxane synthase (TXAS), and heme-oxygenase-1 (HO-1) as compared to control. In certain embodiments, these increases are seen in lung tissue. In certain embodiments, these increases are seen in kidney tissue. In certain embodiments, increased expression and/or levels of TXAS, ET-1, and VCAM-1, and activation of NF-kB in the kidney tissue are markers for VOC.

In certain embodiments, VOC can be defined by hematology parameters. In certain embodiments, VOC can be defined as a decrease in the levels of at least one of Hct, Hb, MCV, and MCH as compared to control. In certain embodiments, VOC can be defined as a decrease in the levels of at least two of Hct, Hb, MCV, and MCH as compared to control. In certain embodiments, VOC can be defined as a decrease in the levels of at least three of Hct, Hb, MCV, and MCH as compared to control. In certain embodiments, VOC can be defined as an increase in the levels of at least one of CHCM, HDW, neutrophil numbers, and LDH as compared to control. In certain embodiments, VOC can be defined as an increase in the levels of at least two of CHCM, HDW, neutrophil numbers, and LDH as compared to control. In certain embodiments, VOC can be defined as an increase in the levels of at least three of CHCM, HDW, neutrophil numbers, and LDH as compared to control. In certain embodiments, VOC can be defined as a decrease in Hct levels as compared to control. In certain embodiments, VOC can be defined as a decrease in Hb levels as compared to control. In certain embodiments, VOC can be defined as a decrease in MCV as compared to control. In certain embodiments, VOC can be defined as a decrease in MCH as compared to control. In certain embodiments, VOC can be defined as an increase in CHCM as compared to control. In certain embodiments, VOC can be defined as an increase in HDW as compared to control. In certain embodiments, VOC can be defined as an increase in neutrophil numbers as compared to control. In certain embodiments, VOC can be defined as an increase in LDH as compared to control. In certain embodiments, VOC can be defined as a decrease in the levels of at least one of Hct, Hb, MCV, and MCH as compared to control and/or an increase in the levels of at least one of CHCM, HDW, neutrophil numbers, and LDH as compared to control. In certain embodiments, VOC can be defined as a decrease in the levels of Hct, Hb, MCV, and MCH as compared to control and/or an increase in the levels of CHCM, HDW, neutrophil numbers, and LDH as compared to control.

### Models of SCD and Methods of Testing Effectiveness of Prophylaxis or Treatment

In some embodiments, the disclosure includes study of the effects of a recombinant ADAMTS13 (i.e., BAX930/SHP655/TAK755) in a mouse model of SCD (Tim Townes mouse) during acute SCD related events, mimicked by exposing SCD mice to hypoxia. Studies are carried out under normoxic and hypoxic conditions, wherein efficacy of the prophylaxis or treatment dose(s) in the mouse model (including measuring overall survival) and biological effects of the treatment(s) with BAX930/SHP655/TAK755 on blood parameters. lung and kidney injury and vascular inflammation are studied after exposing sickle cell disease mice to hypoxia.

The humanized Tim Townes SS mice are published as an appropriate mouse model for SCD. It is a transgenic mouse model, with a knock-out for the genes of murine hemoglobin and knock-in for the genes of human hemoglobin S (HbS, called SCD mice or SS mice) (Ryan et al., Science; 278(5339): 873-6, 1997; Nguyen et al., Blood, 124(21): 4916, 2014). A single IV dose treatment of Tim Townes SS mice with a high dose (2940 U/kg) of ADAMTS13 significantly reduced the severity of vaso-occlusive events and these mice survived under hypoxia conditions compared to control animals (see Example 7 of International Publication No. WO/2018/027169, which is incorporated herein by reference in its entirety).

In some embodiments, a transgenic mouse model of SCD is used (Kalish et al., Haematologica 100:870-80, 2015). In some aspects, healthy control (Hba^{tm1(HBA)Tow} Hbb^{tm3(HBG1,HBB)Tow}) and SCD (Hba^{tm1(HBA)Tow} Hbb^{tm2(HBG1,HBB*)Tow}) mice are exposed to hypoxia/re-oxygenation (H/R) stress (Kalish et al., infra). Such H/R stress has been shown to biologically recapitulate the acute VOC and organ damage observed in acute VOC in human SCD patients. In some aspects, healthy (AA) and SCD (SS) mice are subjected to hypoxia (e.g., about 5.5 or 7% oxygen) for certain time periods (e.g., about 5 hours) followed by certain time periods (e.g., 1 hours) of re-oxygenation (e.g., about 21% oxygen, room air condition).

In various aspects, models of SCD and controls are subject to conditions of normoxia or hypoxia. In normoxia experiments, healthy control (AA) and SCD (SS) mice receive a single intravenous administration of either rADAMTS13 (e.g., 3,000 IU/kg)) or buffer (vehicle) at a fixed volume (e.g., 10 mL/kg) and are subject to normoxic (e.g., about 21% oxygen, room air condition) conditions. Animals are studied for varied periods of time after treatment with ADAMTS13 or vehicle and exposure to normoxia or hypoxia. Blood is collected and complete blood count (CBC) is measured. A CBC is a blood test used to evaluate overall health and detect a wide range of disorders, including among other things, anemia. Various other endpoints, including but not limited to, hematology, coagulation parameters, biomarkers of inflammation, vasculopathy, and histopathology are measured.

In exemplary aspects, hypoxia experiments are carried out, wherein healthy control (AA) and SCD (SS) mice receive a single intravenous administration of ADAMTS13 (e.g., 300 IU/kg, 1,000 IU/kg or 3,000 IU/kg) or vehicle at an affixed volume (e.g., 10 mL/kg). In certain embodiments, the dose administered to a human subject is about 10% that administered to a rodent (e.g., mouse) subject. In certain embodiments, the dose administered to a human subject is about 9% that administered to a rodent (e.g., mouse) subject. In certain embodiments, the dose administered to a human subject is about 8% that administered to a rodent (e.g., mouse) subject. In certain embodiments, the dose administered to a human subject is about 7% that administered to a rodent (e.g., mouse) subject. In certain embodiments, the dose administered to a human subject is less than about 10%, e.g., about 7% to about 10%, that administered to a rodent (e.g., mouse) subject.

After injection (e.g., about 1-3 hours after injection), mice are exposed to hypoxia (e.g., about 7% oxygen) for a time period (e.g., about 5 hours) followed by a time period of re-oxygenation (e.g., about 1 hours) to mimic SCD related VOC events. In some aspects, the same parameters as detailed for normoxic studies are evaluated.

In additional exemplary aspects, hypoxia experiments are carried out, wherein healthy control (AA) and SCD (SS) mice are exposed to hypoxia (e.g., about 8% oxygen, or higher) for a time period (e.g., about 10 hours) followed by a time period of re-oxygenation (e.g., about 3 hours) to mimic SCD related VOC events. Then, at various time points thereafter including, but not limited to, immediately after, or about 1, 3, 6, 12, 24, 36, 48 or 72 hours after the experimentally-induced vaso-inclusive event, mice receive either a single intravenous administration of ADAMTS13 (e.g., 300 IU/kg, 1,000 IU/kg or 3,000 IU/kg) or vehicle at an affixed volume (e.g., 10 mL/kg), or multiple injections at 12 or 24 intervals. In some aspects, the same parameters as detailed for normoxic studies are evaluated.

In various aspects, any target tissue is examined for effectiveness of treatment with ADAMTS13 in in vitro or in vivo models and/or under conditions of VOC. In some aspects, organ tissue includes, but is not limited to, lung, liver, pancreas, skin, retina, prostate, ovary, lymph node, adrenal gland, kidney, heart, gallbladder or GI tract. In some aspects, organ tissue includes, but is not limited to the lungs, liver, spleen, and/or kidneys.

For example, in some aspects, target tissues are collected to examine effects of ADAMTS13 under conditions of normoxia or hypoxia. Tissues are frozen and/or fixed in formalin. Frozen tissues are used for immunoblot analysis with specific antibodies against nuclear factor-kappa B (NF-kB), endothelin-1 (ET-1), heme-oxygenase 1 (HO-1), intercellular adhesion molecule-1 (ICAM-1), thromboxane synthase (TXAS), and vascular cell adhesion molecule-1 (VCAM-1). Fixed organs are used for standard pathology (H&E staining).

Markers of vaso-constriction, platelet aggregation, inflammation, oxidative stress, anti-oxidant response and/or tissue damage may be measured to determine effectiveness of treatment. In some aspects, nuclear factor kappa B is measured in both its normal (NF-kB) and activated (P-NF-kB) forms. NF-kB is a transcriptional factor which has been described to coordinate the inflammatory and anti-oxidant response. The ratio between the activated and the normal forms is evaluated. In some aspects, ET-1 is measured. ET-1 is a potent vasoconstrictor that is produced by vascular endothelial cells. ET-1 plays a role in several pathophysiological processes, including cardiovascular hypertrophy, pulmonary hypertension and chronic renal failure. In some aspects, HO-1 is measured. HO-1 is the inducible, rate-limiting enzyme in the catabolism of heme and might attenuate the severity of outcomes from vaso-occlusive and hemolytic crises, acting as a vaso-protective anti-oxidant. In some aspects, ICAM-1 is measured. ICAM-1 is continuously present in low concentrations in the membranes of leukocytes and endothelial cells. Although ICAM-1 does not appear to be involved in sickle cell adhesion to vascular endothelium, ICAM-1 may exacerbate VOC by promoting leukocyte adhesion. In some aspects, TXAS is measured. TXAS is an endoplasmic reticulum membrane protein that catalyzes the conversion of prostaglandin H2 to thromboxane A2. TXAS is a potent vasoconstrictor and inducer of platelet aggregation. Thus, TXAS is a potent inducer of vaso-constriction and platelet aggregation. TXAS plays a role in several pathophysiological processes including hemostasis, cardiovascular disease, and stroke. In some aspects, VCAM-1 is measured. VCAM-1 mediates the adhesion of lymphocytes and other blood cells to the vascular endothelium and therefore may contribute to vaso-occlusive events. In some aspects, inflammatory cell infiltrates are measured in organ tissue.

In exemplary aspects, immunoblot analyses with specific antibodies against NF-kB, ET-1, HO-1, ICAM-1, TXAS, and VCAM-1 are carried out to measure the expression of these enzymes in the cells and tissues of models or subjects of the disclosure to determine effectiveness of treatment. In exemplary aspects, the expression of NF-kB, ET-1, HO-1, ICAM-1, TXAS, and/or VCAM-1 is measured in organ tissue from AA and SCD mice treated with either vehicle or ADAMTS13. In certain embodiments, organs include, but are not limited to, lung, liver, pancreas, skin, retina, prostate, ovary, lymph node, adrenal gland, kidney, heart, gallbladder or GI track. In certain embodiments, the organ is lung, liver, spleen, and/or kidney.

In certain embodiments, administration of ADAMTS13 results in reduced levels of vascular activation and/or inflammatory vasculopathy in an organ as compared to control. In certain embodiments, the organ is lung. In certain embodiments, the organ is kidney.

In certain embodiments, administration of ADAMTS13 results in reduced expression, level, and/or activation of at least one of VCAM-1, ICAM-1, NF-kB (wherein reduced activation of NF-kB is measured by P-NF-kB or the ratio of P-NF-kB/NF-kB), ET-1, TXAS, and HO-1 as compared to control. In certain embodiments, administration of ADAMTS13 results in reduced expression, level, and/or activation of at least two of VCAM-1, ICAM-1, NF-kB, ET-1, TXAS, and HO-1 as compared to control. In certain embodiments, administration of ADAMTS13 results in reduced expression, level, and/or activation of at least three of VCAM-1, ICAM-1, NF-kB, ET-1, TXAS, and HO-1 as compared to control. In certain embodiments, administration of ADAMTS13 results in reduced expression, level, and/or activation of at least four of VCAM-1, ICAM-1, NF-kB, ET-1, TXAS, and HO-1 as compared to control. In certain embodiments, administration of ADAMTS13 results in reduced expression, level, and/or activation of at least five of VCAM-1, ICAM-1, NF-kB, ET-1, TXAS, and HO-1 as compared to control. In certain embodiments, administration of ADAMTS13 results in reduced expression, level, and/or activation of VCAM-1, ICAM-1, NF-kB, ET-1, TXAS, and HO-1 as compared to control. In certain embodiments, administration of ADAMTS13 results in reduced expression, level, and/or activation of VCAM-1 as compared to control. In certain embodiments, administration of ADAMTS13 results in reduced expression, level, and/or activation of ICAM-1 as compared to control. In certain embodiments, administration of ADAMTS13 results in reduced expression, level, and/or activation of VCAM-1 and ICAM-1 as compared to control. In certain embodiments, administration of ADAMTS13 results in reduced expression and/or level of ET-1 as compared to control. In certain embodiments, administration of ADAMTS13 results in reduced expression and/or level of TXAS as compared to control. In certain embodiments, administration of ADAMTS13 results in reduced expression and/or level of HO-1 as compared to control. In certain embodiments, administration of ADAMTS13 results in reduced ratio of P-NF-kB/NF-kB as compared to control. In certain embodiments, administration of ADAMTS13 results in a reduction of at least one of P-NF-kB/NF-kB ratio, ET-1 expression and/or level, TXAS expression and/or level, and HO-1 expression and/or level as compared to control. In certain embodiments, administration of ADAMTS13 results in a reduction of P-NF-kB/NF-kB ratio, ET-1 expression and/or level, TXAS expression and/or level, and HO-1 expression and/or level as compared to control. In certain embodiments, the organ is lung. In certain embodiments, the organ is kidney.

In further exemplary aspects, the measurement of these markers is carried out after the animal models are subject to conditions of hypoxia and reoxygenation (H/R) as described herein. In further exemplary aspects, the measurement of these markers is carried out after the subjects experience VOC.

In some embodiments, blood flow is measured as an indicatory of treatment effectiveness. In some embodiments, blood flow is measured by, but not limited to, ultrasound, PET, fMRI, NMR, laser Doppler, electromagnetic blood flow meter, or a wearable device.

In some embodiments, reduction or prevention of thrombosis is a measurement of the effectiveness of the treatment. In some embodiments, the presence of thrombosis is measured by, but not limited to, histopathological examination, ultrasound, D-dimer test, venography, MRI, or CT/CAT scan. In some aspects, thrombus formation is determined in organ tissue.

In some embodiments, reduction or prevention of pulmonary vascular leakage (i.e., lung leakage and damage) is a measurement of the effectiveness of the treatment. In some embodiments, bronchoalveolar lavage (BAL) measurements or parameters (total protein and leukocyte content) are measured as markers of pulmonary vascular leakage (to determine the extent of lung damage and effectiveness of treatment (e.g., treatment with ADAMTS13)). Pulmonary leakage can result in an increase in protein and/or leukocyte content in the BAL. BAL fluids are collected and cellular contents are recovered by centrifugation and counted by microcytometry as previously reported (Kalish et al., Haematologica 100:870-80, 2015, incorporated herein by reference in its entirety and for all purposes). In some embodiments, reduction or prevention of an increase in peripheral neutrophils is a measurement of the effectiveness of the treatment. The percentage of neutrophils is determined on cytospin centrifugation and the supernatant fluids are used for determination of total protein content (Kalish et al., supra).

In some embodiments, improvement of lung function is measured as an indicatory of treatment effectiveness. Lung function can be measured by, but not limited to, a peak flow test, a spirometry and reversibility test, a lung volume test, a gas transfer test, a respiratory muscle test, exhaled carbon monoside test, or an exhaled nitric oxide test.

In some embodiments, hematology parameters are measured to determine effectiveness of treatment (e.g., treatment with ADAMTS13). The following hematology parameters are determined: lactate dehydrogenase (LDH) as a general marker of cell damage; hematocrit (Hct) and mean corpuscular volume (MCV), as a measure of erythrocyte viability; hemoglobin (Hb), mean corpuscular hemoglobin (MCH) and cell hemoglobin concentration (CHCM), as indicators of oxygen binding capacity; heterogeneity of red cell distribution (HDW), as an indicator of the presence of dense red cells; reticulocyte count, as an indicator of anemia status; and neutrophil count, as an indicator of systemic inflammatory status.

In certain embodiments, administration of ADAMTS13 ameliorates the reduction of the levels of at least one of Hct, Hb, MCV and MCH in the blood as compared to control. In certain embodiments, administration of ADAMTS13 ameliorates the reduction of the levels of at least two of Hct, Hb, MCV and MCH in the blood as compared to control. In certain embodiments, administration of ADAMTS13 ameliorates the reduction of the levels of at least three of Hct, Hb, MCV and MCH in the blood as compared to control. In certain embodiments, administration of ADAMTS13 ameliorates the reduction of the levels of Hct, Hb, MCV and MCH in the blood as compared to control. In certain embodiments, administration of ADAMTS13 ameliorates the increase of at least one of CHCM, HDW, LDH, and neutrophil number as compared to control. In certain embodiments, administration of ADAMTS13 ameliorates the increase of at least two of CHCM, HDW, LDH, and neutrophil number as compared to control. In certain embodiments, administration of ADAMTS13 ameliorates the increase of at least three of CHCM, HDW, LDH, and neutrophil number as compared to control. In certain embodiments, administration of ADAMTS13 ameliorates the increase of CHCM, HDW, LDH, and neutrophil number as compared to control. In certain embodiments, ADAMTS13 ameliorates the reduction of Hct, Hb, MCV, and MCH levels and ameliorates the increase in CHCM, HDW, LDH, and neutrophil levels as compared to control.

In certain embodiments, administration of ADAMTS13 results in an increase in the levels of at least one of Hct, Hb, MCV and MCH in the blood as compared to control. In certain embodiments, administration of ADAMTS13 results in an increase in the levels of at least two of Hct, Hb, MCV and MCH in the blood as compared to control. In certain embodiments, administration of ADAMTS13 results in an increase in the levels of at least three of Hct, Hb, MCV and MCH in the blood as compared to control. In certain embodiments, administration of ADAMTS13 results in an increase in the levels of Hct, Hb, MCV and MCH in the blood as compared to control. In certain embodiments, administration of ADAMTS13 results in a decrease in at least one of CHCM, HDW, LDH, and neutrophil number as compared to control. In certain embodiments, administration of ADAMTS13 results in a decrease in at least two of CHCM, HDW, LDH, and neutrophil number as compared to control. In certain embodiments, administration of ADAMTS13 results in a decrease in at least three of CHCM, HDW, LDH, and neutrophil number as compared to control. In certain embodiments, administration of ADAMTS13 results in a decrease in CHCM, HDW, LDH, and neutrophil number as compared to control. In certain embodiments, ADAMTS13 results in an increase of Hct, Hb, MCV, and MCH levels and a reduction in CHCM, HDW, LDH, and neutrophil levels as compared to control.

In some embodiments, methods of measuring the levels of VWF and of ultra-large VWF multimers are used. In SCD patients, increased levels of VWF and of ultra-large VWF multimers have been observed and are associated with acute vaso-occlusive events. The increased levels of circulating VWF multimers are dependent on the activity of ADAMTS13 that cleaves the hyperadhesive ultra-large VWF under conditions of high fluid shear stress, playing an important role in maintaining a proper balance of hemostatic activity and thrombotic risk. More specifically, ADAMTS13 cleaves VWF between amino acid residues Tyr₁₆₀₅ and Met¹⁶⁰⁶, which corresponds to amino acid residues 842-843 after cleavage of the preprosequence. It is this ADAMTS13-mediated cleavage that is largely responsible for VWF multimer size, which correlates with primary hemostatic activity. Methods of measuring VWF and ultra-large VWF multimers, including various types of immunoblot analyses with specific antibodies against VWF, are carried out to measure the expression or level of VWF. Additionally, other known methods of measuring VWF are included in various aspects of the disclosure.

In certain embodiments, administration of ADAMTS13 results in a reduction in the levels of at least one of ultra-large VWF multimers, VWF activity and VWF activity/antigen ratio. VWF activity/antigen ratio is the ratio between VWF activity and VWF antigen in the plasma. VWF activity may be measured by various methods known in the art such as, but not limited to, VWF ristocetin cofactor activity assay and enzyme-immuno assay for measuring the collagen binding activity of VWF. VWF antigen levels may be measured using immunosorbent assays including commercially available ELISA tests (e.g., Asserachrom® VWF:Ag). In certain embodiments, administration of ADAMTS13 does not alter the level of VWF antigen in the plasma.

In certain embodiments, administration of ADAMTS13 results in an increase in ADAMTS13-mediated VWF cleavage. ADAMTS13-mediated VWF cleavage SCD patients can be inhibited due to an increased level of extracellular hemoglobin (ECHb) in the plasma. Extracellular hemoglobin in SCD patients may be present at a concentration of 20-330 μg/mL in the plasma, and >400 μg/mL during a VOC. In some embodiments, administration of ADAMTS13 results in an increase of ADAMTS13-mediated VWF cleavage by at least about 20% in a SCD patient. In some embodiments, administration of ADAMTS13 results in an increase of ADAMTS13-mediated VWF cleavage by at least about 20%, at least about 30%, at least about 40%, at least about 50%, at least about 60%, at least about 70%, at least about 80%, at least about 90%, or about 100% in a SCD patient. In some embodiments, administration of ADAMTS13 results in an increase of ADAMTS13-mediated VWF cleavage by about 20% to 70% in a SCD patient. In some embodiments, administration of ADAMTS13 results in an increase of ADAMTS13-mediated VWF cleavage by about 80% to 100% in a SCD patient.

In certain embodiments, administration of ADAMTS13 results in a reduction in the level of free hemoglobin in the plasma. Free hemoglobin may be measured using commercially available ELISA assays.

In some aspects, effectiveness is measured by decreased organ damage as compared to control or baseline measurements. In some embodiments, organ damage is measured by radiological imaging such as, but not limited to, CT/CAT scanning, ultrasound, X-ray, MRI, and nuclear medicine. In some embodiments, organ damage is measured by a change in various biomarkers including, but not limited to, blood urea nitrogen (BUN), creatinine, BUN/creatinine ratio, troponin, neuron-specific enolase (NSE). In some embodiments, tissue changes are measured by histopathological examination.

One of ordinary skill in the art is able to select an appropriate measure of any biomarker disclosed herein associated with the organ (defined above) and/or body fluid to be measured. Body fluids include, but are not limited to, blood (including blood plasma and blood serum), lymph, cerebrospinal fluid, lactation products (e.g., milk), amniotic fluids, urine, saliva, perspiration, tears, menses, feces, and including fractions thereof.

In some aspects, effectiveness is measured by assessing the Quality-of-Life of the subject (e.g., using the Adult Sickle Cell Quality-of-Life Measurement Information System (ASCQ-Me) as reported by Treadwell et al., Clin. J. Pain 30(10):902-915 (2016)). The ASCQ-Me centers around seven topics: emotional impact (five question survey related to emotional distress (e.g., hopelessness, loneliness, depression, and worry); pain episode frequency and severity (number of episodes, time since last episode; severity of pain in last attack on a scale from 1-10); how long did the attack last, how much did the attack impact your life); pain impact (asking about the frequency and severity and how it impacted activities); sickle cell disease medical history checklist; sleep impact (how easy to fall asleep, how often cannot fall asleep); social functioning impact (reliance on others, how health impacted activities); and stiffness impact (stiff joints causing sleeplessness, movement during the day, movement upon wakefulness).

In various aspects, effectiveness of prophylaxis and/or treatment is determined by measuring pain severity (e.g., as measured by a pain rating scale), pain relief, perceived need for medication, treatment satisfaction, the frequency of VOC occurrence, the duration of VOC episode, the length and/or duration of hospitalization, costs associated with a hospital stay, and/or the duration of the requirement for pain medication (e.g., i.v. opiates).

In certain aspects, pain severity is measured using the McGill/Melzack Pain Questionnaire (Melzack et al., Pain 1975 September; 1(3):277-99), in which the subject selects one or more words that best describe their pain. In certain aspects, pain severity is measured using the Visual Analog Scale (VAS). The VAS is a 10 cm, non-hatched line anchored with one end as “no pain” and the other end as “worst pain possible.” Patients are instructed to mark on the line their level of pain between the two anchors. VAS scores are calculated by measuring the distance, in centimeters, between the “no pain” anchor and the patient's mark indicating their level of pain resulting in a pain severity score ranging from 0 mm to 10 cm. In certain aspects, pain severity is measured using the Numeric Rating Scale (NRS). NRS is an 11-point scale anchored with “no pain” and “worst pain possible.” Patients are instructed to report their current level of pain on a scale from 0 to 10 where 0 means no pain and 10 means the worst pain possible.

In certain aspects, pain relief can be measured as a global assessment of how a patient's pain may have changed since the last assessment (i.e., current assessment minus previous assessment) as used to anchor the changes noted on the NRS and VAS scales. Patients reported pain relief in response to the question: “Compared to the last time you marked your pain, tell us how much your pain has changed.” Patients could respond that their pain was “a lot worse,” “a little worse,” “the same,” “a little better,” or “a lot better.”

In certain aspects, the need for medication can be patient or healthcare worker reported.

In certain aspects, treatment satisfaction can be a patient-reported. Reporting can be on a scale from “not at all,” “somewhat satisfied (happy),” “very satisfied (happy),” or “do not know.”

In certain aspects, effectiveness of prophylaxis and/or treatment for VOC in the mouse model is determined using a read-out through behavior observations. For example, one or more behavioral symptoms may be screened. In some embodiments, one or more behavioral symptoms are selected from piloerection, apathy, eyes appearance, skin color, spontaneous mobility, stimulated mobility, and breathing frequency. In some embodiments, one or more behavioral symptoms are selected from piloerection, apathy, eyes appearance, stimulated mobility, and breathing frequency. Additional behavioral symptoms may include those described in Mittal et al., Blood Cells Mol Dis. 57:58-66, 2016, which is herein incorporated by reference in its entirety. A behavioral score may be generated based on the severity of the behavioral symptoms. As a non-limiting example, the behavioral score may be generated according to the grading scale described in the SHIRPA guidelines (Rogers et al., Mamm Genome. 8(10):711-3, 1997, which is herein incorporated by reference in its entirety). In an exemplary embodiment, behavioral symptoms are scored such that higher numbers are assigned to more severe symptoms. The behavioral score may be compared to a control score to assess the effectiveness of prophylaxis and/or treatment. In some embodiments, the control score is generated from a control subject that does not receive the prophylaxis and/or treatment. The prophylaxis and/or treatment can be determined as effective if the behavioral score indicates less severity compared to a control score; or the prophylaxis and/or treatment can be determined as not effective if the behavioral score indicates more or the same severity compared to the control score.

In certain aspects, the recovery of a subject from a vaso-occlusive crisis (VOC) may be determined using a read-out through behavior observations. For example, one or more behavioral symptoms selected from piloerection, apathy, eyes appearance, skin color, spontaneous mobility, stimulated mobility, and breathing frequency may be collected from the subject after the VOC. A score may be generated based on the severity of the one or more behavioral symptoms collected from the subject. The score may be compared to a control score. The control score may be generated from a predetermined standard, or a healthy age- and gender-matched subject, or an average value for several such subjects. The control score may be generated from the subject before the VOC or from a control subject that does not have a VOC. The subject may be determined as have recovered from VOC if the score from the subject indicates less or the same severity compared to the control score; or the subject may be determined as not have recovered if the score from subject indicates more severity compared to the control score.

### ADAMTS13

In some aspects, the disclosure includes ADAMTS13 (also known as “A13”) and compositions comprising ADAMTS13 in the treatment and prevention of SCD. In particular aspects, the disclosure includes ADAMTS13 and compositions comprising ADAMTS13 in the treatment and prevention of VOC in SCD. The ADAMTS13 protease is about a 180 kDa to 200 kDa glycosylated protein produced predominantly by the liver. ADAMTS13 is a plasma metalloprotease which cleaves VWF multimers and down regulates their activity in platelet aggregation. To date, ADAMTS13 has been associated with clotting disorders, such as inherited thrombotic thrombocytopenic purpura (TTP), acquired TTP, cerebral infarction, myocardial infarction, ischemic/reperfusion injury, deep vein thrombosis, and disseminated intravascular coagulation (DIC), such as sepsis-related DIC.

All forms of ADAMTS13 known in the art are contemplated for use in the methods and uses of the disclosure. Mature ADAMTS13 has a calculated molecular mass of about 145 kDa whereas purified plasma-derived ADAMTS13 has an apparent molecular mass of about 180 kDa to 200 kDa, probably due to post-translational modifications consisting with present consensus sequences for 10 potential N-glycosylation sites, and several O-glycosylation sites and one C-mannosylation site in the TSP1 repeats.

As used herein, “ADAMTS13” refers to a metalloprotease of the ADAMTS (a disintegrin and metalloproteinase with thrombospondin type 1 motifs) family that cleaves VWF in the A2 domain between residues Tyr¹⁶⁰⁵ and Met¹⁶⁰⁶. In the context of the disclosure, “ADAMTS13”, “A13”, or an “ADAMTS13 protein” embraces any ADAMTS13 protein, for example, ADAMTS13 from a mammal such as a primate, human (NP620594), monkey, rabbit, pig, bovine (XP610784), rodent, mouse (NP001001322), rat (XP342396), hamster, gerbil, canine, feline, frog (NP001083331), chicken (XP415435), and biologically active derivatives thereof. As used herein, “ADAMTS13”, “A13”, or “ADAMTS13 protein” refers to recombinant, natural, or plasma-derived ADAMTS13 protein. Mutant and variant ADAMTS13 proteins having activity are also embraced, as are functional fragments and fusion proteins of the ADAMTS13 proteins. In some aspects, an ADAMTS13 protein further comprises a tag that facilitates purification, detection, or both. The ADAMTS13 protein of the disclosure, in some aspects, is further modified with an additional therapeutic moiety or a moiety suitable imaging in vitro or in vivo.

ADAMTS13 protein includes any protein or polypeptide with ADAMTS13 activity, particularly the ability to cleave the peptide bond between residues Tyr-842 and Met-843 of VWF. Human ADAMTS13 proteins include, without limitation, polypeptides comprising the amino acid sequence of GenBank accession number NP 620594 (NM139025.3) or a processed polypeptide thereof, for example a polypeptide in which the signal peptide (amino acids 1 to 29) and/or propeptide (amino acids 30-74) have been removed. In certain aspects, an ADAMTS13 protein refers to a polypeptide comprising an amino acid sequence that is highly similar to that of NP 620596 (ADAMTS13 isoform 2, preproprotein) or amino acids 75 to 1371 of P_620594 (ADAMTS13 isoform 2, mature polypeptide). In yet another embodiment, ADAMTS13 proteins include polypeptides comprising an amino acid sequence highly similar to that of NP 620595 (ADAMTS13 isoform 3, preproprotein) or amino acids 75 to 1340 of NP_620595 (ADAMTS13 isoform 1, mature polypeptide). In certain aspects, an ADAMTS13 protein includes natural variants with VWF cleaving activity and artificial constructs with VWF cleaving activity. In certain aspects, ADAMTS13 encompasses any natural variants, alternative sequences, isoforms or mutant proteins that retain some basal activity. Many natural variants of human ADAMTS13 are known in the art, and are embraced by the formulations of the disclosure, some of which include mutations selected from R7W, V88M, H96D, R102C, R193W, T196I, H234Q, A250V, R268P, W390C, R398H, Q448E, Q456H, P457L, P475S, C508Y, R528G, P618A, R625H, 1673F, R692C, A732V, E740K, A900V, S903L, C908Y, C951G, G982R, C1024G, A1033T, R1095W, R1095W, R1123C, C1213Y, T1226I, G1239V, and R1336W. Additionally, ADAMTS13 proteins include natural and recombinant proteins that have been mutated, for example, by one or more conservative mutations at a non-essential amino acid. Preferably, amino acids essential to the enzymatic activity of ADAMTS13 will not be mutated. These include, for example, residues known or presumed to be essential for metal binding such as residues 83, 173, 224, 228, 234, 281, and 284, and residues found in the active site of the enzyme, e.g., residue 225. Similarly, in the context of the disclosure, ADAMTS13 proteins include alternate isoforms, for example, isoforms lacking amino acids 275 to 305 and/or 1135 to 1190 of the full-length human protein.

In certain embodiments, the present disclosure includes variants of ADAMTS13. In certain embodiments, the ADAMTS13 variant comprises at least one single amino acid substitution as compared to the wildtype amino acid (e.g., SEQ ID NO: 1). In certain embodiments, the single amino acid substitution is within the catalytic domain of ADAMTS13 (e.g., amino acids 80 to 286 of SEQ ID NO: 1). In certain embodiments, the single amino acid substitution is at least one of I⁷⁹M, V⁸⁸M, H⁹⁶D, Q⁹⁷R, R¹⁰²C, S¹¹⁹F, I¹⁷⁸T, R¹⁹³W, T¹⁹⁶I, S²⁰³P, L²³²Q, H²³⁴Q, D²³⁵H, A²⁵⁰V, S²⁶³C, and/or R²⁶⁸P as denoted in SEQ ID NO: 1, or the equivalent amino acid in an ADAMTS13. In certain embodiments, the single amino acid substitution is not I⁷⁹M, V⁸⁸M, H⁹⁶D, R¹⁰²C, R¹⁹³W, T¹⁹⁶I, S²⁰³P, L²³²Q, H²³⁴Q, D²³⁵H, A²⁵⁰V, S²⁶³C, and/or R²⁶⁸P as denoted in SEQ ID NO: 1, or the equivalent amino acid in an ADAMTS13. In certain embodiments, the ADAMTS13 variant comprises a single amino acid substitution at Q⁹⁷ as denoted in SEQ ID NO: 1, or the equivalent amino acid in an ADAMTS13. In certain embodiments, the amino acid change is from a Q to a D, E, K, H, L, N, P, or R. In certain embodiments, the amino acid change is from a Q to an R. In certain embodiments, the ADAMTS13 variant is ADAMTS13 Q⁹⁷R (SEQ ID NO: 2).

In some aspects, ADAMTS13 proteins are further modified, for example, by post-translational modifications (e.g., glycosylation at one or more amino acids selected from human residues 142, 146, 552, 579, 614, 667, 707, 828, 1235, 1354, or any other natural or engineered modification site) or by ex vivo chemical or enzymatic modification, including without limitation, glycosylation, modification by water-soluble polymer (e.g., PEGylation, sialylation, HESylation, etc.), tagging, and the like.

In some aspects, the ADAMTS13 protein is human ADAMTS13 or a biologically active derivative or fragment thereof as described in U.S. Patent Application Publication No. 2011/0229455 and/or in U.S. Patent Application Publication No. 2014/0271611, each of which are incorporated herein by reference in their entirety and for all purposes.

In certain aspects, the recombinant ADAMTS13 can be BAX930/SHP655/TAK755. BAX930/SHP655/TAK755 is a fully glycosylated recombinant human ADAMTS13 protein (see e.g., WO2002042441, which is incorporated herein by reference in its entirety). In certain aspects, the ADAMTS13 protein includes any protein or polypeptide with ADAMTS13 activity, particularly the ability to cleave the peptide bond between residues Tyr-842 and Met-843 of VWF with at least 70%, at least 75%, at least 80%, at least 85%, at least 90%, at least 91%, at least 92%, at least 93%, at least 94%, at least 95%, at least 96%, at least 97%, at least 98%, or at least 99% sequence homology to BAX930/SHP655/TAK755.

Proteolytically active recombinant ADAMTS13 may be prepared by expression in mammalian cell cultures, as described in Plaimauer et al., (2002, Blood. 15; 100(10):3626-32) and US 2005/0266528, the disclosures of which are herein incorporated by reference in their entireties for all purposes. Methods for the expression of recombinant ADAMTS13 in cell culture are disclosed in Plaimauer B, Scheiflinger F. (Semin Hematol. 2004 January; 41(1):24-33 and US 2011/0086413, the disclosures of which are herein incorporated by reference in their entireties for all purposes). See also, WO2012/006594, incorporated by reference in their entireties for all purposes, for methods of producing recombinant ADAMTS13 in cell culture.

Methods for purifying ADAMTS13 protein from a sample are described in U.S. Pat. No. 8,945,895, which is incorporated herein by reference for all purposes. Such methods include, in some aspects, enriching for ADAMTS13 protein by chromatographically contacting the sample with hydroxyapatite under conditions that allow ADAMTS13 protein to appear in the eluate or supernatant from the hydroxyapatite. The methods may further comprise tandem chromatography with a mixed mode cation exchange/hydrophobic interaction resin that binds ADAMTS13 protein. Additional optional steps involve ultrafiltration/diafiltration, anion exchange chromatography, cation exchange chromatography, and viral inactivation. In some aspects, such methods include inactivating virus contaminants in protein samples, where the protein is immobilized on a support. Also provided herein, in some aspects, are compositions of ADAMTS13 prepared according to the methods described in U.S. Pat. No. 8,945,895.

### ADAMTS13 Compositions and Administration

In aspects of the disclosure, ADAMTS13 is administered to a subject in need thereof. To administer ADAMTS13 described herein to a subject, ADAMTS13 is, in some aspects, formulated in a composition comprising one or more pharmaceutically acceptable carriers.

The term “pharmaceutically acceptable,” as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans. “Pharmaceutically acceptable carriers” include any and all clinically useful solvents, dispersion media, coatings, antibacterial and antifungal agents, isotonic and absorption delaying agents and the like. In some aspects, the composition forms solvates with water or common organic solvents. Such solvates are included as well.

In some aspects, the disclosure provides stabilized formulations of plasma derived ADAMTS13 and recombinant ADAMTS13 (rADAMTS13) proteins as described in U.S. Pat. No. 8,623,352 and/or in U.S. Patent Application Publication No. 2014/0271611, both of which are incorporated herein by reference for all purposes. In some embodiments, the formulations provided herein retain significant ADAMTS13 activity when stored for extended periods of time. In some embodiments, the formulations of the disclosure reduce or retard dimerization, oligomerization, and/or aggregation of an ADAMTS13 protein.

In some aspects, the disclosure provides formulations of ADAMTS13 comprising a therapeutically effective amount or dose of an ADAMTS13 protein, a sub-physiological to physiological concentration of a pharmaceutically acceptable salt, a stabilizing concentration of one or more sugars and/or sugar alcohols, a non-ionic surfactant, a buffering agent providing a neutral pH to the formulation, and optionally a calcium and/or zinc salt. Generally, the stabilized ADAMTS13 formulations provided herein are suitable for pharmaceutical administration. In some aspects, the ADAMTS13 protein is human ADAMTS13 or a biologically active derivative or fragment thereof as described in U.S. Patent Application Publication No. 2011/0229455 and/or in U.S. Patent Application Publication No. 2014/0271611, each of which are incorporated herein by reference in their entirety and for all purposes.

In some aspects, the ADAMTS13 formulations are liquid or lyophilized formulations. In other embodiments, a lyophilized formulation is lyophilized from a liquid formulation as described in U.S. Patent Application Publication No. 2011/0229455 and/or in U.S. Patent Application Publication No. 2014/0271611, each of which are incorporated herein by reference in their entirety and for all purposes. In certain embodiments of the formulations provided herein, the ADAMTS13 protein is a human ADAMTS13 or recombinant human ADAMTS13, or a biologically active derivative or fragment thereof as described in U.S. Patent Application Publication No. 2011/0229455 and/or in U.S. Patent Application Publication No. 2014/0271611, each of which are incorporated herein by reference in their entirety and for all purposes.

The composition of the disclosure is, in various aspects, administered orally, topically, transdermally, parenterally, by inhalation spray, vaginally, rectally, or by intracranial injection. The term parenteral as used herein includes subcutaneous injections, intravenous, intramuscular, intracisternal injection, or infusion techniques. In some embodiments, administration is subcutaneous. Administration by intravenous, intradermal, intramuscular, intramammary, intraperitoneal, intrathecal, retrobulbar, intrapulmonary injection and or surgical implantation at a particular site is contemplated as well. In some embodiments, administration is intravenous. Generally, compositions are essentially free of pyrogens, as well as other impurities that could be harmful to the recipient.

Formulation of the composition or pharmaceutical composition will vary according to the route of administration selected (e.g., solution or emulsion). An appropriate composition comprising the composition to be administered is prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles, in some aspects, include sodium chloride solution, Ringer's dextrose, dextrose and sodium chloride, lactated Ringer's or fixed oils. Intravenous vehicles, in certain aspects, include various additives, preservatives, or fluid, nutrient or electrolyte replenishers.

Compositions or pharmaceutical compositions useful in the compounds and methods of the disclosure containing ADAMTS13 as an active ingredient contain, in various aspects, pharmaceutically acceptable carriers or additives depending on the route of administration. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form.

A variety of aqueous carriers, e.g., water, buffered water, 0.4% saline, 0.3% glycine, or aqueous suspensions contain, in various aspects, the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents, in some instances, are a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions, in some aspects, contain one or more preservatives, for example ethyl, or n-propyl, p-hydroxybenzoate.

In some aspects, ADAMTS13 or ADAMTS13 compositions are lyophilized for storage and reconstituted in a suitable carrier prior to use. Any suitable lyophilization and reconstitution techniques known in the art are employed. It is appreciated by those skilled in the art that lyophilization and reconstitution leads to varying degrees of protein activity loss and that use levels are often adjusted to compensate.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above.

In some embodiments, the ADAMTS13 formulations provided herein may further comprise one or more pharmaceutically acceptable excipients, carriers, and/or diluents as described in U.S. Patent Application Publication No. 2011/0229455 and/or in U.S. Patent Application Publication No. 2014/0271611, each of which are incorporated herein by reference in their entirety and for all purposes.

In some embodiments, the ADAMTS13 formulations provided herein will have a tonicity in a range described in as described in U.S. Patent Application Publication No. 2011/0229455 and/or in U.S. Patent Application Publication No. 2014/0271611, each of which are incorporated herein by reference in their entirety and for all purposes.

In some aspects, the disclosure provides formulations of ADAMTS13 comprising the exemplary formulations described in Section III (“ADAMTS13 Compositions and Formulations”) of U.S. Patent Application Publication No. 2011/0229455. The methods of ADAMTS13 production and compositions thereof as described in U.S. Patent Application Publication No. 2011/0229455 and/or in U.S. Patent Application Publication No. 2014/0271611 are incorporated herein by reference in their entirety for all purposes. Additionally, actual methods for preparing parenterally administrable formulations and compositions are known or are apparent to those skilled in the art and are described in more detail in, for example, Remington's Pharmaceutical Science, 15th ed., Mack Publishing Company, Easton, Pa. (1980).

In various aspects, the pharmaceutical compositions are in the form of a sterile injectable aqueous, oleaginous suspension, dispersions or sterile powders for the extemporaneous preparation of sterile injectable solutions or dispersions. The suspension, in some aspects, is formulated according to the known art using those suitable dispersing or wetting agents and suspending agents which have been mentioned above. The sterile injectable preparation, in certain aspects, is a sterile injectable solution or suspension in a non-toxic parenterally-acceptable diluent or solvent, for example as a solution in 1,3-butane diol. In some embodiments, the carrier is a solvent or dispersion medium containing, for example, water, ethanol, polyol (for example, glycerol, propylene glycol, and liquid polyethylene glycol, and the like), suitable mixtures thereof, vegetable oils, Ringer's solution and isotonic sodium chloride solution. In addition, sterile, fixed oils are conventionally employed as a solvent or suspending medium. For this purpose any bland fixed oil is employed, in various aspects, including synthetic mono- or diglycerides. In addition, fatty acids such as oleic acid find use in the preparation of injectables.

In all cases the form must be sterile and must be fluid to the extent that easy syringability exists. The proper fluidity is maintained, for example, by the use of a coating, such as lecithin, by the maintenance of the required particle size in the case of dispersion and by the use of surfactants. It must be stable under the conditions of manufacture and storage and must be preserved against the contaminating action of microorganisms, such as bacteria and fungi. The prevention of the action of microorganisms is brought about by various antibacterial and antifungal agents, for example, parabens, chlorobutanol, phenol, sorbic acid, thimerosal, and the like. In many cases, it will be desirable to include isotonic agents, for example, sugars or sodium chloride. In certain aspects, prolonged absorption of the injectable compositions is brought about by the use in the compositions of agents delaying absorption, for example, aluminum monostearate and gelatin.

Compositions useful for administration, in certain aspects, are formulated with uptake or absorption enhancers to increase their efficacy. Such enhancers include, for example, salicylate, glycocholate/linoleate, glycholate, aprotinin, bacitracin, SDS, caprate and the like. See, e.g., Fix (J. Pharm. Sci., 85:1282-1285, 1996) and Oliyai et al. (Ann. Rev. Pharmacol. Toxicol., 32:521-544, 1993), each of which are incorporated herein by reference in their entirety and for all purposes.

In addition, the properties of hydrophilicity and hydrophobicity of the compositions used in the compositions and methods of the disclosure are well balanced, thereby enhancing their utility for both in vitro and especially in vivo uses, while other compositions lacking such balance are of substantially less utility. Specifically, compositions in the disclosure have an appropriate degree of solubility in aqueous media which permits absorption and bioavailability in the body, while also having a degree of solubility in lipids which permits the compounds to traverse the cell membrane to a putative site of action.

In particular aspects, ADAMTS13 is provided in a pharmaceutically acceptable (i.e., sterile and non-toxic) liquid, semisolid, or solid diluent that serves as a pharmaceutical vehicle, excipient, or medium. Any diluent known in the art is used. Exemplary diluents include, but are not limited to, polyoxyethylene sorbitan monolaurate, magnesium stearate, methyl- and propylhydroxybenzoate, talc, alginates, starches, lactose, sucrose, dextrose, sorbitol, mannitol, gum acacia, calcium phosphate, mineral oil, cocoa butter, and oil of theobroma.

The composition is packaged in forms convenient for delivery. The composition is enclosed within a capsule, caplet, sachet, cachet, gelatin, paper, or other container. These delivery forms are preferred when compatible with delivery of the composition into the recipient organism and, particularly, when the composition is being delivered in unit dose form. The dosage units are packaged, e.g., in vials, tablets, capsules, suppositories, or cachets.

The disclosure includes methods for treating, ameliorating, and/or preventing VOC in SCD in a subject, including administering an effective amount of ADAMTS13 or an ADAMTS13 composition as described herein. The composition is introduced into the subject to be treated by any conventional method as described herein in detail above. In certain aspects, the composition is administered in a single dose or a plurality of doses over a period of time (as described in more detail below).

In some embodiments, the composition comprising ADAMTS13 is administered to the subject within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 60, 72, 84, 96, 108, or 120 hours after the onset of the VOC. In some embodiments, the composition comprising ADAMTS13 is administered to the subject within about 1-2 hours, about 1-5 hours, about 1-10 hours, about 1-12 hours, about 1-24 hours, about 1-36 hours, about 1-48 hour, about 1-60 hours, about 1-72 hours, about 1-84 hours, about 1-96 hours, about 1-108 hours, or about 1-120 hours after the onset of the VOC. In some embodiments, the composition comprising ADAMTS13 is administered to the subject within about 2-5 hours, about 5-10 hours, about 10-20 hours, about 20-40 hours, about 30-60 hours, about 40-80 hours, about 50-100 hours, or about 60-120 hours after the onset of the VOC. In some embodiments, the composition is administered within 1 week of the VOC. In some embodiments, the composition is administered daily after the VOC. In some embodiments, the composition is administered weekly after the VOC. In some embodiments, the composition is administered every day. In some embodiments, the composition is administered every other day. In some embodiments, the composition is administered every third day. In some embodiments, the composition is administered twice a week. In some embodiments, the composition is administered until the clinical manifestations (e.g., symptoms and/or biomarkers) resolve. In some embodiments, the composition is administered until a day after clinical manifestations resolve. In some embodiments, the composition is administered for at least two days after clinical manifestations resolve. In some embodiments, the composition is administered for at least three days after clinical manifestations resolve. In some embodiments, the composition is administered for at least a week after clinical manifestations resolve.

In some aspects, the composition comprising ADAMTS13 is administered to the subject suffering from sickle cell disease to prevent the onset of VOC. In such preventative treatment, ADAMTS13 is administered in a singular bolus injection or in multiple doses to maintain a circulating level of ADAMTS13 effective to prevent the onset of the VOC. In such aspects, the composition comprising ADAMTS13 is administered monthly, every two weeks, weekly, twice a week, every other day, or daily. In particular aspects, the injection is administered subcutaneously. In other aspects, the injection is administered intravenously.

In some embodiments, the composition comprising ADAMTS13 is administered to the subject before the onset of the VOC to prevent the VOC. In such aspects of the disclosure, the composition is administered in a therapeutically effective amount or dose sufficient to maintain an effective level of ADAMTS13 activity in the subject or in the blood of the subject.

### Dosing of ADAMTS13 Compositions/Methods of Treating

In various aspects, the effective dosage of ADAMTS13 or an ADAMTS13 composition to be administered varies depending on multiple factors which modify the action of drugs, e.g. the age, condition, body weight, sex, and diet of the subject, the severity of any infection, time of administration, mode of administration, and other clinical factors, including the severity of the VOC of the SCD.

In some aspects, formulations or compositions of the disclosure are administered by an initial bolus followed by booster delivery after a period of time has elapsed. In certain aspects, formulations of the disclosure are administered by an initial bolus followed by a continuous infusion to maintain therapeutic circulating levels of ADAMTS13. In particular aspects, ADAMTS13 or an ADAMTS13 composition of the disclosure is administered over extended periods of time. In some aspects, the ADAMTS13 or ADAMTS13 composition is delivered in a rapid treatment regimen to relieve acute symptoms of VOC. In some aspects, the ADAMTS13 or ADAMTS13 composition is delivered in a prolonged and varied treatment regimen to prevent the occurrence of VOC. As another example, the composition or formulation of the disclosure is administered as a one-time dose. Those of ordinary skill in the art readily optimize effective dosages and administration regimens as determined by good medical practice and the clinical condition of the individual subject. The frequency of dosing depends on the pharmacokinetic parameters of the agents, the route of administration, and the condition of the subject.

The pharmaceutical formulation is determined by one skilled in the art depending upon the route of administration and desired dosage. See for example, Remington's Pharmaceutical Sciences, 18th Ed. (1990, Mack Publishing Co., Easton, Pa. 18042) pages 1435-1712, the disclosure of which is hereby incorporated by reference for all purposes. Such formulations, in some instances, influence the physical state, stability, rate of in vivo release, and rate of in vivo clearance of the administered composition. Depending on the route of administration, a suitable dose is calculated, in particular aspects, according to body weight, body surface area or organ size. In some aspects, appropriate dosages are ascertained through use of established assays for determining blood level dosages in conjunction with appropriate dose-response data. In certain aspects, the antibody titer of an individual is measured to determine optimal dosage and administration regimens. The final dosage regimen will be determined by the attending doctor or physician, considering various factors which modify the action of the pharmaceutical compositions, e.g. the composition's specific activity, the responsiveness of the subject, the age, condition, body weight, sex and diet of the subject, the severity of any infection or malignant condition, time of administration and other clinical factors, including the severity of the pain or the VOC.

In certain aspects, the ADAMTS13 or ADAMTS13 composition comprises any dose of ADAMTS13 sufficient to evoke a response in the subject. In some embodiments, the dose of ADAMTS13 is sufficient to treat VOC. In some embodiments, the dose of ADAMTS13 is sufficient to prevent VOC. The effective amount of ADAMTS13 or ADAMTS13 composition to be employed therapeutically will depend, for example, upon the therapeutic context and objectives. One skilled in the art will appreciate that the appropriate dosage levels for treatment or prevention will thus vary depending, in part, upon the molecule delivered, the indication for which the ADAMTS13 or ADAMTS13 composition is being used, the route of administration, and the size (body weight, body surface or organ size) and condition (the age and general health) of the patient. Accordingly, the clinician, in some instances, titers the dosage and modifies the route of administration to obtain the optimal therapeutic effect.

Dosage, unless otherwise specifically recited, is provided in international units. As discussed herein below, the use of international units (IU) is the new standard for measuring ADAMTS13 activity. Up until recently, FRETS units (or FRETS-VWF73 test units) were the standard for measuring ADAMTS13 activity. 20 FRETS units (FRETS U) is equivalent to approximately 21.78 IU. In other words, 20 IU of ADAMTS13 is equivalent to about 18.22 FRETS U of ADAMTS13.

A typical dosage, in various aspects, ranges from about 10 international units per kilogram body weight up to about 10,000 international units per kilogram body weight. In some aspects, a dosage or therapeutically effective amount of ADAMTS13 is up to about 10,000 international units per kilogram body weight or more, depending on the factors mentioned above. In other aspects, the dosage may range from about 20 to about 6,000 international units per kilogram body weight. In some aspects, the dosage or therapeutically effective amount of ADAMTS13 is from about 40 to about 4,000 international units per kilogram body weight. In some aspects, the dosage or therapeutically effective amount is from about 100 to about 3,000 international units per kilogram body weight.

In particular aspects, the dosage or therapeutically effective amount is from about 10 to about 500 international units per kilogram body weight. In some aspects, the dosage or therapeutically effective amount is from about 50 to about 450 international units per kilogram body weight. In some aspects, the therapeutically effective amount is from about 40 to about 100 international units per kilogram body weight. In some aspects, the therapeutically effective amount is from about 40 to about 150 international units per kilogram body weight. In some aspects, the dosage or therapeutically effective amount is from about 100 to about 500 international units per kilogram body weight. In some aspects, the dosage or therapeutically effective amount is from about 100 to about 400 international units per kilogram body weight. In some aspects, the dosage or therapeutically effective amount is from about 100 to about 300 international units per kilogram body weight. In some aspects, the dosage or therapeutically effective amount is from about 300 to about 500 international units per kilogram body weight. In some aspects, the dosage or therapeutically effective amount is from about 200 to about 300 international units per kilogram body weight. In some aspects, the dosage or therapeutically effective amount is about 100, about 150, about 200, about 250, about 300, about 350, about 400, about 450, or about 500 international units per kilogram body weight.

In further aspects, the dosage or therapeutically effective amount is from about 50 to about 1,000 international units per kilogram body weight. In some aspects, the dosage or therapeutically effective amount is from about 100 to about 900 international units per kilogram body weight. In some aspects, the dosage or therapeutically effective amount is from about 200 to about 800 international units per kilogram body weight. In some aspects, the dosage or therapeutically effective amount is from about 300 to about 700 international units per kilogram body weight. In some aspects, the dosage or therapeutically effective amount is from about 400 to about 600 international units per kilogram body weight. In some aspects, the dosage or therapeutically effective amount is about 500 international units per kilogram body weight.

In some aspects, the dosage or therapeutically effective amount is about 10 international units per kilogram body weight, about 20 international units per kilogram body weight, about 30 international units per kilogram body weight, about 40 international units per kilogram body weight, about 50 international units per kilogram body weight, about 60 international units per kilogram body weight, about 70 international units per kilogram body weight, about 80 international units per kilogram body weight, about 90 international units per kilogram body weight, about 100 international units per kilogram body weight, about 120 international units per kilogram body weight, about 140 international units per kilogram body weight, about 150 international units per kilogram body weight, about 160 international units per kilogram body weight, about 180 international units per kilogram body weight, about 200 international units per kilogram body weight, about 220 international units per kilogram body weight, about 240 international units per kilogram body weight, about 250 international units per kilogram body weight, about 260 international units per kilogram body weight, about 280 international units per kilogram body weight, about 300 international units per kilogram body weight, about 350 international units per kilogram body weight, about 400 international units per kilogram body weight, about 450 international units per kilogram body weight, about 500 international units per kilogram body weight, about 550 international units per kilogram body weight, about 600 international units per kilogram body weight, about 650 international units per kilogram body weight, about 700 international units per kilogram body weight, about 750 international units per kilogram body weight, about 800 international units per kilogram body weight, about 850 international units per kilogram body weight, about 900 international units per kilogram body weight, about 950 international units per kilogram body weight, about 1,000 international units per kilogram body weight, about 1,100 international units per kilogram body weight, about 1,100 international units per kilogram body weight, about 1,200 international units per kilogram body weight, about 1,300 international units per kilogram body weight, about 1,400 international units per kilogram body weight, about 1,500 international units per kilogram body weight, about 1,600 international units per kilogram body weight, about 1,800 international units per kilogram body weight, about 2,000 international units per kilogram body weight, about 2,500 international units per kilogram body weight, about 3,000 international units per kilogram body weight, about 3,500 international units per kilogram body weight, about 4,000 international units per kilogram body weight, about 4,500 international units per kilogram body weight, about 5,000 international units per kilogram body weight, about 5,500 international units per kilogram body weight, about 6,000 international units per kilogram body weight, about 6,500 international units per kilogram body weight, about 7,000 international units per kilogram body weight, about 7,500 international units per kilogram body weight, about 8,000 international units per kilogram body weight, about 8,500 international units per kilogram body weight, about 9,000 international units per kilogram body weight, about 9,500 international units per kilogram body weight, and about 10,000 international units per kilogram body weight.

As used herein, “one unit of ADAMTS13 activity” or “one activity unit” is defined as the amount of activity in 1 mL of pooled normal human plasma, regardless of the assay being used. As provided above, however, the new standard for measuring or dosing ADAMTS13 is international units (IU). 20 FRETS test units or 20 FRETS units (FRETS U) is equivalent to approximately 21.78 IU. In other words, 20 IU of ADAMTS13 is equivalent to about 18.22 FRETS U of ADAMTS13. Thus, the change to the new standard results in an approximate shift of 8.9% in the conversion of FRETS U to IU.

In some aspects, fluorescence resonance energy transfer (FRET) assays are used to measure ADAMTS13 activity. FRET requires two interacting partners of which one is labeled with a donor fluorophore and the other is labeled with an acceptor fluorophore. FRET assays for ADAMTS13 involve a chemically modified fragment of the A2 domain of VWF which spans the ADAMTS13 cleavage site. This is readily cleaved by normal plasma but not by ADAMTS13 deficient plasma. This cleavage is blocked by EDTA and so samples for this assay must be collected into tubes that contain citrate as an anticoagulant and not EDTA. One unit of ADAMTS13 FRETS-VWF73 activity is the amount of activity needed to cleave the same amount of FRETS-VWF73 substrate (Kokame et al., Br J. Haematol. 2005 April; 129(1):93-100, incorporated herein by reference in its entirety) as is cleaved by one mL of pooled normal human plasma.

In some aspects, additional activity assays are used for measuring the activity of ADAMTS13. For example, direct ADAMTS13 activity assays can be performed to detect the cleavage of either full-length VWF molecules or VWF fragments using SDS agarose gel electrophoresis and indirect detection of ADAMTS13 activity can be detected with collagen binding assays. Direct assays, including the FRET assay, as described herein, involve the detection of cleavage of products either of a full-length VWF molecule or a VWF fragment that encompasses the ADAMTS13 cleavage site. With SDS agarose gel electrophoresis and Western Blotting, purified VWF is incubated with plasma for 24 hours. Cleavage of the VWF by ADAMTS13 takes place leading to a reduction in multimer sizes. This reduction is visualized by agarose gel electrophoresis followed by Western blotting with a peroxidase-conjugated anti-VWF antibody. The concentration of ADAMTS13 activity in the test sample can be established by reference to a series of diluted normal plasma samples. SDS-PAGE and Western Blotting can also be carried out, which involves the visualization of dimeric VWF fragments following SDS PAGE and Western Blotting. The assay is technically easier than SDS agarose gel electrophoresis and appears a very sensitive method for measuring ADAMTS13 activity levels.

In some aspects, indirect assays involve the detection of cleavage of products either of a full-length VWF molecule or a VWF fragment that encompasses the ADAMTS13 cleavage site in the A2 domain of VWF. Such assays include collagen binding assays, where normal plasma or purified VWF is incubated with the test plasma sample in the presence of BaC12 and 1.5M urea which denatures the VWF. VWF is cleaved by ADAMTS13 and residual VWF is measured by its binding to collagen Type III. The bound VWF is quantitated using an ELISA assay with a conjugated anti-VWF antibody. Another indirect assay is the ristocetin-induced aggregation assay. This is similar to the collagen-binding assay above but residual VWF is measured by ristocetin-induced platelet aggregation using a platelet aggregometer. Another indirect assay is a functional ELISA. In this assay, a recombinant VWF fragment is immobilized onto an ELISA plate using an antibody to a tag on the VWF. The VWF fragment encodes the A2 domain and the ADAMTS13 cleavage site at Tyr1605-Met1606 and is tagged with S-transferase [GST]-histidine [GST-VWF73-His]. Plasma is added to the immobilized GST-VWF73-His fragment and cleavage of the immobilized fragment occurs at the ADAMTS13 cleavage site. The residual, cleaved VWF fragment is measured by using a second monoclonal antibody that recognizes only the cleaved VWF fragment and NOT the interact fragment. ADAMTS13 activity is, therefore, inversely proportional to the residual substrate concentration.

ADAMTS13 activity may be assessed by ADAMTS13 functional assays (see e.g., Peyvandi et al., J Thromb Haemost; 8: 631-40, 2010). Exemplary functional assays may use full-length VWF under moderate denaturing conditions (e.g., in the presence of urea or guanidine hydrochloride) to unfold the VWF substrate and to make it susceptible for ADAMTS13 cleavage, or utilize short peptidyl substrates (such as the VWF73 substrate) (Kokame et al., Blood; 103(2): 607-12, 2004; Kokame et al., Br J Haematol; 129(1): 93-100, 2005; each of which are herein incorporated by reference in its entirety). Such small peptide substrates are derived from the A2 domain of VWF and contain the minimal VWF amino acid region required to be recognized and cleaved by ADAMTS13 as substrate (Kokame et al., Br J Haematol; 129(1): 93-100, 2005, which is incorporated herein by reference in its entirety).

In certain embodiments, a flow-based assay (see e.g., Han et al., Transfusion; 51(7): 1580-91, 2011, which is incorporated herein by reference in its entirety) is used to assess ADAMTS13 activity. The assay mimics the in vivo physiologic flow conditions necessary to achieve conformational changes of the full-length VWF substrate required for ADAMTS13 binding and ADAMTS13-mediated cleavage (Shim et al., Blood; 111(2): 651-7, 2008, which is incorporated herein by reference in its entirety).

In certain embodiments, ADAMTS13 is provided or administered in a therapeutically effective concentration between about 0.05 mg/mL and about 10 mg/mL in the final formulation. In other embodiments, ADAMTS13 is present at a concentration of between about 0.1 mg/mL and about 10 mg/mL. In yet other embodiments, ADAMTS13 is present at a concentration of between about 0.1 mg/mL and about 5 mg/mL. In another embodiment, ADAMTS13 is present at a concentration of between about 0.1 mg/mL and about 2 mg/mL. In yet other embodiments, ADAMTS13 may be present at about 0.01 mg/mL, or at about 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, or a higher concentration.

In some embodiments, the concentration of a relatively pure ADAMTS13 formulation may be determined by spectroscopy (i.e., total protein measured at A280) or other bulk determination (e.g., Bradford assay, silver stain, weight of a lyophilized powder, etc.). In other embodiments, the concentration of ADAMTS13 may be determined by an ADAMTS13 ELISA assay (e.g., mg/mL antigen).

In some aspects, the concentration of ADAMTS13 in a formulation of the disclosure is expressed as a level of enzymatic activity. For example, in some embodiments, an ADAMTS13 formulation contains between about 10 units of FRETS-VWF73 activity and about 10,000 units of FRETS-VWF73 activity or other suitable ADAMTS13 enzymatic unit (IU). In other embodiments, the formulation may contain between about 20 units of FRETS-VWF73 (U_{FV73}) activity and about 8,000 units of FRETS-VWF73 activity, or between about 30 U_{FV73} and about 6,000 U_{FV73}, or between about 40 U_{FV73} and about 4,000 U_{FV73}, or between about 50 U_{FV73} and about 3,000 U_{FV73}, or between about 75 U_{FV73} and about 2,500 U_{FV73}, or between about 100 U_{FV73} and about 2,000 U_{FV73}, or between about 200 U_{FV73} and about 1,500 U_{FV73}, or between about other ranges therein.

In some embodiments, ADAMTS13 is provided or administered at a dose of from about 10 U_{FV73}/kg body weight to 10,000 U_{FV73}/kg body weight. In one embodiment, ADAMTS13 is administered at a dose of from about 20 U_{FV73}/kg body weight to about 8,000 U_{FV73}/kg body weight. In one embodiment, ADAMTS13 is administered at a dose of from about 30 U_{FV73}/kg body weight to about 6,000 U_{FV73}/kg body weight. In one embodiment, ADAMTS13 is administered at a dose of from about 40 U_{FV73}/kg body weight to about 4,000 U_{FV73}/kg body weight. In one embodiment, ADAMTS13 is administered at a dose of from about 100 U_{FV73}/kg body weight to about 3,000 U_{FV73}/kg body weight. In one embodiment, ADAMTS13 is administered at a dose of from about 200 U_{FV73}/kg body weight to about 2,000 U_{F}v₇₃/kg body weight. In other embodiments, ADAMTS13 is administered at about 10 U_{FV73}/kg body weight, about 20, 30, 40, 50, 60, 70, 80, 90, 100, 150, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,500, 5,000, 5,500, 6,000, 6,500, 7,000, 7,500, 8,000, 8,500, 9,000, 9,500, or 10,000 U_{F}v₇₃/kg body weight, or at an intermediate dose or dose range thereof.

In some aspects, an ADAMTS13 formulation provided herein contains between about 20 and about 10,000 U_{FV73}. In some embodiments, a formulation contains about 10 units of FRETS-VWF73 activity, or about 20, 30, 40, 50, 60, 70, 80, 90, 100, 150, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more units of FRETS-VWF73 activity.

In some aspects, the concentration of ADAMTS13 may be expressed as an enzymatic activity per unit volume, for example, ADAMTS13 enzymatic units per mL (IU/mL). For example, in some embodiments, an ADAMTS13 formulation contains between about 10 IU/mL and about 10,000 IU/mL. In some other embodiments, the formulation contains between about 20 IU/mL and about 10,000 IU/mL, or between about 20 IU/mL and about 8,000 IU/mL, or between about 30 IU/mL and about 6,000 IU/mL, or between about 40 IU/mL and about 4,000 IU/mL, or between about 50 IU/mL and about 3,000 IU/mL, or between about 75 IU/mL and about 2,500 IU/mL, or between about 100 IU/mL and about 2,000 IU/mL, or between about 200 IU/mL and about 1,500 IU/mL, or between about other ranges therein. In some embodiments, an ADAMTS13 formulation provided herein contains between about 150 IU/mL and about 600 IU/mL. In another embodiment, an ADAMTS13 formulation provided herein contains between about 100 IU/mL and about 1,000 IU/mL. In some embodiments, an ADAMTS13 formulation provided herein contains between about 100 IU/mL and about 800 IU/mL. In some embodiments, an ADAMTS13 formulation provided herein contains between about 100 IU/mL and about 600 IU/mL. In some embodiments, an ADAMTS13 formulation provided herein contains between about 100 IU/mL and about 500 IU/mL. In some embodiments, an ADAMTS13 formulation provided herein contains between about 100 IU/mL and about 400 IU/mL. In some embodiments, an ADAMTS13 formulation provided herein contains between about 100 IU/mL and about 300 IU/mL. In some embodiments, an ADAMTS13 formulation provided herein contains between about 100 IU/mL and about 200 IU/mL. In some embodiments, an ADAMTS13 formulation provided herein contains between about 300 IU/mL and about 500 IU/mL. In some embodiments, an ADAMTS13 formulation provided herein contains about 100 IU/mL. In some embodiments, an ADAMTS13 formulation provided herein contains about 300 IU/mL. In various embodiments, a formulation contains about 10 IU/mL, or about 20, 30, 40, 50, 60, 70, 80, 90, 100, 150, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,100, 3,200, 3,300, 3,400, 3,500, 3,600, 3,700, 3,800, 3,900, 4,000, 4,100, 4,200, 4,300, 4,400, 4,500, 4,600, 4,700, 4,800, 4,900, 5,000, 5,100, 5,200, 5,300, 5,400, 5,500, 5,600, 5,700, 5,800, 5,900, 6,000, 6,100, 6,200, 6,300, 6,400, 6,500, 6,600, 6,700, 6,800, 6,900, 7,000, 7,100, 7,200, 7,300, 7,400, 7,500, 7,600, 7,700, 7,800, 7,900, 8,000, 8,100, 8,200, 8,300, 8,400, 8,500, 8,600, 8,700, 8,800, 8,900, 9,000, 9,100, 9,200, 9,300, 9,400, 9,500, 9,600, 9,700, 9,800, 9,900, 10,000 or more IU/mL.

In some embodiments, administering ADAMTS13 or a composition comprising ADAMTS13 results in a desired plasma ADAMTS13 concentration. The plasma ADAMTS13 concentration may be determined after a certain period of time (e.g., 5 minutes, 1 hour, 3 hours or 24 hours) post administration. In some embodiments, administering ADAMTS13 or a composition comprising ADAMTS13 results a plasma ADAMTS13 concentration of about 0.5 to about 100 U/mL in the subject. For example, in some embodiments, administering ADAMTS13 or a composition comprising ADAMTS13 results in a plasma ADAMTS13 concentration of about 1 to about 80 U/mL in the subject. In some embodiments, administering ADAMTS13 or a composition comprising ADAMTS13 results in a plasma ADAMTS13 concentration of about 5 to about 50 U/mL in the subject. In some embodiments, administering ADAMTS13 or a composition comprising ADAMTS13 results in a plasma ADAMTS13 concentration of about 12 to about 50 U/mL in the subject. In some embodiments, administering ADAMTS13 or a composition comprising ADAMTS13 results in a plasma ADAMTS13 concentration of about 5 to about 20 U/mL in the subject.

In some embodiments, administering ADAMTS13 or a composition comprising ADAMTS13 results in a plasma ADAMTS13 concentration of about 1 U/mL, about 2 U/mL, about 3 U/mL, about 4 U/mL, about 5 U/mL, about 6 U/mL, about 7 U/mL, about 8 U/mL, about 9 U/mL, about 10 U/mL, about 11 U/mL, about 12 U/mL, about 13 U/mL, about 14 U/mL, about 15 U/mL, about 16 U/mL, about 17 U/mL, about 18 U/mL, about 19 U/mL, about 20 U/mL, about 21 U/mL, about 22 U/mL, about 22 U/mL, about 23 U/mL, about 24 U/mL, about 25 U/mL, about 26 U/mL, about 27 U/mL, about 28 U/mL, about 29 U/mL, about 30 U/mL, about 32 U/mL, about 34 U/mL, about 36 U/mL, about 38 U/mL, about 40 U/mL, about 42 U/mL, about 44 U/mL, about 46 U/mL, about 48 U/mL, about 50 U/mL, about 52 U/mL, about 54 U/mL, about 56 U/mL, about 58 U/mL, about 60 U/mL, about 70 U/mL, about 80 U/mL, or more than 80 U/mL in the subject.

In some embodiments, the ADAMTS13 formulations provided herein may further comprise one or more pharmaceutically acceptable excipients, carriers, and/or diluents as described in U.S. Patent Application Publication No. 2011/0229455 and/or in U.S. Patent Application Publication No. 2014/0271611, each of which incorporated by reference in their entirety for all purposes. Furthermore, in one embodiment, the ADAMTS13 formulations provided herein will have a tonicity in a range described in as described in U.S. Patent Application Publication No. 2011/0229455 and/or in U.S. Patent Application Publication No. 2014/0271611, each of which incorporated by reference in their entirety for all purposes.

The frequency of dosing will depend upon the pharmacokinetic parameters of the ADAMTS13 molecule in the formulation used. Typically, a clinician will administer the composition until a dosage is reached that achieves the desired effect. The composition, in various aspects, is therefore administered as a single dose, or as two or more doses (which may or may not contain the same amount of the desired molecule) over time, or as a continuous infusion via an implantation device or catheter. In some aspects, the composition comprising ADAMTS13 is administered in a single bolus injection, monthly, every two weeks, weekly, twice a week, every other day, daily, every 12 hours, every eight hours, every six hours, every four hours, or every two hours. In the prophylactic or preventative treatment aspects of the disclosure, ADAMTS13 is administered in multiple doses to maintain a circulating level of ADAMTS13 effective to prevent the onset of the VOC. In such aspects, the composition comprising ADAMTS13 is administered monthly, every two weeks, weekly, twice a week, every other day, or daily. In particular aspects, the injection is administered subcutaneously (e.g., WO2014151968, incorporated herein by reference in its entirety for all purposes). In other aspects, the injection is administered intravenously. Further refinement of the appropriate dosage administered and the timing of administration is routinely made by those of ordinary skill in the art and is within the ambit of tasks routinely performed by them. Appropriate dosages are often ascertained through use of appropriate dose-response data which is routinely obtained.

### Kits Comprising ADAMTS13

As an additional aspect, the disclosure includes kits which comprise one or more pharmaceutical formulations for administration of ADAMTS13 or an ADAMTS13 composition to a subject packaged in a manner which facilitates their use for administration to the subject.

In a specific embodiment, the disclosure includes kits for producing a single dose administration unit. In another embodiment, the disclosure includes kits for providing multiple dose administration units. The kits, in various aspects, each contain both a first container having a dried protein and a second container having an aqueous formulation. Also included within the scope of this disclosure are kits containing single and multi-chambered pre-filled syringes (e.g., liquid syringes and lyosyringes).

In another embodiment, such a kit includes a pharmaceutical formulation described herein (e.g., a composition comprising a therapeutic protein, e.g., ADAMTS13), packaged in a container such as a sealed bottle or vessel, with a label affixed to the container or included in the package that describes use of the compound or composition in practicing the method. In one embodiment, the pharmaceutical formulation is packaged in the container such that the amount of headspace in the container (e.g., the amount of air between the liquid formulation and the top of the container) is very small. Preferably, the amount of headspace is negligible (i.e., almost none).

In some aspects, the pharmaceutical formulation or composition comprises a stabilizer. The term “stabilizer” refers to a substance or excipient which protects the composition from adverse conditions, such as those which occur during heating or freezing, and/or prolongs the stability or shelf-life of the composition or pharmaceutical composition in a stable state. Examples of stabilizers include, but are not limited to, sugars, such as sucrose, lactose and mannose; sugar alcohols, such as mannitol; amino acids, such as glycine or glutamic acid; and proteins, such as human serum albumin or gelatin.

In some aspects, the pharmaceutical formulation or composition comprises an antimicrobial preservative. The term “antimicrobial preservative” refers to any substance which is added to the composition that inhibits the growth of microorganisms that may be introduced upon repeated puncture of multidose vials, should such containers be used. Examples of antimicrobial preservatives include, but are not limited to, substances such as thimerosal, 2-phenoxyethanol, benzethonium chloride, and phenol.

In one aspect, the kit contains a first container having a therapeutic protein or protein composition and a second container having a physiologically acceptable reconstitution solution for the composition. In one aspect, the pharmaceutical formulation is packaged in a unit dosage form. The kit optionally further includes a device suitable for administering the pharmaceutical formulation according to a specific route of administration. In some aspects, the kit contains a label that describes use of the pharmaceutical formulations.

This entire document is intended to be related as a unified disclosure, and it should be understood that all combinations of features described herein are contemplated, even if the combination of features are not found together in the same sentence, or paragraph, or section of this document. The disclosure also includes, for instance, all embodiments of the disclosure narrower in scope in any way than the variations specifically mentioned above.

All publications, patents and patent applications cited in this specification are herein incorporated by reference as if each individual publication or patent application were specifically and individually indicated to be incorporated by reference in its entirety to the extent that it is not inconsistent with the disclosure.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Additional aspects and details of the disclosure will be apparent from the following examples, which are intended to be illustrative rather than limiting.

### Example 1

### Proteolytic Activity of Recombinant ADAMTS13 in the Presence of Hemoglobin

The objective of this study was to evaluate (i) the inhibitory effect of hemoglobin on ADAMTS13-mediated VWF multimer cleavage; (ii) if recombinant ADAMTS13 (rADAMTS13 [also known as SHP655 or BAX930 or TAK755]) in excess amounts can prevent the inhibitory effect or override it; and (iii) the human rADAMTS13 (SHP655) concentrations necessary to prevent or override this inhibitory effect. This study was aimed to show in vitro feasibility of rADAMTS13 supplementation in sickle cell disease (SCD) patients where elevated extracellular hemoglobin impairs VWF multimer cleavage.

ADAMTS13 activity is inhibited by the high plasma concentrations of free hemoglobin (Hb) commonly observed in SCD. It was shown that extracellular hemoglobin (ECHb) binds to the von Willebrand factor (VWF) A2 domain and significantly prohibits its cleavage by ADAMTS13. To mimic the described inhibitory effect of extracellular hemoglobin on ADAMTS13-mediated VWF multimer cleavage under non-denaturing assay flow conditions, a vortex-based methodology using full-length VWF as substrate was used. ADAMTS13-mediated VWF proteolytic cleavage products were analyzed in VWF-specific immunoblots after incubation of a reaction mixture consisting of full length recombinant VWF (rVWF), hemoglobin, lyophilized formalin-fixed platelets and recombinant ADAMTS13 (rADAMTS13) at constant vortexing. Additionally, it was investigated if rADAMTS13 in surplus amounts can override the blocking effect of hemoglobin and thus enable ultra-large VWF (ULVWF) multimer degradation.

### 1. Vortex-Based Assay

The vortex-based assay was established to determine ADAMTS13 activity under fluid shear stress using full-length VWF substrate (Han et al., Transfusion; 51(7): 1580-91, 2011; Shim et al., Blood; 111(2): 651-7, 2008; each of which is herein incorporated by reference in its entirety). In the assay, first described by Han et al. (Han et al., Transfusion; 51(7): 1580-91, 2011), rVWF is incubated together with formalin-fixed washed platelets and the ADAMTS13 test sample at constant vortexing. The generated VWF cleavage fragments are then separated by sodium dodecyl sulfate polyacrylamide gel electrophoresis (SDS-PAGE), detected by VWF-specific immunoblot analysis and quantified by densitometry.

The vortex-based assay was performed following a standard protocol. In brief, the reaction mixture (containing rVWF, platelets, hemoglobin and rADAMTS13 in vortex assay buffer with a total volume of 60 μL) was transferred into a 0.2 mL thin-wall reaction tube and incubated for 60 minutes at room temperature (RT) under constant vortexing at a rotation rate of 2500 rpm on the MixMate vortexer. Afterwards, all reaction mixtures were stopped by adding ethylenediaminetetraacetate (EDTA) to a final concentration of 10 mM.

VWF cleavage fragments (dimeric fragments of 176 kDa and 140 kDa) were separated on NuPage 3-8% Tris-acetate gels under non-reducing conditions and visualized by immunoblotting using a polyclonal rabbit anti-VWF antibody conjugated to HRP, and evaluated by densitometric analyses of the dimeric 176 kDa cleavage fragment.

All reaction mixtures with hemoglobin were compared to the reaction mixtures without addition of hemoglobin treated in the same way.

1.1 Preparation of Platelets

Formalin-fixed lyophilized platelets (Helena, Catalog #5371) were dissolved in 3 mL of platelet dissolving buffer (20 mM Tris, 100 mM NaCl buffer, pH 7.4), incubated for 10 minutes at RT and centrifuged for 5 minutes at 10000 rpm. The platelet pellet was resuspended in vortex assay buffer (50 mM HEPES, 150 mM NaCl, 0.1 μM ZnC12, 5 mM CaCl2, 0.3% BSA, pH 7.4) and the concentration of the platelets was determined using the Sysmex PocH 100i blood analysis system (Sysmex; Kobe, Japan). Platelets, dissolved in vortex assay buffer, were stored for up to 24 hours at 4° C. according to the manufacturer's specification. Platelets were used in the reaction mixture at a final concentration of 300×10³ cells/4.

1.2 Reconstitution of rVWF

One vial of rVWF (Lot: HN4AR00) lyophilizate was dissolved in 500 μL distilled deionized water resulting in a concentration of 91 IU/mL (VWF:Antigen activity). The rVWF was then pre-diluted in vortex assay buffer to a concentration of 18 IU/mL and further diluted 1:6 in the reaction mixture. The final rVWF concentration used in each reaction mixture was 3 IU/mL, which corresponds to roughly 30 μg/mL.

1.3 Preparation of Hemoglobin Solution

Hemoglobin powder (Sigma, Catalog #H7397; prepared from human erythrocytes) was dissolved in vortex assay buffer to concentrations of 100 mg/mL, 10 mg/mL and 1 mg/mL; and the intended volume was added to the reaction mixture to reach the final concentrations of 0.1 mg/mL, 0.5 mg/mL, 1 mg/mL, 5 mg/mL and 10 mg/mL.

1.4 Preparation of rADAMTS13

The rADAMTS13 (Lot: HR5BK00) in-house reference preparation (277.5 U/mL) was diluted in vortex assay buffer to final rADAMTS13 concentrations of 0.25 U/mL, 0.5 U/mL, 1 U/mL and 2 U/mL.

1.5 Sample Preparation

Each set of experiments contained the following samples: (i) test samples, which consist of VWF cleavage incubation mixture with rADAMTS13 and hemoglobin; (ii) control samples, which consist of VWF cleavage incubation mixture with rADAMTS13 without hemoglobin; and (iii) negative control samples, in which no ADAMTS13-mediated VWF cleavage is expected (resulting in uncleaved VWF). Negative control samples consist of incubation mixture including hemoglobin either without rADAMTS13 or in the presence of rADAMTS13 with addition of 10 mM EDTA to chelate divalent cations and to block ADAMTS13-mediated VWF cleavage.

1.6 Reaction Mixture

Two different experimental set-ups were prepared. Reactions mixtures were incubated in 0.2 mL thin-wall reaction tubes (Order No. 732-0548, VWR; Vienna, Austria).

(i) Pre-Incubation of Platelets, rVWF, and Hemoglobin for 30 Minutes Prior to Addition of Purified rADAMTS13

For the pre-incubation setup, a mixture of purified recombinant human VWF (3 IU/mL) and assay buffer containing reconstituted lyophilized formalin-fixed platelets (300×10³ cells/μL) was pre-incubated with different concentrations of plasma-purified human hemoglobin in a total volume of 45 μL. The respective control samples without hemoglobin were prepared in the same way but instead with the appropriate volume of vortex assay buffer. Pre-incubation of test and control samples was done for 30 minutes under constant vortexing at 2500 rpm at RT on the MixMate vortexer (Order No. 732-6009, Eppendorf; Hamburg, Germany). Pre-incubation was performed for the following experiments: inhibitory hemoglobin (see Section 4.1 of this Example) and pre-incubation versus direct incubation (see Section 4.3 of this Example).

(ii) Direct Incubation of Platelets, rVWF, Hemoglobin, and Purified rADAMTS13 at the Same Time

To investigate whether it makes a difference in the extent of VWF cleavage when hemoglobin is already bound to VWF (and requires displacement from VWF by ADAMTS13) or when hemoglobin competes with ADAMTS13 for VWF binding when reaction components are mixed at the same time, a direct incubation set-up was prepared. For the direct incubation setup, purified recombinant human VWF (3 IU/mL) and assay buffer containing reconstituted lyophilized formalin-fixed platelets (300×10³ cells/μL) was mixed with different concentrations of plasma-purified human hemoglobin in a total volume of 45 μL. The respective control samples without hemoglobin were prepared in the same way but instead with the appropriate volume of vortex assay buffer. No further incubation was done before the addition of rADAMTS13. Direct incubation was performed for the following experiments: overriding hemoglobin (see Section 4.2 of this Example) and pre-incubation versus direct incubation (see Section 4.3 of this Example).

After either pre-incubation or direct incubation, 15 μl of purified rADAMTS13 at different concentrations were added to the reaction mixtures for a total volume of 604. As a negative control for uncleaved VWF, assay buffer without rADAMTS13 was added. Final reaction mixtures were incubated for 60 minutes at RT under constant vortexing at a rotation rate of 2500 rpm on the MixMate vortexer. All reaction mixtures were then stopped by adding EDTA to a final concentration of 10 mM. VWF cleavage fragments (dimeric fragments of 176 kDa and 140 kDa) were separated on NuPage 3-8% Tris-acetate gels under non-reducing conditions.

An overview of the assay reaction mixture setup is depicted in Table 1.

TABLE 1
Overview of Assay Reaction Mixture Setup for
Test and Control Samples for All Experiments

| | All concentrations listed are final concentrations | |
|---|---|---|
| Setup/Sample | Test sample | Control sample |
| Platelets | 300 × 10³ cells/μL | |
| rVWF | 3 IU/mL | |
| Hemoglobin^{a} | 0.1 mg/mL to 10 mg/mL | 0 mg/mL |

| | |
|---|---|
| VAB volume | mixture is brought up to 45 μL with VAB |
| Pre-incubation at RT^{b} | 30 minutes at 2500 rpm constant vortexing |
| rADAMTS13^{a} | 2 U/mL to 0.25 U/mL |
| Total volume | 60 μL |
| Incubation at RT | 60 minutes at 2500 rpm constant vortexing |
| Reaction stop | 10 mM EDTA |

RT: room temperature;
VAB: vortex assay buffer
^{a}These hemoglobin and rADAMTS13 concentrations were not used for all experiments. Specific concentrations used for each experiment are detailed in Section 4 of this Example.
^{b}Direct incubation was performed for the overriding hemoglobin experiment (see Section 4.2 of this Example) and the pre-incubation versus direct incubation experiment (see Section 4.3 of this Example). Direct incubation of the reaction mixture describes when the rVWF, platelets, hemoglobin and rADAMTS13 were incubated at the same time.

### 2. SDS-PAGE and Immunoblot Analysis

2.1 Preparation of the Positive Control for SDS-PAGE/Immunoblot

As a positive control for ADAMTS13-mediated VWF cleavage products, rVWF cleaved by rADAMTS13 under moderate denaturing urea assay conditions (as described in Section 1 of this Example) was applied on each gel for the visualization of the appropriate distinct VWF cleavage fragment after immunoblot analysis.

2.2 Sample Preparation, SDS-PAGE and Immunoblot Detection

Samples were diluted in NuPage 4× lithium dodecyl sulfate (LDS) sample buffer (40% glycerol, 4% LDS, 4% Ficoll-400, 0.8 M triethanolamine-chloride [pH 7.6], 0.025% phenol red, 0.025% coomassie G250, 2 mM EDTA; Order No. NP0007, Invitrogen; Vienna, Austria), loaded onto a NuPage 3-8% Tris-acetate gel (Order No. EA03755BOX, Invitrogen; Vienna, Austria) at a concentration of about 12 ng VWF per lane, and separated under denaturing, non-reducing conditions.

Each gel contained a prestained protein marker, the positive control generated under urea cleavage conditions, at least one reference control sample without hemoglobin, rADAMTS13 test samples with hemoglobin and the negative control sample (reference sample without rADAMTS13 or reference sample incubated with 10 mM EDTA [final concentration]).

After gel electrophoresis (run at 150 volts for approximately 4 hours), proteins were transferred onto a nitrocellulose membrane (iBlotR gel transfer stacks nitrocellulose; Order No. IB3010-01, Invitrogen; Vienna, Austria). As a validity criterion for the blot transfer, the prestained protein standard had to be apparent on the nitrocellulose membrane.

The membrane was blocked with blocking solution for one hour and then incubated overnight at RT with TBST and 0.3% dry milk containing HRP conjugated rabbit anti-human VWF polyclonal antibody (Product Number: PO₂₂₆; Dako Cytomation, Glostrup, Denmark), in a 1:2000 dilution. The blocking solution contained Tris buffered saline (TBS: 20 mM Tris (pH ˜7.4), 0.9% NaCl; Order No. T5912-11, Sigma; Vienna, Austria) with 0.05% Tween 20 (TBST; Order No. 8.22184.0500, Merck; Vienna, Austria) and 3% dry milk (Order No. 170-6404, Bio-Rad Laboratories, Hercules, Calif., USA). The polyclonal antibody visualizes the 176 kDa and to a lesser extent the 140 kDa dimer VWF fragment (Tan et al., Thromb Res; 121(4): 519-26, 2008).

After antibody incubation, blots were washed in TBST and the specific VWF protein bands were detected with an ultra-sensitive enhanced chemiluminescent substrate (Super Signal West Femto maximum sensitivity substrate; Order No. 34095, Thermo Scientific, Austria) for detection of peroxidase activity of bound anti-VWF HRP-conjugated antibodies. Signals were captured using the ChemiDoc Imager camera system (BioRad, Hercules, Calif., USA) that produces digital images of chemiluminescently stained membranes.

A blot was considered valid if the VWF cleavage fragments (i.e., dimers of 176 kDa) of the positive blot control sample generated under urea cleavage conditions was detectable after VWF-specific immunoblotting.

Recorded images were further analyzed by densitometry to evaluate the relative amount of protein staining in the particular cleavage band (described next in Section 3 of this Example).

### 3. Data Analysis

3.1 Image Analysis

The recorded images were opened in the evaluation program of the ChemiDoc Imager camera system and the VWF cleavage products to be analyzed (i.e., the dimer of the 176 kDa fragment) were identified and marked. The color intensity of the marked area per lane, indicated as volume intensity, was then calculated by the evaluation program. These final intensity values were exported to Microsoft Office Excel 2007 for further analysis.

3.2 Control Sample Assessment

For each test sample containing hemoglobin, the respective control sample without addition of hemoglobin was loaded on the gel at least once. In cases where a sample was loaded in duplicate, a mean reference intensity value from the two duplicate intensity values was calculated.

The intensity value of the control sample was set to 100% for the subsequent comparative evaluation with the test samples. For each test sample, the deviation from the intensity value of the control was then determined.

3.3 Methods of Data Analysis

Results are represented as % ratio and were calculated according to the formula:

Ratio [%]=(mean) intensity value_{Test sample}/(mean) intensity value_{Control sample}*100

This calculation was done for each gel separately.

### 4. Results

4.1 Inhibitory Effect of Hemoglobin on ADAMTS13-mediated VWF Multimer Cleavage

The inhibitory effect of hemoglobin on ADAMTS13-mediated VWF multimer cleavage was evaluated for each of the ADAMTS13 concentrations, namely 1 U/mL, 0.5 U/mL and 0.25 U/mL, in the presence of increasing concentrations of hemoglobin (0 mg/mL, 0.5 mg/mL, 1 mg/mL, 5 mg/mL, and 10 mg/mL). Hb concentrations covered the range of plasma Hb observed in SCD patients (20 to 330 μg/mL and >400 μg/mL during vaso-occlusive crises). Normal human plasma concentration of ADAMTS13 is around 1 U/mL.

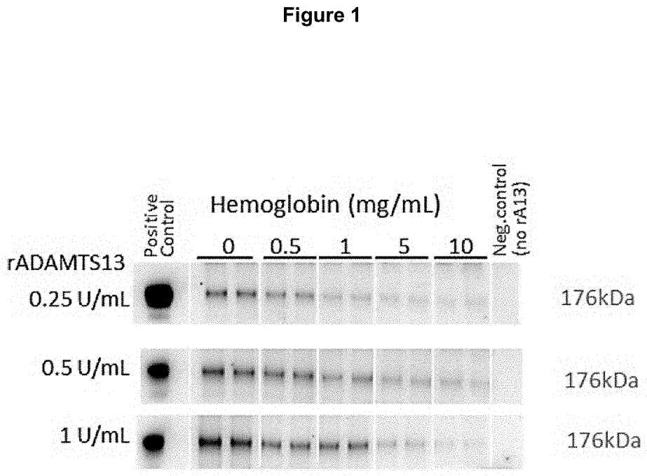

FIG. 1 shows a representative example of the generated dimeric 176 kDa VWF cleavage fragment after incubation of VWF substrate with rADAMTS13 concentrations of 0.25 U/mL, 0.5 U/mL or 1 U/mL in the presence of increasing concentrations of hemoglobin (0.5 mg/mL, 1 mg/mL, 5 mg/mL, and 10 mg/mL) compared to reactions without hemoglobin (0 mg/mL). First visual inspection clearly showed the declining signal of the 176 kDa VWF cleavage fragment with increasing hemoglobin concentrations added to the cleavage reaction at the various rADAMTS13 titers compared to control reactions without addition of hemoglobin. Control reactions with 0.25 U/mL, 0.5 U/mL, and 1 U/mL rADAMTS13 in absence of hemoglobin showed a dose-dependent increase of the 176 kDa dimeric VWF cleavage fragment.

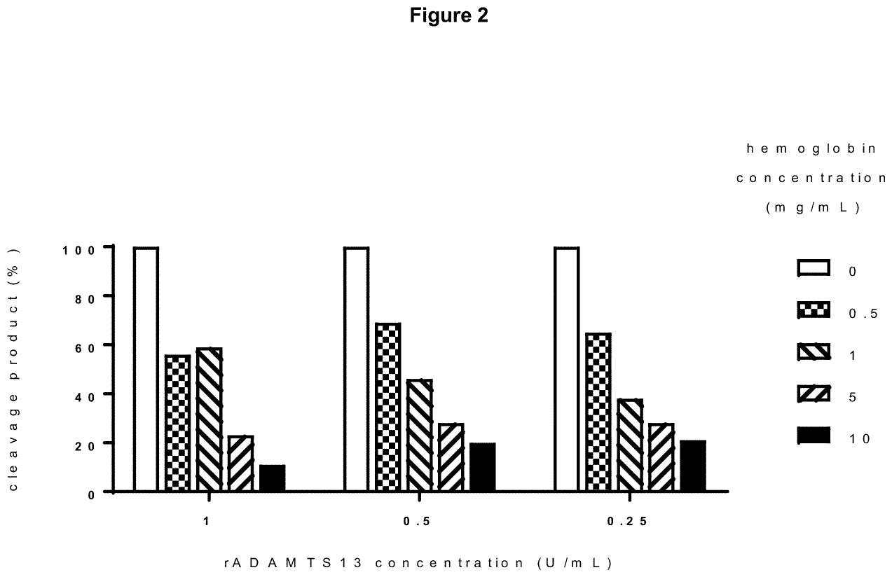

To confirm this visual inspection, densitometric evaluation of the 176 kDa fragment was performed and signal density of the test samples with hemoglobin in relation to the respective controls without hemoglobin was evaluated. Table 2 and FIG. 2 show the densitometric and graphical evaluation of the different rADAMTS13 concentrations (0.25 U/mL, 0.5 U/mL, and 1 U/mL), each incubated with 4 different concentrations of hemoglobin (0.5 mg/mL, 1 mg/mL, 5 mg/mL, and 10 mg/mL). Control samples without addition of hemoglobin were set to 100% and compared to samples of the respective rADAMTS13 concentration with increasing hemoglobin concentrations as described in Section 3.2. Results are represented as % ratio according to the formula described in Section 3.3.

TABLE 2
Densitometric Evaluation of Dimeric 176 kDa VWF
Cleavage Fragment: Evaluation of Inhibitory Effect
of Increasing Concentrations of Hemoglobin

| | | Hemoglobin concentration mg/mL | | | | |
|---|---|---|---|---|---|---|
| rADAMTS13 | | 0 | 0.5 | 1 | 5 | 10 |
| concentration³ | | Percent ratio of the dimeric 176 kDa cleavage product^{b} | | | | |
| 1 | U/mL | 100 | 56 | 59 | 23 | 11 |
| 0.5 | U/mL | 100 | 69 | 46 | 28 | 20 |
| 0.25 | U/mL | 100 | 65 | 38 | 28 | 21 |

^{a}rADAMTS13 concentration based on FRETS-VWF73 activity results
^{b}Ratio [%] = (mean) intensity value of the sample with hemoglobin/(mean) intensity value of the control sample without hemoglobin *100

At 1 U/mL rADAMTS13, stepwise increasing hemoglobin concentrations (0.5 mg/mL, 1 mg/mL, 5 mg/mL, and 10 mg/mL) showed ratios between 59% and 11%. In a similar way, 0.5 U/mL rADAMTS13 led to ratios between 69% and 20%, and for 0.25 U/mL rADAMTS13, ratios between 65% and 21% were found. Results depicted in Table 2 and FIG. 2 confirmed the inhibitory effect of increasing concentrations of hemoglobin on ADAMTS13-mediated VWF cleavage.

4.2 Overriding Effect of rADAMTS13 Concentration on the Inhibitory Effect of Hemoglobin on ADAMTS13-mediated VWF Multimer Cleavage

Visual inspection of the results in the previously described pilot experiment suggests that at constant hemoglobin concentration, increasing concentrations of rADAMTS13 can overcome the interfering effect of hemoglobin on VWF cleavage.

To show in more detail that rADAMTS13, in appropriate concentrations, is able to override the inhibitory effect of hemoglobin on VWF multimer cleavage, the following concentrations for rADAMTS13 were selected: 0.25 U/mL, 0.5 U/mL, 1 U/mL, and 2 U/mL, and the following concentrations for hemoglobin were selected: 0.1 mg/mL, 0.5 mg/mL, and 1 mg/mL.

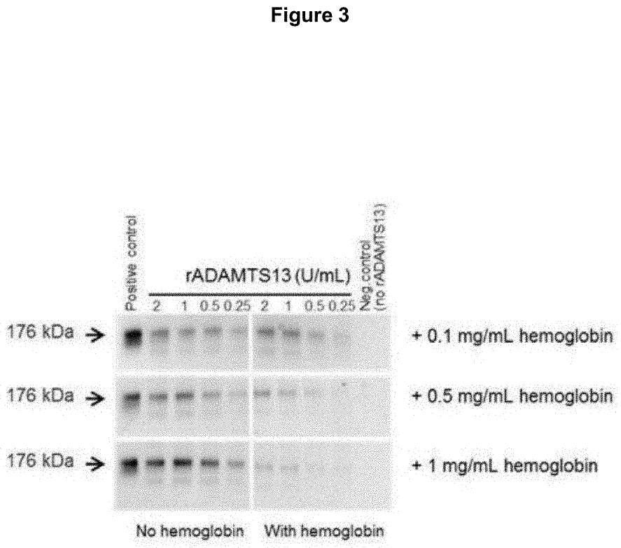

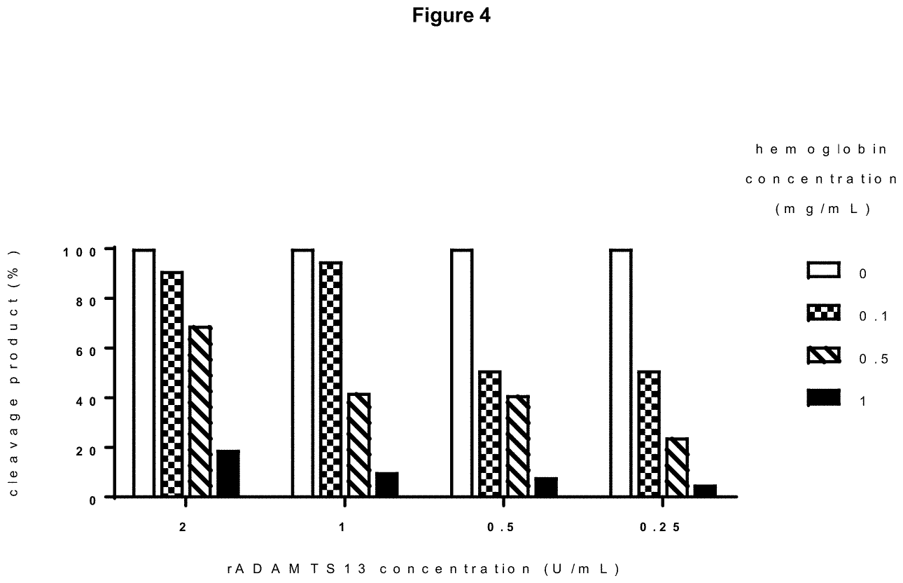

FIG. 3 shows the resulting dimeric 176 kDa VWF cleavage fragments in a VWF-specific immunoblot of samples with constant hemoglobin (0.1 mg/mL, 0.5 mg/mL, and 1 mg/mL) but different rADAMTS13 concentrations (0.25 U/mL, 0.5 U/mL, 1 U/mL, and 2 U/mL) compared to the respective controls without addition of hemoglobin separated on the same immunoblot. The corresponding densitometric and graphical evaluations for the immunoblot shown in FIG. 3 are depicted in Table 3 and FIG. 4. Control samples without hemoglobin were set to 100%, correlated to the respective samples with hemoglobin. Results are represented as % ratio of the dimeric 176 kDa VWF cleavage product.

TABLE 3
Densitometric Evaluation of Dimeric 176 kDa VWF Cleavage
Fragment: Evaluation of the Potential rADAMTS13 Concentration
to Override the Inhibitory Effect of Hemoglobin

| | | Hemoglobin concentration mg/mL | | | |
|---|---|---|---|---|---|
| rADAMTS13 | | 0 | 0.1 | 0.5 | 1 |
| concentration^{a} | | Percent ratio of the dimeric 176 kDa cleavage product^{b} | | | |
| 2 | U/mL | 100 | 91 | 69 | 19 |
| 1 | U/mL | 100 | 95 | 42 | 10 |
| 0.5 | U/mL | 100 | 51 | 41 | 8 |
| 0.25 | U/mL | 100 | 51 | 24 | 5 |

^{a}rADAMTS13 concentration based on FRETS-VWF73 activity results
^{b}Ratio [%] = (mean) intensity value of the sample with hemoglobin/(mean) intensity value of the control sample without hemoglobin *100

At the lowest hemoglobin concentration of 0.1 mg/mL, increasing rADAMTS13 concentrations reconstituted ADAMTS13-mediated VWF cleavage. The extent of reconstitution at 1 U/mL rADAMTS13 was close to the extent of VWF cleavage in the control sample in the absence of hemoglobin. In the presence of 0.5 mg/mL hemoglobin in the cleavage reaction, stepwise increasing rADAMTS13 concentrations gradually enabled VWF cleavage from 24% up to 69% ratio compared to the respective controls lacking hemoglobin. Likewise the dose-dependent overriding effect of rADAMTS13 was observed at 1 mg/mL hemoglobin concentration, albeit at the highest rADAMTS13 concentration of 2 U/mL only about 19% of VWF cleavage fragment was generated compared to cleavage conditions without hemoglobin. These results suggested that at 1 mg/mL hemoglobin, greater than 2 U/mL rADAMTS13 concentrations are needed to overcome hemoglobin interference.

Overall, the results indicate that rADAMTS13 has a dose-dependent potency to overcome the inhibitory effect of hemoglobin on VWF cleavage.

4.3 Effect of Pre-Incubation of Hemoglobin and rVWF Before rADAMTS13 Addition

Experiments were also performed with or without pre-incubation (30 minutes, at constant vortexing at 2500 rpm) of hemoglobin and rVWF before rADAMTS13 was added to the reaction mixture. The objective was to investigate if the accessibility of VWF was influenced when hemoglobin had time to pre-occupy VWF binding sites that are supposed to interfere with ADAMTS13 binding and its cleavage.

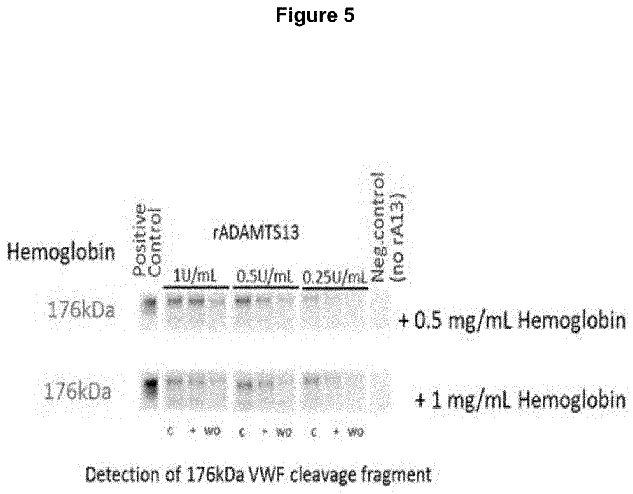

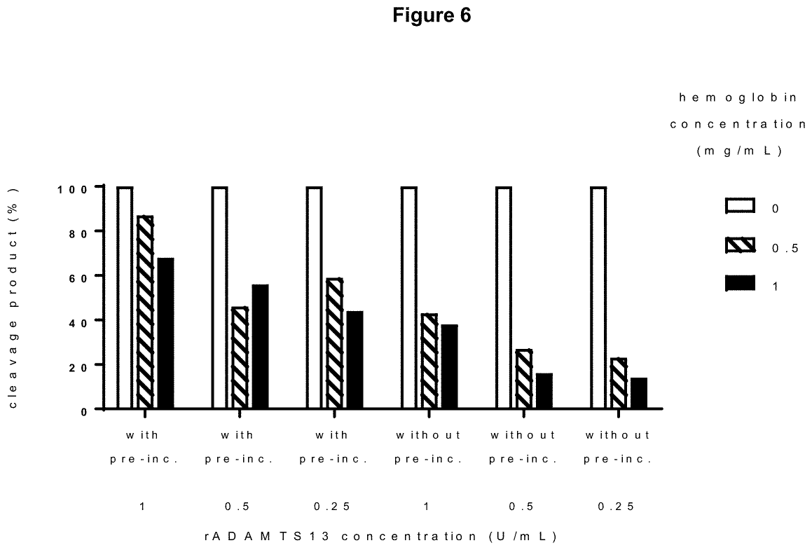

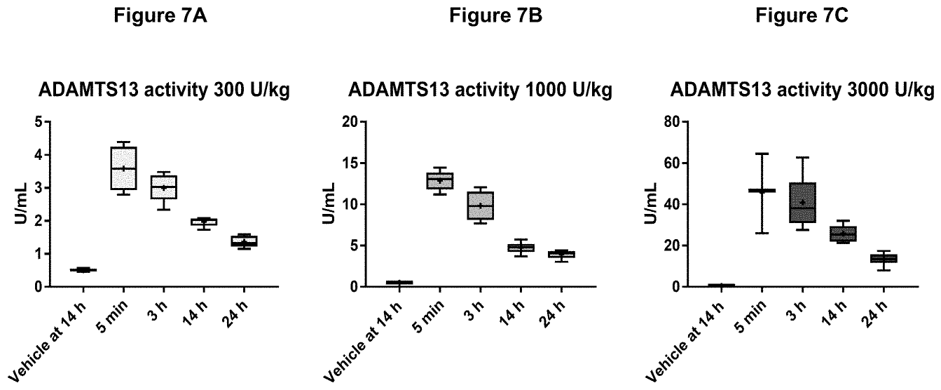

FIG. 5 shows cleavage reactions of three rADAMTS13 concentrations (0.25 U/mL, 0.5 U/mL, and 1 U/mL) performed with or without pre-incubation of 0.5 mg/mL and 1 mg/mL hemoglobin with rVWF. The dimeric 176 kDa cleavage product is visualized by polyclonal anti-VWF antibody HRP conjugate. The corresponding densitometric and graphical evaluations are depicted in Table 4 and FIG. 6.

TABLE 4
Densitometric Evaluation of Dimeric 176 kDa
VWF Cleavage Fragment: Evaluation of Cleavage
Reaction With or Without Pre-incubation

| | | | Hemoglobin concentration mg/mL | | |
|---|---|---|---|---|---|
| | | | 0 | 0.5 | 1 |
| rADAMTS13 | | | Percent ratio of the dimeric 176 kDa | | |
| concentration^{a} | | | cleavage product^{b} | | |
| With | 1 | U/mL | 100 | 87 | 68 |
| pre- | 0.5 | U/mL | 100 | 46 | 56 |
| incubation | 0.25 | U/mL | 100 | 59 | 44 |
| Without | 1 | U/mL | 100 | 43 | 38 |
| pre- | 0.5 | U/mL | 100 | 27 | 16 |
| incubation | 0.25 | U/mL | 100 | 23 | 14 |

^{a}rADAMTS13 concentration based on FRETS-VWF73 activity results
^{b}Ratio [%] = (mean) intensity value of the sample with hemoglobin/(mean) intensity value of the control sample without hemoglobin *100

A dose-dependent inhibition of hemoglobin on rADAMTS13-mediated VWF cleavage was shown with or without pre-incubation of hemoglobin and rVWF. The interfering effect of hemoglobin could be overcome by increasing the rADAMTS13 concentration.

The densitometric evaluation of the dimeric VWF cleavage fragment generated in reaction mixtures with or without pre-incubation of hemoglobin and rVWF revealed the following: (i) rADAMTS13 supplemented after a pre-incubation time of hemoglobin and rVWF was capable to cleave VWF, thus suggesting that hemoglobin that pre-occupied rVWF was competitively replaced by rADAMTS13, and (ii) the extent of rVWF cleavage was different in reaction mixtures with or without pre-incubation.

With pre-incubation, rVWF was cleaved to a higher extent than without pre-incubation: at rADAMTS13 concentration of 0.25 U/mL to 1 U/mL, the ratio of the dimeric 176 kDa cleavage product was between 59% to 87% at 0.5 mg/mL hemoglobin, and between 44% to 68% at 1 mg/mL hemoglobin. In contrast, when rADAMTS13 and hemoglobin was incubated at the same time with rVWF, less rVWF cleavage fragment was generated: at rADAMTS13 concentration of 0.25 U/mL to 1 U/mL, the ratio of the dimeric 176 kDa cleavage product was between 23% to 43% at 0.5 mg/mL hemoglobin, and between 14% to 38% at 1 mg/mL hemoglobin.

These results confirmed the published inhibitory effect of increasing concentrations of hemoglobin on ADAMTS13-mediated cleavage. Hemoglobin concentrations were in the range of extracellular hemoglobin (ECHb) observed in patients during acute sickle cell related events (usually 20-330 μg/mL and >400 μg/mL during vaso-occlusive crises). In addition, it was demonstrated that the inhibitory effect of hemoglobin can be overwhelmed in vitro by appropriate concentrations of rADAMTS13.

### Example 2

### Pharmacokinetic/Pharmacodynamic Study with ADAMTS13 in Tim Townes Mice Testing

The objective of this study was to evaluate the pharmacokinetic profile and efficacy of rADAMTS13 (referred to as SHP655 in this Example) after single intravenous (IV) bolus administration in Tim Townes SS mice, under normal oxygen conditions.

The intravenous (IV) route of administration was selected for this study as this route has been defined as the route of human exposure.

The dose levels were required to study the dose dependency of SHP655 PK/PD. The top dose has already been administered to this strain of mice in a previous study (see Example 7 of International Publication No. WO/2018/027169, which is incorporated herein by reference in its entirety).

### 1. Animal Procedures and Experimental Design

A total of 78 male Tim Townes SS mice were assigned to four study groups as indicated in Table 5. Animals were obtained from Jackson Laboratories (US) or Charles River Laboratories (Sulzfeld, Germany) and the age range at delivery was 4-8 weeks. Subsequent to their arrival at the animal care facility, all animals were subjected to a general physical examination by a qualified member of the veterinary staff to ensure normal health status. Animals were held in quarantine at least 5 days starting at the day of delivery. Animals were housed in isolated ventilated cages (IVC-GM 500) and kept at a targeted temperature of 20-24° C., at a targeted relative humidity of 40-70% and at a light:dark ratio of 1:1 (12 h light: 12 h dark; artificial lighting). Animals were housed in individual cages and cages were changed every second week. Air changes were allowed >60 times per hour. Animals received Ssniff R/M-Haltung diet (Ssniff Spezialdiaten GmbH, Soest, Germany) and water ad libitum. Bedding, nest building materials, and hay were provided (ABEDD Lab and Vet Service GmbH, Vienna, Austria). Body weight was monitoring before start of the study, and daily clinical observation by care staff and clinical signs were recorded. For euthanasia, human endpoints are applied and samples for histopathology were isolated, snap frozen and fixed in 4% phosphate buffered formaldehyde for further analysis. Unscheduled death animals were necropsied if possible or required. Animals were euthanized using overdose of pentobarbital or under deep anesthesia by cervical dislocation.

The animals received individual numbers and were marked with indelible ink according to the marking scheme shown in Table 5.

TABLE 5
Study Groups, Animal Number, and O₂ concentration

| | | Dose | Animals per | Animal |
|---|---|---|---|---|
| Group | Test items | (IU/kg) | Group | Number |
| A | Buffer^{a} | NA | 6 | 1-6 |
| B | SHP655 | 300 | 24 | 7-30 |
| C | SHP655 | 1000 | 24 | 31-54 |
| D | SHP655 | 3000 | 24 | 55-78 |

NA: not applicable
^{a}SHP655 buffer consists of 2 mM calcium chloride, 20 mM L-histidine, 3% mannitol, 1% sucrose, 0.05% polysorbate 80, pH 6.9-7.1

### 2. Preparation of Test Article and Control Item

The test article and control item for Groups A through D were prepared freshly on the day of injection. Lyophilized SHP655 (stored frozen) was allowed to reach room temperature. The test article contained rADAMTS13 in the formulation buffer (calcium chloride (2 mM), L-histidine (20 mM), mannitol (3% w/w), sucrose (1% w/w), and polysorbate 80 (0.05% w/w), pH 6.9-7.1). The control item contained the formulation buffer of SHP655. SHP655 was reconstituted in 5 mL of sterile water for injection (Lot No. VN549058, Baxalta Innovations GmbH). After reconstitution, SHP655 was kept at room temperature for at least one minute and then gently swirled to ensure complete dissolution. For injection, the reconstituted SHP655 was diluted with the formulation buffer for SHP655 (Table 6). The buffer for SHP655 (vehicle) was injected as control. After completion, the formulations were gently mixed by slow inversion. The final dilutions were supplied in a box with wet ice in labeled tubes (study number/group/dose) filled with the appropriate volume for treatment of the corresponding group. Final dilutions were kept on ice and applied to animals within 3 hours.

TABLE 6
Formulation of Test Article (e.g., for a mouse at 30 g weight)

| | | | Dose | Dose | Total | Stock | Formulation |
|---|---|---|---|---|---|---|---|
| | | Dose | Volume | Conc | Volume | SHP655^{a} | Buffer |
| Group | Treatment | (IU/kg) | (mL/kg) | (IU/mL) | (μL) | (μL) | (μL) |
| A | Vehicle | NA | 10 | NA | 300 | NA | 300 |
| B | SHP655 | 300 | 10 | 30 | 300 | 24.7 | 275.3 |
| C | SHP655 | 1000 | 10 | 100 | 300 | 82.4 | 217.6 |
| D | SHP655 | 3000 | 10 | 300 | 300 | 247.3 | 52.7 |

Conc: concentration;
NA: not applicable
^{a}Stock SHP655 concentration is 364 IU/mL.

### 3. Administration of SHP655

The test article and control item were injected in conscious restrained animals on one occasion via a lateral tail vein, and was based upon the individual animal's latest body weight recorded a day before injection. The administration volume was 10 mL/kg. Pre- and post-dose formulation samples (100 μL aliquots) were stored deep-frozen (<−60° C.).

The day of dosing was designated as Day 0. After dosing, the animals were monitored and findings were recorded as described in Section 1 of this Example.

### 4. Blood Sampling

Blood was sampled (n=6 per timepoint) at 0.083 (5 minutes), 3, 14 and 24 hours after drug administration, while only at 14 hours after the vehicle administration.

4.1 Retro-Orbital Puncture

Retro-orbital blood (0.3 mL EDTA blood) was collected only from the mice which were sacrificed at 14 hours after administration.

Immediately prior to blood collection, animals were anaesthetized with isoflurane (Lot No. 6065016, Intervet, Austria) using UNO-Univentor 400 anesthesia unit. The animal was then restrained by grasping a neck skin fold and a glass capillary was carefully inserted into the plexus adjacent to the median corner of the eye. The capillary was gently rotated behind the eye until blood begins to flow through the capillary. The glass capillary was then pulled out of the eye and the blood drops were collected in a clearly labeled EDTA tube (Lot No. 160805, Greiner Bio-one). After reaching the desired volume, the neck hold was relaxed and the bleeding was stopped by gently pressing a sterile cotton pad on the eye. The tube was capped and the sample was gently mixed by slow inversion.

4.2 Cardiac Puncture

A terminal cardiac puncture blood (0.5-0.7 mL blood) was collected for analysis of ADAMTS13 activity (all time points: 0.083, 3, 14 and 24 hours) and some parameters listed in Section 7 of this Example.

For this purpose, animals were anaesthetized (approximately 100-150 mg Ketasol [Ketamine hydrochloride; Lot No. 6680115, OGRIS Pharma GmbH]+10-20 mg Rompun® [Xylazine hydrochloride; Lot No. KPOAGNA, Bayer, Germany] diluted with sodium chloride [Lot No. 19HL27WB, Fresenius, Austria] at a volume of 10 mL/kg) and blood was collected with 2 mL syringes fitted with a 25G needle without opening the thorax or puncturing the liver. The blood was withdrawn slowly and carefully to prevent circulatory/cardiac collapse. The needle was then removed from the syringe before transferring the sample into an individually labeled lithium heparin tube (Lot No. 7071511, Sarstedt). The tubes were capped and then the samples were gently mixed by slow inversion. The lithium heparin blood was used for plasma preparation.

4.3 Preparation of Heparin Plasma

All heparinized blood samples were centrifuged as soon as possible. Heparin blood samples were centrifuged at 2200 g for 10 minutes at room temperature. The supernatant plasma was transferred with a plastic pipette into a second clean and clearly labeled Eppendorf tube. When transferring the supernatant into the second Eppendorf tube, caution was taken to avoid withdrawing any cells from the “buffy coat” with the plasma. A second centrifugation (plasma supernatant) was performed (2200 g for 5 minutes at room temperature). The plasma was again carefully removed with a plastic pipette (no cells from sediment) into a clearly labeled Eppendorf tube. At all timepoints, plasma was analyzed for ADAMTS13 activity and some parameters in Section 7 of this Example.

### 5. Analysis of ADAMTS13 Activity

ADAMTS13 activity of all heparin plasma samples was analyzed with the FRETS-VWF73 assay. In brief, FRETS-VWF73 is a fluorescence quenching substrate for ADAMTS13. It is a peptide consisting of 73 amino acids of the A2 domain of human VWF (D1596-R1668), including the cleavage site of ADAMTS13 (Y1605-M1606). The fluorescent signal of uncleaved FRETS-VWF73 is quenched by the quencher via fluorescence resonance energy transfer between the fluorophore and the quencher. Cleavage of FRETS-VWF73 substrate by ADAMTS13 results in a fluorescent signal caused by the spatial distance between fluorophore and quencher. The fluorophore is excited at 340 nm and emits light at 450 nm. Plasma samples were diluted in the sample dilution buffer to an estimated ADAMTS13 activity of 80 mU/mL to 5 mU/mL. Diluted standard (normal human plasma with a defined ADAMTS13 activity of 1 U/mL), controls and plasma samples (all added at 100 μL per well) is mixed with 100 μL per well of FRETS-VWF73 substrate in a 96 well microplate to start the cleavage reaction of FRETS-VWF73 and ADAMTS13. This process is measured with a fluorescence spectrophotometer every 5 minutes over a period of one hour. The increase of the signal over this period of time corresponds to ADAMTS13 activity in the sample.

### 6. Isolation of Organs

Selected organs (e.g., lung, kidney, spleen and liver) from all groups were isolated (at the 14 hour timepoint only). For all groups, one part was fixed in appropriated solution (e.g., 4% phosphate buffered formaldehyde), and the other part was frozen in liquid nitrogen.

Fixed tissue (lung, kidney and liver) in 4% phosphate buffered formaldehyde (Lot No. 16B160010, VWR Chemicals PROLABO) was sent for histopathology analysis. Fresh frozen tissue (lung, kidney and liver) in liquid nitrogen (in 2 mL Eppendorf tubes) were sent to NMI TT (Reutlingen, Germany) for exploratory analysis.

### 7. Additional Parameters

The following parameters were analyzed: VWF activity levels, VWF antigen levels, VWF multimers, VWF cleavage fragments, and free hemoglobin levels.

7.1 VWF Activity Assay

The VWF:CBA was performed according to the product leaflet of ZYMUTEST VWF:CBA (manufactured by Hyphen BioMed, 155, rue d′Eragny, F95000 Neuville-sur-Oise, France).

In a first step, the diluted calibrator, controls and samples were introduced into a micro-well coated with fibrillar collagen (equine, type 1 and 3). When present, VWF was captured onto the solid phase through its collagen binding activity. Following a washing step, the immunoconjugate, which is a polyclonal antibody coupled to horse radish peroxidase (HRP), was added and allowed to bind to free epitopes of immobilized VWF. Following a washing step, the peroxidase substrate, 3,3′,5,5′-tetramethylbenzidine (TMB), in presence of hydrogen peroxide (H₂O₂), was applied and a blue color developed. When the reaction was stopped with sulfuric acid, a yellow color was obtained. The amount of color developed was directly proportional to the concentration of human VWF:CBA in the tested sample.

7.2 VWF Antigen Assay

The assay was performed according to the product leaflet of ASSERACHROM VWF:Ag (Diagnostica Stago, Asnieres sur Seine, France).

In brief, VWF was captured by rabbit polyclonal anti-human VWF:Ag antibody pre-coated on the wells of a plastic microplate well. Next, rabbit anti-human VWF antibodies coupled with peroxidase bind to the free antigenic determinants of the bound VWF. The bound enzyme peroxidase was revealed by its action on the TMB substrate. After stopping the reaction with 0.5 N Sulfuric acid, the intensity of the color was directly proportional to the concentration of VWF initially present in the sample.

7.3 VWF Multimer Assay

The multimeric structure of VWF was analyzed by horizontal SDS agarose gel electrophoresis. Low resolution (1% agarose) conditions were used to analyze the size distribution of VWF multimers. Samples were diluted based on their VWF:Ag content and incubated with Tris-EDTA-SDS buffer. The multimers were then separated under non-reducing conditions on an agarose gel. The VWF multimers were visualized by immunodetection with a polyclonal rabbit anti-human VWF antibody followed by HRP-conjugated goat anti-rabbit IgG using the chemiluminescence detection kit (Clarity Western ECL) from Bio-Rad (Richmond, Calif., US). VWF multimers were visualized with a CCD-camera and the number of VWF multimers countable by the naked eye was recorded.

7.4 Free Hemoglobin Assay

Free human hemoglobin was analyzed in plasma samples by a commercial sandwich enzyme-linked immunosorbent assay (ELISA) provided by Abcam (ab157707). The range of the in vitro assay for human hemoglobin was between 3.13 ng/mL and 200 ng/mL, the sensitivity was 0.845 ng/mL and the precision below 10%.

In this assay the hemoglobin present in plasma samples reacted with the anti-hemoglobin antibodies which had been adsorbed to the surface of polystyrene microtiter wells. After the removal of unbound proteins by washing, anti-hemoglobin antibodies conjugated HRP, were added. These enzyme-labeled antibodies form complexes with the previously bound hemoglobin. Following another washing step, the enzyme bound to the immunosorbent was assayed by the addition of a chromogenic substrate, 3,3′,5,5′-TMB. The quantity of bound enzyme varied directly with the concentration of hemoglobin in the sample tested; thus, the absorbance, at 450 nm, was a measure of the concentration of hemoglobin in the test sample. The quantity of hemoglobin in the test sample can be interpolated from the standard curve constructed from the standards, and corrected for sample dilution.

### 8. Statistical Analysis

The statistical analysis was performed with GraphPad PrismVersion 7.03.

The pharmacokinetic analysis was performed with Phoenix WinNonlin version 6.3 (Pharsight). Pharmacokinetic data were assessed using a sparse sampling design, i.e., a serial sampling design where only one sample is taken per animal at one of the timepoints investigated.

Pharmacokinetic parameters for SHP655 activity concentrations were calculated using the non-compartmental approach.

The mean baseline concentration values of SHP655 (measured in Group A) were subtracted from each plasma concentration measured in Groups B, C and D (see Table 10).

Concentrations that gave the maximum mean concentration across all time points were used as estimates for maximum concentration following infusion (Cₘₐₓ) and summarized by the arithmetic mean. The minimum time observed to reach the Cₘₐₓ concentration was defined as Tₘₐₓ. The area under the concentration versus time curve from 0 to the last sampling time point with a quantifiable concentration, the total area under the concentration versus time curve, the terminal half-life, the mean residence time, the total clearance, the volume of distribution at steady state, and the incremental recovery (calculated as Cₘₐₓ/dose) were also calculated. The actual dose in U/kg was used for the calculations.

### 9. Results

9.1 Dose Solution Analysis

The results of the dose solution analysis are reported in Table 7.

TABLE 7
Dose Solution Analysis

| | Theoretical | Theoretical | Measured | Actual |
|---|---|---|---|---|
| | Dose | Concentration | Concentration | Dose |
| Group | (IU/kg) | (IU/mL) | (U/mL) | (U/kg) |
| B | 300 | 30 | 32.5 | 325 |
| C | 1000 | 100 | 116.0 | 1160 |
| D | 3000 | 300 | 310.3 | 3103 |

9.2 Age, Body, and Organ Weight

The mean body weight and age range are reported in Table 8.

TABLE 8
Mean Body Weight and Age of the Animals

Body Weight (g)

| | Animal | | Standard | Age Range |
|---|---|---|---|---|
| Group | Numbers | Mean | Deviation | (months) |
| A | 6 | 33.9 | 3.0 | 4.4 |
| B | 24 | 32.4 | 2.3 | 3.7-5.1 |
| C | 24 | 32.6 | 2.6 | 4.0-4.9 |
| D | 24 | 33.3 | 2.7 | 4.0-4.4 |

Body and organs weight of the animals sacrificed at 14 hours after administration are reported in Table 9.

TABLE 9
Body and Organ Weights

| | Liver | | Liver | | Spleen | | Spleen | | Kidney | |
|---|---|---|---|---|---|---|---|---|---|---|
| Group/ | (g) | | (% of body) | | (g) | | (% of body) | | (g) | |
| Treatment | Mean | SD | Mean | SD | Mean | SD | Mean | SD | Mean | SD |
| A/ | 2.70 | 0.17 | 7.98 | 0.55 | 1.87 | 0.26 | 5.50 | 0.53 | 0.711 | 0.262 |
| Vehicle | | | | | | | | | | |
| B/ | 2.73 | 0.42 | 7.93 | 0.96 | 1.81 | 0.22 | 5.26 | 0.55 | 0.522 | 0.058 |
| 300 IU/kg | | | | | | | | | | |
| SHP655 | | | | | | | | | | |
| C/ | 2.82 | 0.29 | 8.29 | 0.60 | 1.81 | 0.32 | 5.38 | 1.19 | 0.567 | 0.057 |
| 1000 IU/kg | | | | | | | | | | |
| SHP655 | | | | | | | | | | |
| D/ | 2.41 | 0.25 | 7.44 | 0.41 | 1.67 | 0.23 | 5.20 | 0.90 | 0.562 | 0.057 |
| 3000 IU/kg | | | | | | | | | | |
| SUP655 | | | | | | | | | | |

| | Kidney | | Lung | | Lung | | Body | |
|---|---|---|---|---|---|---|---|---|
| Group/ | (% of body) | | (g) | | (% of body) | | (g) | |
| Treatment | Mean | SD | Mean | SD | Mean | SD | Mean | SD |
| A/ | 2.09 | 0.73 | 0.419 | 0.05 | 1.24 | 0.17 | 33.9 | 3.0 |
| Vehicle | | | | | | | | |
| B/ | 1.52 | 0.15 | 0.411 | 0.059 | 1.21 | 0.21 | 34.3 | 1.6 |
| 300 IU/kg | | | | | | | | |
| SHP655 | | | | | | | | |
| C/ | 1.67 | 0.21 | 0.444 | 0.078 | 1.30 | 0.20 | 34.1 | 2.7 |
| 1000 IU/kg | | | | | | | | |
| SHP655 | | | | | | | | |
| D/ | 1.74 | 0.13 | 0.408 | 0.041 | 1.26 | 0.08 | 32.3 | 2.0 |
| 3000 IU/kg | | | | | | | | |
| SHP655 | | | | | | | | |

SD: standard deviation; n=6 animals per group from the 14 hour timepoint

9.3 Clinical Symptoms and Mortality

Animal B9 died before dosing and was not replaced.

After dosing, no clinical symptoms were observed for all the mice, except for Animal C36, which died just after dosing at 1000 IU/kg (before the 5 minutes sampling).

Spontaneous deaths are not rare in this strain of mice, due to their sickness (Ryan et al., Science; 278(5339): 873-6, 1997). Spontaneous deaths have been observed in-house during the age increase which is accompanied by an increase in the disease state (mice have been supplied at the age of 1-2 months and have been grown in-house till the age of experiments, which is 4-5 months).

9.4 ADAMTS13 Activity

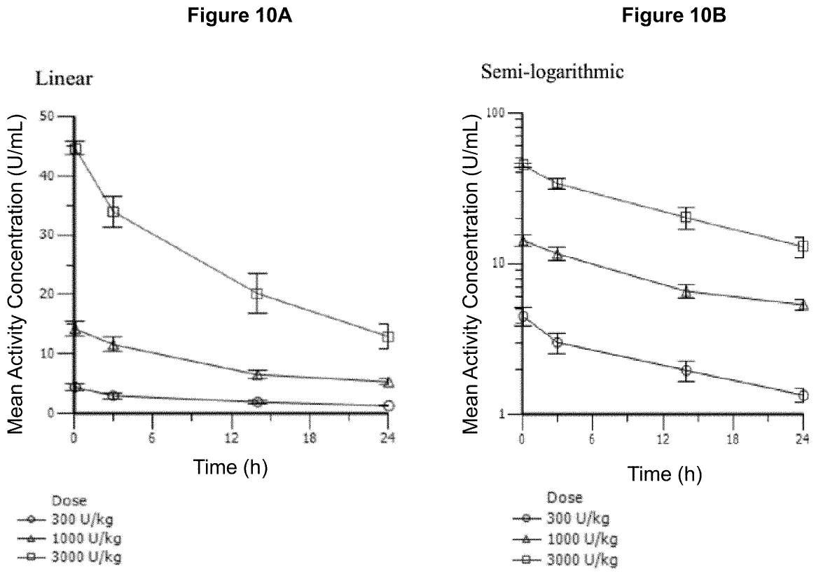

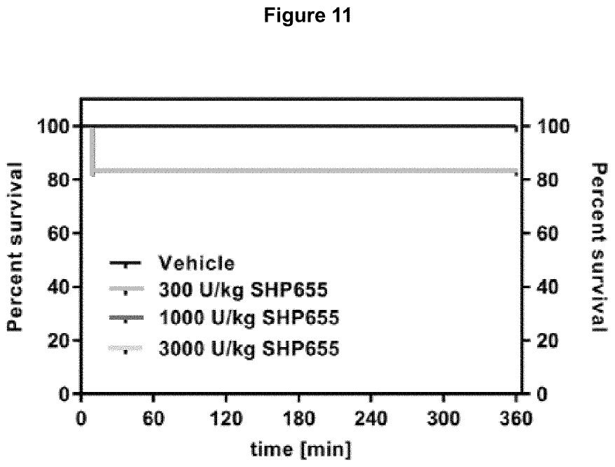

ADAMTS13 activity is reported in Table 10 and also shown in FIGS. 10A-10B.

TABLE 10
ADAMTS13 Activity

| | | | | | Concentration |
|---|---|---|---|---|---|
| | | | | | without |
| | Dose | Time | Animal | Concentration | background |
| Group | (IU/kg) | (h) | Numbers | (U/mL) | (U/mL) |
| A | 0 | 14 | 1 | 0.522 | 0.469^{a} |
| | | | 2 | 0.466 | |
| | | | 3 | 0.435 | |
| | | | 4 | 0.534 | |
| | | | 5 | 0.469 | |
| | | | 6 | 0.388 | |
| B | 300 | 0.083 | 7 | 5.307 | 4.838 |
| | | | 8 | 5.234 | 4.765 |
| | | | 9 | No Sample | No Sample |
| | | | 10 | 0.175^{b} | −0.294^{b} |
| | | | 11 | 5.286 | 4.817 |
| | | | 12 | 4.008 | 3.539 |
| | | 3 | 13 | 3.429 | 2.960 |
| | | | 14 | 3.707 | 3.238 |
| | | | 15 | 3.572 | 3.103 |
| | | | 16 | 3.647 | 3.178 |
| | | | 17 | 3.957 | 3.488 |
| | | | 18 | 2.561 | 2.092 |
| | | 14 | 19 | 2.040 | 1.571 |
| | | | 20 | 2.650 | 2.181 |
| | | | 21 | 2.112 | 1.643 |
| | | | 22 | 2.805 | 2.336 |
| | | | 23 | 2.517 | 2.048 |
| | | | 24 | 2.480 | 2.011 |
| | | 24 | 25 | 1.752 | 1.283 |
| | | | 26 | 1.617 | 1.148 |
| | | | 27 | 1.967 | 1.498 |
| | | | 28 | 1.946 | 1.477 |
| | | | 29 | 1.878 | 1.409 |
| | | | 30 | 1.712 | 1.243 |
| C | 1000 | 0.083 | 31 | 14.549 | 14.080 |
| | | | 32 | 16.322 | 15.853 |
| | | | 33 | 15.482 | 15.013 |
| | | | 34 | 13.914 | 13.445 |
| | | | 35 | 13.473 | 13.004 |
| | | | 36 | No Sample | No Sample |
| | | 3 | 37 | 11.578 | 11.109 |
| | | | 38 | 10.588 | 10.119 |
| | | | 39 | 12.616 | 12.147 |
| | | | 40 | 14.089 | 13.620 |
| | | | 41 | 11.418 | 10.949 |
| | | | 42 | 12.260 | 11.791 |
| | | 14 | 43 | 8.101 | 7.632 |
| | | | 44 | 6.457 | 5.988 |
| | | | 45 | 7.109 | 6.640 |
| | | | 46 | 7.276 | 6.807 |
| | | | 47 | 6.076 | 5.607 |
| | | | 48 | 7.146 | 6.677 |
| | | 24 | 49 | 5.660 | 5.191 |
| | | | 50 | 5.803 | 5.334 |
| | | | 51 | 6.132 | 5.663 |
| | | | 52 | 5.264 | 4.795 |
| | | | 53 | 5.608 | 5.139 |
| | | | 54 | 6.439 | 5.970 |
| D | 3000 | 0.083 | 55 | 45.513 | 45.044 |
| | | | 56 | 45.848 | 45.379 |
| | | | 57 | 45.577 | 45.108 |
| | | | 58 | 45.503 | 45.034 |
| | | | 59 | 45.149 | 44.680 |
| | | | 60 | 42.909 | 42.440 |
| | | 3 | 61 | 29.288 | 28.819 |
| | | | 62 | 35.688 | 35.219 |
| | | | 63 | 35.762 | 35.293 |
| | | | 64 | 34.162 | 33.693 |
| | | | 65 | 36.816 | 36.348 |
| | | | 66 | 34.743 | 34.274 |
| | | 14 | 67 | 24.901 | 24.432 |
| | | | 68 | 22.931 | 22.462 |
| | | | 69 | 19.135 | 18.666 |
| | | | 70 | 22.280 | 21.811 |
| | | | 71 | 18.752 | 18.283 |
| | | | 72 | 15.882 | 15.413 |
| | | 24 | 73 | 11.166 | 10.697 |
| | | | 74 | 15.194 | 14.725 |
| | | | 75 | 12.402 | 11.933 |
| | | | 76 | 11.329 | 10.860 |
| | | | 77 | 15.473 | 15.004 |
| | | | 78 | 15.033 | 14.564 |

^{a}Baseline background (0.469 U/mL) comes from the mean concentrations of Group A animals. It is subtracted from each animal's concentration to give the concentration without background result for each animal.
^{b}Data for Animal B10 was not considered for pharmacokinetic analysis, because there was very likely a misdosing for this animal.

9.5 Pharmacokinetics

The plasma concentration versus time profile for SHP655 is reported in FIGS. 10A and 10B. Pharmacokinetic parameters for SHP655 are reported in Table 11.

TABLE 11
Summary of Pharmacokinetic Parameters for SHP655

| Dose | AUC₀₋ₜ | AUC_{0-inf} | Cₘₐₓ | t½ | MRT | IR^{a} | CL | Vss |
|---|---|---|---|---|---|---|---|---|
| (IU/kg) | (h*U/mL) | h*U/mL) | (U/mL) | (h) | (h) | (kg/mL) | (mL/h/kg) | (mL/kg) |
| 300 | 55.2 | 90.1 | 4.489 | 18.0 | 25.1 | 0.01381 | 3.6 | 90.4 |
| 1000 | 198.5 | 342.2 | 14.279 | 18.6 | 26.8 | 0.01231 | 3.4 | 90.8 |
| 3000 | 581.7 | 864.4 | 44.614 | 15.1 | 21.0 | 0.01438 | 3.6 | 75.5 |

AUC_{0-inf}: total area under the plasma concentration versus time curve; AUC₀₋ₜ: area under the concentration versus time curve from 0 to the last sampling time point (24 hours); CL: total clearance; Cₘₐₓ: maximum concentration following infusion; IR: incremental recovery; MRT: mean residence time; t^{1/2}: terminal half-life; Vₛₛ: volume of distribution at steady state ^{a}IR is calculated as Cₘₐₓ/actual dose. Actual dose for 300 IU/kg is 325 IU/kg, for 1000 IU/kg is 1160 IU/kg, and for 3000 IU/kg is 3103 IU/kg.

The AUC exposure was linearly dose dependent. Clearance is low and the half-life is long. Volume of distribution at steady state is low, but above plasma volume, indicating that SHP655 distributes to other tissues in addition to plasma. PK parameters have to be considered with caution due to the limited study design (the percentage of extrapolated AUC was 32-42%).

9.6 VWF Activity/Antigen and Plasma Hemoglobin Concentration

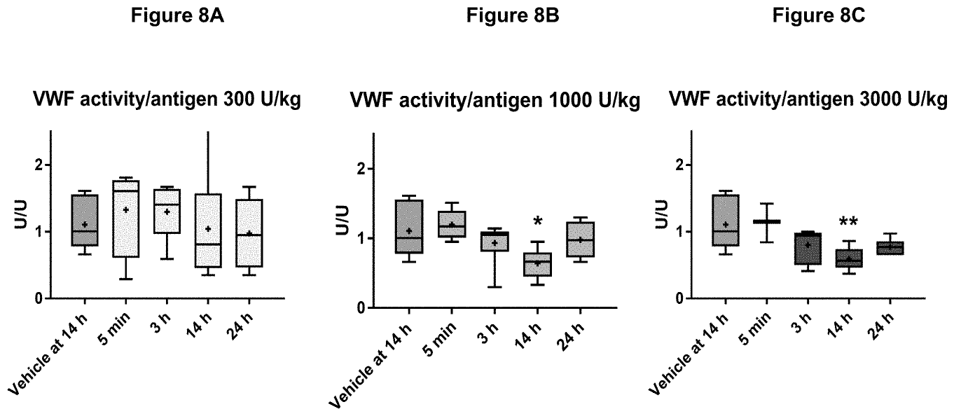

VWF activity and antigen level were determined using the ZYMUTEST VWF:CBA activity assay and the ASSERACHROM VWF:Ag ELISA. VWF activity/antigen ratio is shown in FIGS. 8A-8C.

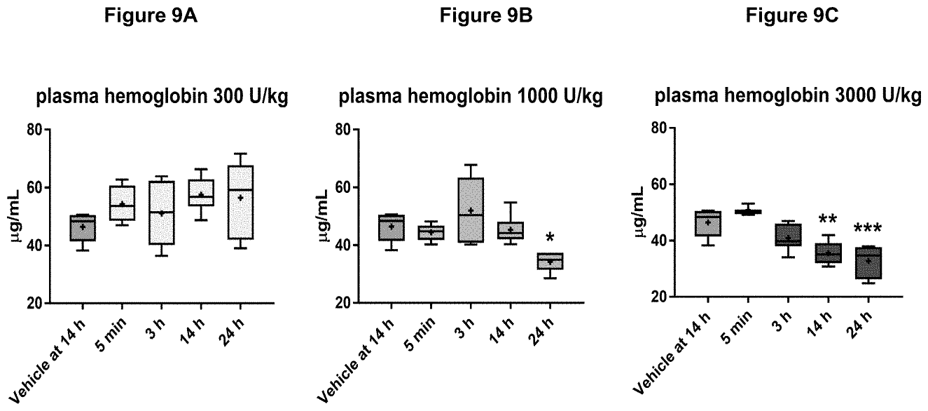

Free hemoglobin was analyzed in plasma samples using a commercial ELISA according to manufacturer's instructions. Plasma hemoglobin concentration is shown in FIGS. 9A-9C.

9.7 Histopathology

Liver, kidney and lung from animals sacrificed at 14 hours were analyzed.

Sections of liver were characterized by the presence of focal to multifocal coalescing areas of coagulative necrosis of minimal to moderate severity, which tended to be near portal triads. In some areas, mixed inflammation (plasma cells, lymphocytes, mononuclear cells, occasional neutrophils and eosinophils) was associated with necrosis, while in other areas this inflammation was isolated in adjacent parenchyma away from areas of necrosis. Sometimes areas of coagulative necrosis were present with no associated inflammation. Golden brown pigment (interpreted as bile or hemosiderin) was present in or near areas of necrosis and inflammation. In other areas this golden brown pigment was present in individual hepatocytes (i.e., bile stasis). Liver sections also contained blood vessels (most likely the portal veins) that were packed with erythrocytes to the extent that it was difficult to distinguish single erythrocytes (also more consistent with hemostasis than congestion). There was diffuse involvement of the blood vessels also in some mice, while in other mice only 3 to 4 blood vessels were involved, which tended to be in the portal triad areas, but in all mice sparing central veins. There was no difference in the severity of these findings across the various different groups.

Diffuse hepatocellular cytomegaly/karyomegaly (a non-specific feature common observed in genetically-modified mice) as well were observed in livers from several mice.

The kidneys of several mice from all groups showed congestion of blood vessels at the cortico-medullary junction. This congestion was observed at mild to moderate grades of severity in mice receiving 0 or 300 IU/kg SHP655, while in mice receiving 1000 or 3000 IU/kg SHP655, the severity was minimal to mild with those receiving 3000 IU/kg SHP655 showing essentially all minimal severity (including one mouse with no congestion). This suggests some treatment effect at higher doses of SHP655 in this study.

All remaining kidney and lung findings were typical background findings commonly observed in laboratory mice.

In comparison to the vehicle group, SHP655-treated Tim Townes SS mice showed a significant decrease of VWF activity/antigen ratio at intermediate and high doses (p<0.05) at 14 hours after ADAMTS13 administration. These results are in line with the proposed mechanism of action of SHP655 in SCD suggesting a decrease of the concentration of ultra-large VWF multimers.

In comparison to the vehicle group, SHP655-treated Tim Townes SS mice showed a significant decrease of free hemoglobin concentration at intermediate and high doses (p<0.05) at 24 hours after ADAMTS13 administration.

Together with the ADAMTS13 activity data, these results indicate that there is a delay in the pharmacodynamic response to SHP655. The maximum effect of SHP655 is not at the maximum plasma concentration of ADAMTS13 (5 minutes after administration), but at 14 hours for VWF activity/antigen ratio and 24 hours for free hemoglobin concentration.

### Example 3

### Efficacy Study of ADAMTS13 in a Sickle Cell Disease Animal Model

The objective of this exploratory efficacy study was to investigate the dose dependent efficacy of recombinant ADAMTS13 (referred to in this Example as SHP655) after intravenous administration of SHP655 in Tim Townes mice, under hypoxic conditions.

The SHP655 efficacy was investigated in Tim Townes mice at 7.0% oxygen.

Similarly to Example 2, the intravenous route of administration has been selected for this study as this route has been defined as the route of human exposure. The dose levels of 300, 1000, and 3000 IU/kg SHP655 were selected based on a previous study (see Example 7 of International Publication No. WO/2018/027169, which is incorporated herein by reference in its entirety) and to reveal a dose dependent effect of SHP655.

### 1. Animal Procedures and Study Design

A total of 24 male Tim Townes SS mice (Homozygous for Hbb^{tm2(HBG1,HBB*)Tow}, Homozygous for Hba^{tm1(HBA)Tow}) were purchased from Jackson Laboratories (US) and obtained via two different shipments where all animals entered the scheduled in-life phase at comparable body weight and age. The age range at delivery was 4-8 weeks. Subsequent to their arrival at the animal care facility, all animals were subjected to a general physical examination by a qualified member of the veterinary staff to ensure normal health status. Animals were held in quarantine at least 5 days starting at the day of delivery. Animals were housed in isolated ventilated cages (IVC-GM 500) and kept at a targeted temperature of 20-24° C., at a targeted relative humidity of 40-70% and at a light: dark ratio of 1:1 (12 h light: 12 h dark; artificial lighting). 1-3 animals were housed per cage and cages were changed every week. Air changes were allowed >60 times per hour. Animals received Ssniff R/M-Haltung diet (Ssniff Spezialdiaten GmbH, Soest, Germany) and water ad libitum. Bedding, nest building materials, and hay were provided (ABEDD Lab and Vet Service GmbH, Vienna, Austria). Weight of the animals was monitoring once weekly starting at the day of delivery, and daily clinical observation by care staff and clinical signs were recorded. For euthanasia, human endpoints are applied and samples for histopathology were isolated, snap frozen and fixed in 4% phosphate buffered formaldehyde (Lot No. 18F090001, VWR International) for further analysis. Unscheduled death animals were necropsied if possible or required. Animals were euthanized using overdose of ketamine/xylazine (OGRIS Pharma GmbH) or under deep anesthesia by cervical dislocation.

The animals received individual numbers and were marked with indelible ink according to the marking scheme shown in Table 12.

TABLE 12
Study Groups, Animal ID, and O₂ concentration

| | Test | Dose | Animals/ | | |
|---|---|---|---|---|---|
| Group | items | (IU/kg) | group | Animal ID | O₂ concentration |
| E | vehicle | n/a | 6 | 33-38 | 5 hours at 7.0% |
| | | | | | and 1 hour at 21% |
| F | SHP655 | 300 | 6 | 39-44 | 5 hours at 7.0% |
| | | | | | and 1 hour at 21% |
| G | SHP655 | 1000 | 6 | 45-50 | 5 hours at 7.0% |
| | | | | | and 1 hour at 21% |
| H | SHP655 | 3000 | 6 | 51-56 | 5 hours at 7.0% |
| | | | | | and 1 hour at 21% |

### 2. Preparation of Test and Control Items

The test and control items were prepared freshly on the day of injection. Lyophilized SHP655 stored at +2 to +8° C. was allowed to reach room temperature. The test article contained rADAMTS13 in the formulation buffer (calcium chloride (2 mM), L-histidine (20 mM), mannitol (3% w/w), sucrose (1% w/w), and polysorbate 80 (0.05% w/w), pH 6.9-7.1). SHP655 was reconstituted in 5 mL of sterile water (sWFI, Lot No. VN549058, Baxalta Innovations GmbH). After reconstitution the test article was kept at room temperature for at least one minute and then gently swirled to ensure complete dissolution. For injection the reconstituted test article was diluted with the formulation buffer for SHP655. The buffer for SHP655 (vehicle) was injected as control. After completion, the formulations were gently mixed by slow inversion. The final dilutions were supplied in a box with wet ice in reasonable labeled tubes (Study No./Group/Dose) filled with the appropriate volume for treatment of the corresponding group. Final dilutions were kept on ice and applied to animals within 3 hours.

### 3. Dosing

The test and control items were injected in conscious restrained animals on one occasion via a lateral tail vein based upon the individual animal's body weights recorded latest a day before injection. Before starting and after completion of the dosing, formulations (100 μL) were stored deep-frozen (<−60° C.).

The day of dosing was designated as day 0. After dosing, the animals were monitored and findings were recorded as given in Section 1 of this Example.

### 4. Hypoxic Studies

The hypoxic studies were conducted using the Biospherix Hypoxia chamber system (OxyCycler Model A84XOV, USA) and according to manufacturer's protocol. Due to the limited capacity of the hypoxic chamber and to facilitate a sound behavioral assessment of the Tim Townes SS mice during the hypoxic challenge the total number of investigated mice per day was limited to 12 individuals. Therefore, the different hypoxic experiments were executed over a time frame of up to three consecutive working days. The impairment of the individual animals focusing on “Mobility” and “Respiratory Rate” parameters was continuously monitored and documented. Animals achieving defined humane endpoints were removed from the hypoxic chamber and euthanized. Although opening and closing of the chamber was accomplished quickly, a transient increase in the oxygen concentration could not be prevented.

About 1 hour after dosing, Tim Townes SS mice were placed in the hypoxic chamber and kept under 7.0% hypoxic conditions for 5 hours followed by 1 hour at 21% oxygen.

After reaching 21% O₂ in the chamber, the software program “Chamber O2 Profile” was stopped and the door of the chamber was opened. During the one-hour recovery phase a skilled operator decided if the individual animal recovered or had to be euthanized due to reaching one of the humane endpoints. The surviving animals were used for a terminal cardiac puncture.

### 5. Behavioral Observations

Upon completion of the recovery phase following exposure to 7.0% hypoxia, comprehensive behavioral observations were performed by a behavioral pharmacologist and a veterinarian. The behavioral symptoms were screened for each mouse individually following the SHIRPA guidelines (Rogers et al., Mamm Genome. 8(10):711-3, 1997) for monitoring disease symptoms as previously described by Irwin (Irwin et al., Psychopharmacologia. 13(3):222-57, 1968). In particular, behavioral items for inspection of the animals during the recovery phase were selected (including: general appearance, posture, spontaneous and induced activity), from which the state of recovery could be estimated. These items were quantitatively scored so that higher numbers were assigned to more severe symptoms (Table 13).

TABLE 13
Behavioral scoring scale

| Symptom | Score Scale | Description |
|---|---|---|
| Piloerection | + | Hair is erected around the body |
| Apathy | + | The mouse appears sleepy |
| Eyes appearance | 0 | Normal |
| | 2 | Half open |
| | 4 | Tightly closed |
| Skin color | + | Jaundice |
| Mobility (spontaneous) | 0 | Normal |
| | 2 | Decreased |
| | 4 | No activity |
| Mobility (stimulated) | 0 | Normal |
| | 2 | Decreased |
| | 4 | No activity |
| Breathing Frequency | 0 | Normal |
| | 1 | Increased |
| | 2 | Deaper |
| | 3 | Decreased |
| | 4 | Irregular |
| Euthanasia | + | Mouse needed to be euthanized |
| Unexpected symptoms | | Describe |

### 6. Blood Sampling

6.1 Cardiac Puncture

At the end of the observation period or after reaching a humane endpoint, a terminal cardiac puncture was performed.

For this purpose animals were anaesthetized (approx. 100-150 mg Ketamin [Lot. No. 6680117, OGRIS Pharma GmbH]+10-20 mg Xylazin [Lot. No. 7630217, OGRIS Pharma GmbH] diluted with NaCl (Lot No. F0718, Medipharm)/kg i.p.) and blood was collected a syringe (2 mL) fitted with a 25G needle without opening the thorax or puncturing the liver. The blood was withdrawn slowly and carefully to prevent circulatory/cardiac collapse. The needle was then removed from the syringe before transferring the sample in a clearly labeled EDTA-tube (250 μL; Lot No. A18033EC, Greiner AG) and into an individually labeled lithium heparin tube (Lot No. 8073011, Sarstedt AG&Co.KG) (remaining blood volume). The tubes were capped and then the samples were gently mixed by slow inversion. The lithium heparin blood was used for plasma preparation (see Section 6.2 of this Example).

6.2 Preparation of Heparin Plasma

All heparinized blood samples were centrifuged as soon as possible. Heparin blood samples were centrifuged at 2200 g for 10 minutes at room temperature. The supernatant plasma was transferred with a plastic pipette into a second clean and clearly labeled Eppendorf tube. Caution was taken to avoid contamination with any cells from the “buffy coat” layer, and samples were transferred into a second labeled Eppendorf tube. A second centrifugation (plasma supernatant) was performed (2200 g for 5 minutes at room temperature). The plasma was again carefully removed with a plastic pipette (no cells from sediment) into a clearly labeled Eppendorf tube. The resulting plasma was subjected to the analyses reported in sections below.

### 7. Analysis of Plasma Samples

Collected mouse samples were used to analyze SHP655 (ADAMTS13) activity and antigen; VWF activity and antigen as well as the level of free-hemoglobin.

7.1 ADAMTS13 Activity Assay (FRETS-VWF73 assay)

The FRETS-VWF73 Assay is a fluorogenic assay measuring the activity of human ADAMTS13.

FRETS-VWF73 is a synthetic fluorogenic peptide consisting of 73 amino acids derived from the VWF A2 domain covering the cleavage site of ADAMTS13 and is used as the minimal peptidyl substrate for the measurement of ADAMTS13 activity. The peptide is modified with two fluorogenic residues (Donor and Acceptor=Quencher). Excitation (λex=340 nm) of uncleaved peptide substrate results in fluorescence resonance energy transfer (FRET) between donor and the vicinal quencher and fluorescence cannot be emitted. Upon cleavage of the peptide substrate by ADAMTS13, no quenching can occur due to the spatial separation of the donor and quencher and fluorescence can be emitted (λem=450 nm) and quantified.

Briefly, samples were diluted (in 100 μL total volume), transferred to a microtiter plate and the reaction was started by addition of the substrate (100 μL FRETS-VWF73; 2 μM final concentration). Fluorescence evolution was measured every two minutes for a period of 60 minutes in a fluorescence spectrophotometer with λex=340 nm and λem=450 nm at 30° C. The increase of fluorescence intensity is proportional to the ADAMTS13 activity concentration in the sample. Samples were measured against a reference standard of diluted pooled normal human plasma (working range from 0.08 to 0.005 U/mL). Human plasma (pooled) from George King Bio-Medical in which the ADAMTS13 concentration is estimated at 1 U/mL, was used as a reference preparation. The resulting FRETS-VWF73 activity data were expressed in U/mL.

7.2 ADAMTS13 Antigen Assay

ADAMTS13 Ag ELISA assay employs the quantitative sandwich enzyme immunoassay technique using in-house (i.e. Baxalta, Orth, Austria) developed anti-ADAMTS13 antibodies. In brief, microtiter plates were coated with polyclonal guinea pig anti-human ADAMTS13 IgG followed by blocking of the non-specific binding sites with blocking solution containing human serum albumin. Test samples, a recombinant standard and quality control samples were then incubated in a total volume of 100 μL per well. After several washing steps, specific binding was detected by the addition of polyclonal rabbit anti-human ADAMTS13 antibody followed by HRP conjugated donkey anti-rabbit IgG and addition of Ultra TMB substrate. The color reaction was stopped by the addition of 1.9 M H2504 and the OD was read at 450 nm and 620 nm (background correction) on a spectrophotometer. The increase in OD at 450 nm was directly proportional to the ADAMTS13 antigen concentration in the sample. Samples were measured against a control preparation of purified rADAMTS13 which was serially diluted and used as reference standard. The reference standard curve was fitted by polynomial regression (2nd order) from which the ADAMTS13 antigen concentration of the test samples is then calculated. ADAMTS13 antigen is expressed in μg/mL.

7.3 VWF Activity Assay

The VWF:CBA was performed according to the product leaflet of ZYMUTEST VWF:CBA (manufactured by Hyphen BioMed, 155, rue d′Eragny, F95000 Neuville-sur-Oise, France) as described in Section 7.1 of Example 2.

7.4 VWF Antigen Assay

The assay was performed according to the product leaflet of ASSERACHROM VWF:Ag (Diagnostica Stago, Asnieres sur Seine, France) as described in Section 7.2 of Example 2.

7.5 VWF Multimer Assay

The multimeric structure of VWF was analyzed by horizontal SDS agarose gel electrophoresis as described in Section 7.3 of Example 2.

7.6 Free Hemoglobin Assay

Free human hemoglobin was analyzed in plasma samples by a commercial sandwich ELISA provided by Abcam (ab157707) as described in Section 7.4 of Example 2.

### 8. Statistical Methods

The statistical analysis was performed with GraphPad PrismVersion 7.03. Data were analyzed using one-way analysis of variance (ANOVA) where differences with a p-value less than 0.05 were considered significant.

### 9. Results

9.1 Body Weight and Age of the Animals

The homozygous Tim Townes SS mice used for the designated investigations were obtained from Jackson Laboratories. Based on the weight monitoring starting at day of delivery (week 0) the animals showed similar gain in average weight and were included at a mean age of 18 to 19 weeks in the exploratory survival studies (see Table 14).

TABLE 14
Mean body weight and age of the animals
at exploratory survival studies

| | | | |
|---|---|---|---|
| Study | Animal | Body weight [g] | Age [weeks] |

| groups | Numbers | Mean | SD | Mean |
|---|---|---|---|---|
| E-H | 21 | 31.6 | 2.0 | 19 |

In the course of the experimental execution, the results of three animals (E37, F44 and H52) revealed unusual phenotypical findings (e.g. aberrant hematological profile). Post-mortem genotyping of the animals demonstrated a heterozygous haplotype. Therefore, the affected animals were excluded from further analyses.

9.2 Experiment at 7.0% Oxygen

The in vivo efficacy of SHP655 was investigated at 7.0% hypoxic conditions. For this purpose, six Tim Townes SS mice per group received 300, 1000 or 3000 U/kg SHP655 one hour before starting the exposure to an O₂ concentration of 7.0%, followed by one-hour recovery phase at 21% O₂ (study groups E-H). The impairment of the individual animals focusing on the assessment parameters “Mobility” and “Respiratory Rate” was continuously monitored and documented by skilled professional during the hypoxic phase. Animals that achieved defined humane endpoints were euthanized. Additionally, subsequent to the 7.0% O₂ hypoxic conditions the behavioral symptoms of the Tim Townes mice which occurred in the course of the recovery phase were scored according to a grading scale based on the SHIRPA guidelines.

Blood samples obtained via terminal cardiac puncture were used for analyses of free hemoglobin, ADAMTS13 and VWF level.

9.2.1 Clinical Symptoms and Mortality

All Tim Townes SS mice revealed treatment independently severe impairment of the assessed “Mobility” and “Respiratory Rate” parameters during the five-hour 7.0% hypoxic phase. One animal treated with 300 IU/kg SHP655 had to be euthanized due to achieving humane (FIG. 11). All other animals survived the observation period of 6 hours. Furthermore, all Tim Townes SS mice investigated showed an improvement of the assessed “Mobility” and “Respiratory Rate” parameters during the one-hour recovery period, with all animals treated with 3000 IU/kg SHP655 recovering completely.

9.2.2 Behavioral Assessment During the Recovery Phase

After the recovery phase, subsequent to the five-hour exposure to 7.0% oxygen a more comprehensive behavioral assessment of the Tim Townes SS mice was conducted. For this purpose the animals were evaluated and scored according to a grading scale based on the SHIRPA guidelines where higher numbers are assigned to more severe symptoms. Piloerection, apathy, breathing frequency and eyes appearance were selected as independent measures of the state of pain/sickness of the animals, given that pain is one of the most common symptoms lamented by patients during sickling crisis (Ballas et al., Blood. 120(18):3647-56, 2012). Spontaneous mobility of the mice was investigated as a surrogate marker of recovery, under the assumption that after several hours under hypoxia mice would feel the need to move around in search for food/water. Likewise, stimulated mobility was evaluated as the spontaneous “flight” reaction.

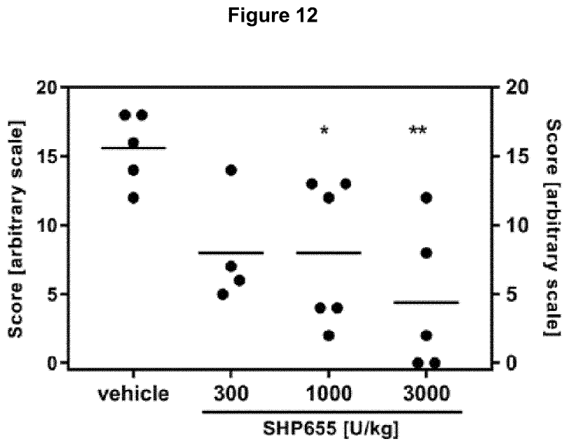

Overall, the use of the behavioral scoring guide allowed for a quantitative measure of the effect of SHP655 on the recovery of the animals from hypoxia (FIG. 12). In particular, SHP655 appeared to demonstrate a dose-dependent effect on the recovery of the animals (300 U/kg p=0.051; 1000 U/kg p<0.05; 3000 U/kg p<0.01).

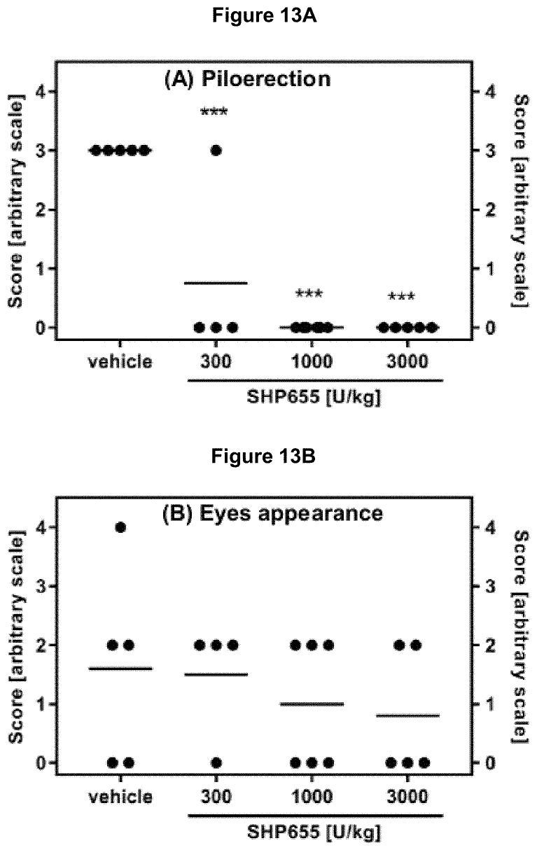

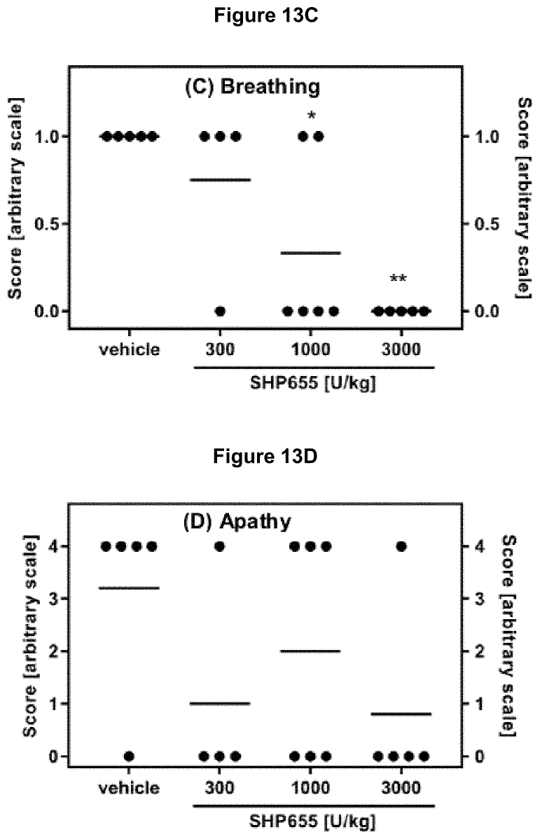

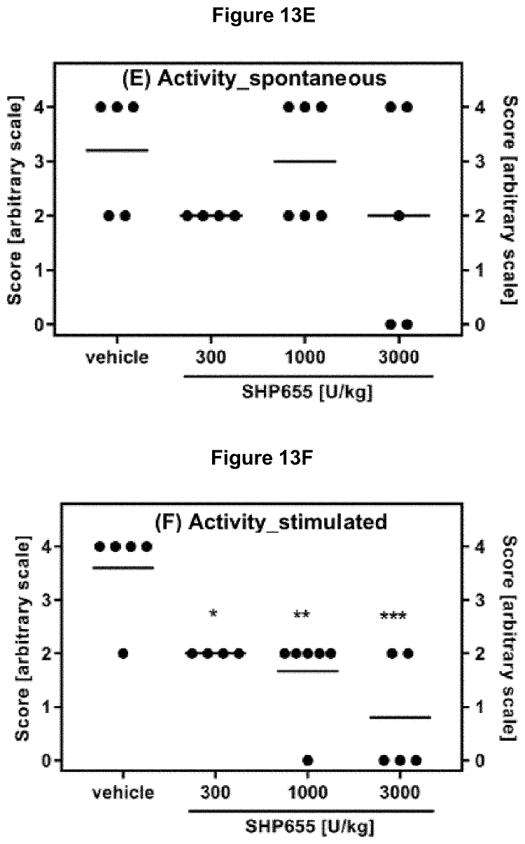

FIGS. 13A-13F summarize the results of single behavioral items where some parameters appeared more indicative of recovery than others. Of all the parameters scored in the tested animals, piloerection (p<0.0001) and stimulated activity (p<0.001) were the best single predictors of recovery from hypoxia (FIGS. 13A and 13F). Additionally, breathing was also significantly improved in mice treated with intermediate (p<0.05) and high (p<0.01) doses of SHP655 (FIG. 13C). Moreover, although no statistically significant differences were measured, apathy and grimace (eyes appearance) were somewhat reduced in SHP655 treated Tim Townes SS mice as compared to vehicle-treated mice. Spontaneous activity as a single end-point observation did not reveal any significant differences, in this study.

9.2.3 Free Hemoglobin in Plasma

Free hemoglobin was analyzed in plasma samples using a commercial ELISA according to manufacturer's instructions.

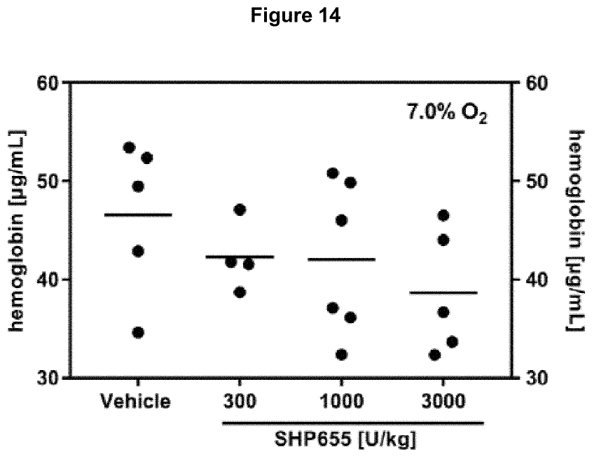

The determination of free hemoglobin levels did not show significant differences between SHP655 treated Tim Townes SS mice and the vehicle group after the 7.0% hypoxic challenge (FIG. 14). However, the mean level of free hemoglobin slightly decreased in a dose-dependent manner.

9.2.4 ADAMTS13 Activity and Antigen

The ADAMTS13 activity and antigen level were determined using the specific FRETS activity assay and an ADAMTS13 ELISA.

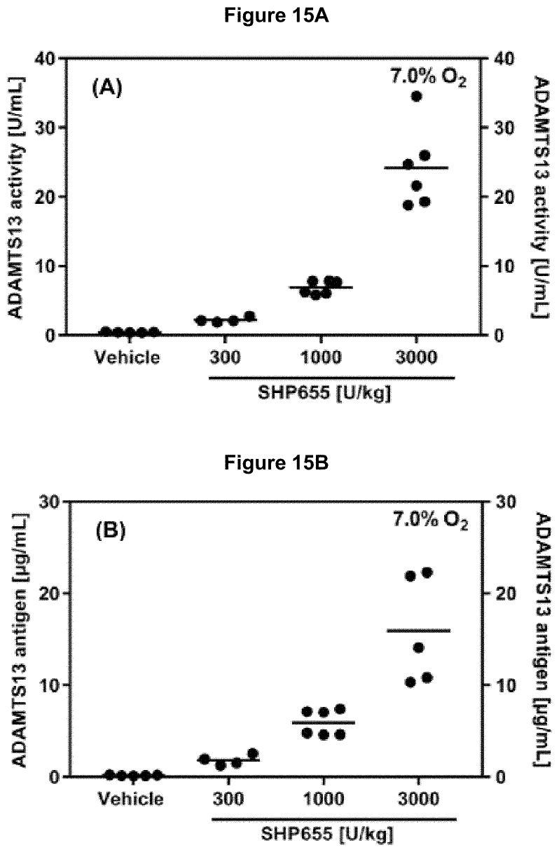

The treatment of the Tim Townes SS mice with SHP655 resulted in a dose-dependent increase of antigen and activity plasma levels of ADAMTS13 (FIGS. 15A-15B). The intermediate and high dose investigated led to mean activity levels that were still significantly different from the vehicle group 6 hours after injection (1000 U/kg p<0.05; 3000 U/kg p<0.001).

9.2.5 VWF Activity and Antigen

VWF activity and antigen level were determined using the ZYMUTEST VWF:CBA activity assay and the ASSERACHROM VWF:Ag ELISA.

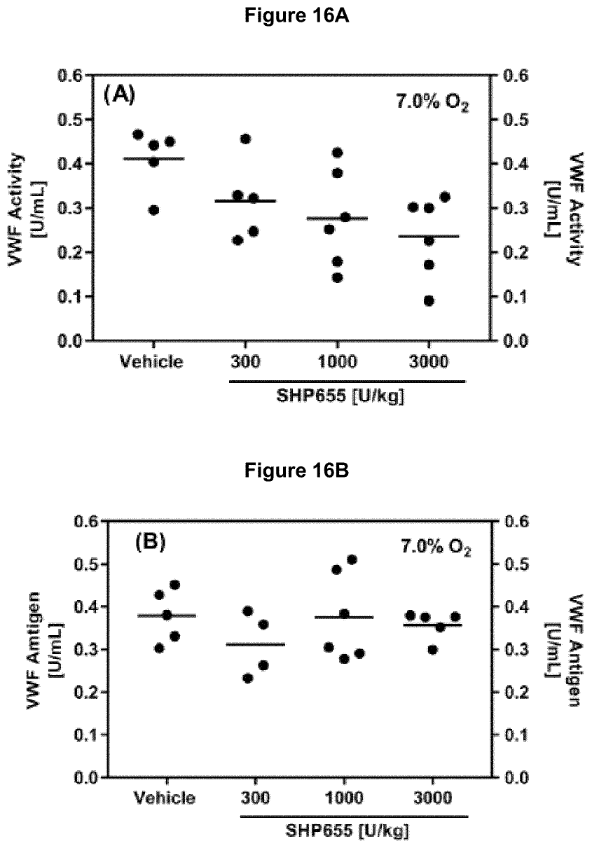

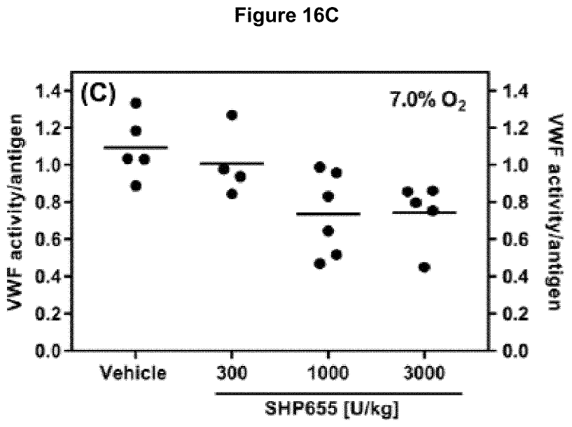

FIGS. 16A-16C displays VWF activity and antigen plasma levels as well as the calculated ratio of the two values. In comparison to the vehicle group, SHP655-treated Tim Townes SS mice showed a significant decrease of VWF activity/antigen ratio at intermediate and high doses (p<0.05), while no differences of VWF total antigen concentration was observed. These results are in line with the proposed mechanism of action of SHP655 in SCD suggesting a decrease of the concentration of ultra-large VWF multimers.

9.2.6 VWF Multimer Analysis

The size distribution of VWF multimers was additionally analyzed by horizontal 1% SDS agarose gel electrophoresis. The samples were diluted based on their VWF:Ag content.

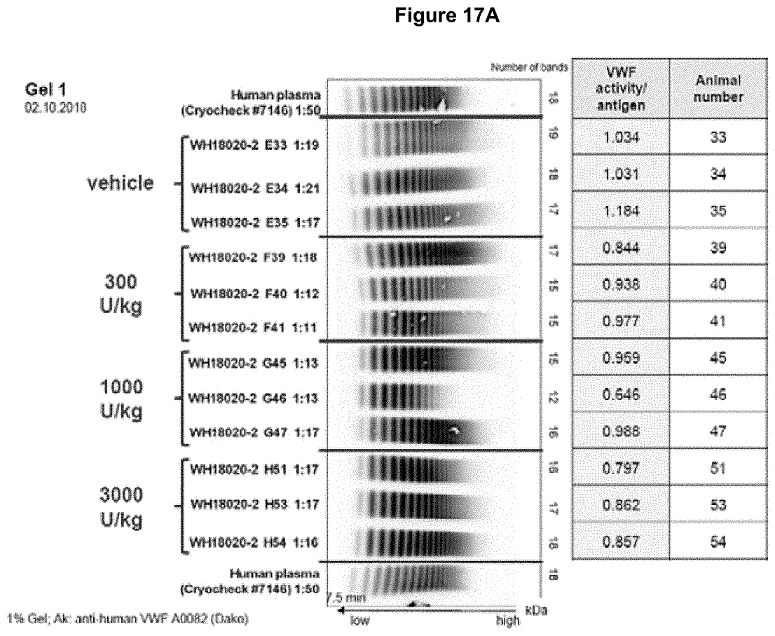

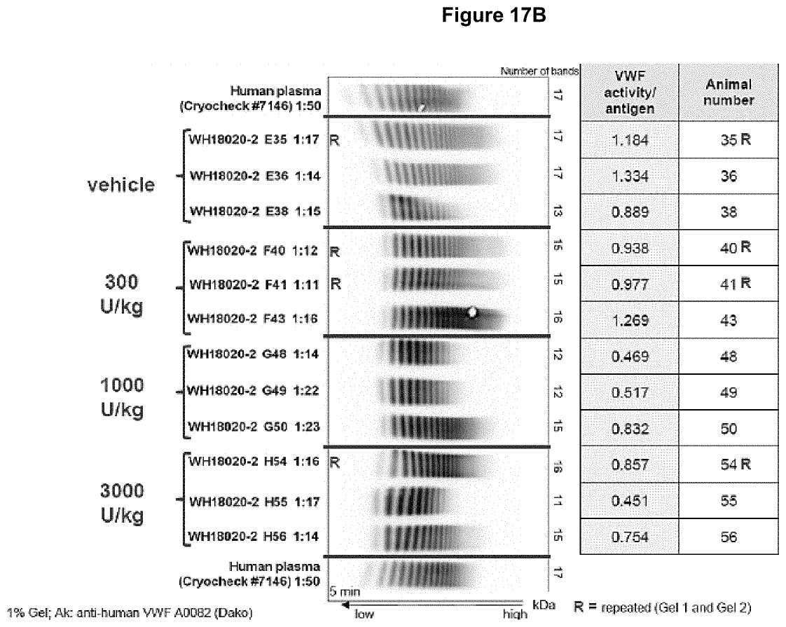

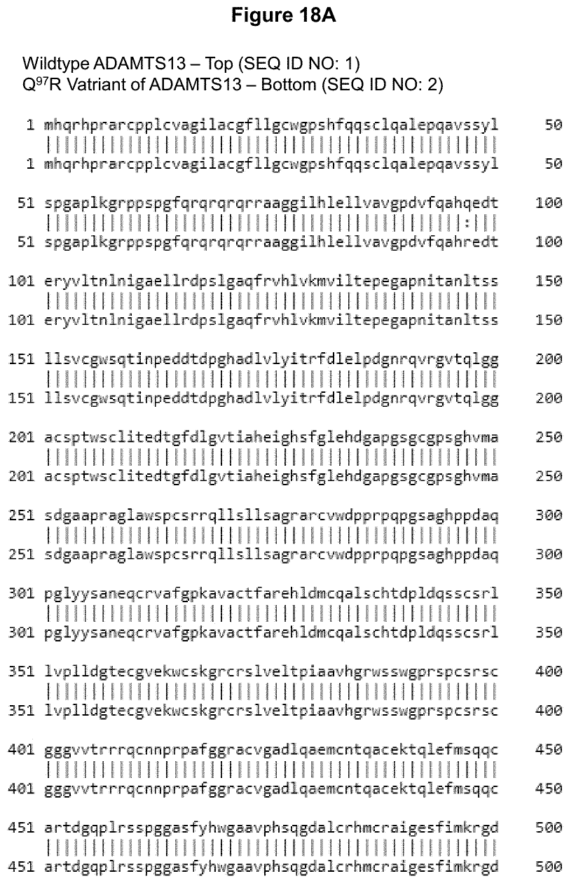

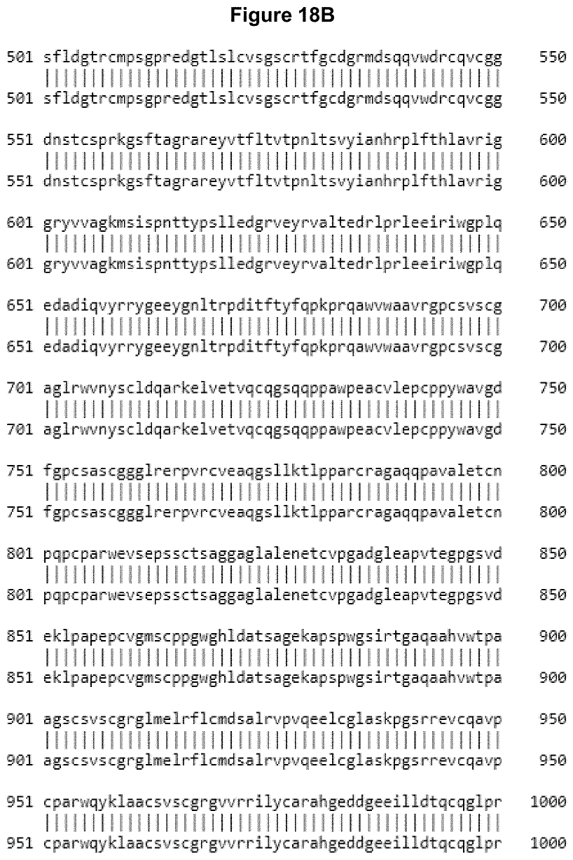

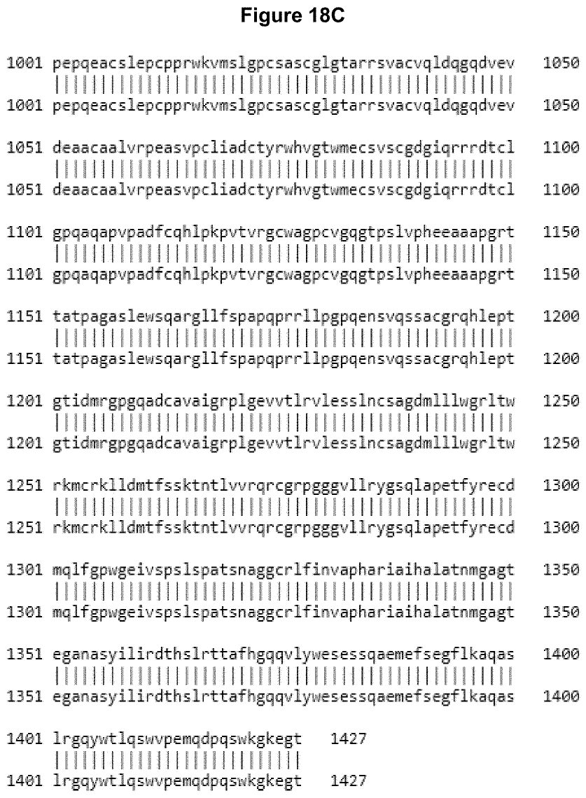

The gels of semi-quantitative VWF multimer analysis of the plasma samples derived from individual Tim Townes SS mice of study groups E to H are displayed in FIGS. 17A-17B. The gel analyses appear to be in accordance to the corresponding VWF activity/antigen values.

### 10. Discussion and Conclusion

All Tim Townes SS mice showed severe impairment of “Mobility” and “Respiratory Rate” in the course of 7.0% O₂ exposure. One animal treated with 300 IU/kg SHP655 had to be euthanized during the five-hour 7.0% hypoxic phase.

All Tim Townes SS mice showed improvement of the assessed “Mobility” and “Respiratory Rate” parameters during the recovery phase after 7.0% hypoxic challenge, where the animals treated with 3000 IU/kg SHP655 recovered completely. The subsequently conducted more comprehensive behavioral scoring according to a grading scale based on the SHIRPA guidelines revealed a significantly improved recovery of the animals treated with 1000 IU/kg (p<0.05) and 3000 IU/kg (p<0.01) SHP655.

A slight, SHP655 dose-dependent decrease of free-hemoglobin level occurred in animals after exposure to 7.0% O₂. The analysis of plasma ADAMTS13 activity and antigen concentration showed that the Tim Townes SS mice were exposed to SHP655 in a dose-dependent manner. The determinations of VWF activity and antigen level in plasma samples obtained from animals of the 7.0% hypoxic approach showed a significant decrease of the VWF activity/antigen ratio at 1000 U/kg (p<0.05) and 3000 IU/kg (p<0.05) SHP655. These findings were confirmed by semi-quantitative VWF multimer analysis demonstrating reduced levels of ultra-large VWF multimers in the plasma of Tim Townes SS mice after treatment with SHP655.

In conclusion, SHP655 significantly improved the recovery of the Tim Townes SS mice after exposure to 7.0% O₂ and decreased the VWF activity/antigen ratio at doses of 1000 IU/kg and 3000 IU/kg, which is in agreement with the proposed mechanism of action of SHP655 in SCD.

This study also suggests that recovery after VOC may also be introduced to inform pharmacologic efficacy studies in mice in SCD. The recovery read-out demonstrated a dose-dependent efficacy of SHP655 in a humanized mouse model of SCD.

The invention has been described in terms of particular embodiments found or proposed to comprise specific modes for the practice of the invention. Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

What is claimed is:

1. A method for increasing A Disintegrin And Metalloproteinase with Thrombospondin type 1 motif, member-13 (ADAMTS13)-mediated VWF cleavage in a subject suffering from sickle cell disease, the method comprising administering to the subject in need thereof a therapeutically effective amount of a composition comprising ADAMTS13.

2. The method of claim 1, wherein the ADAMTS13-mediated VWF cleavage in the subject is inhibited due to an increased plasma level of extracellular hemoglobin (ECHb) compared to a healthy subject.

3. The method of claim 2, wherein the plasma level of extracellular hemoglobin (ECHb) in the subject is about 20-330 μg/mL.

4. The method of claim 2, wherein the plasma level of extracellular hemoglobin (ECHb) in the subject is over 330 μg/mL.

5. The method of any one of claims 1-4, wherein administering ADAMTS13 results in a reduction in the levels of at least one of ultra-large VWF multimers, VWF activity and VWF activity/antigen ratio compared to without ADAMTS13 treatment.

6. The method of any one of claims 1-5, wherein administering ADAMTS13 results in a reduction in the level of free hemoglobin in the plasma compared to without ADAMTS13 treatment.

7. A method for treating a vaso-occlusive crisis (VOC) in a subject suffering from sickle cell disease, the method comprising administering to the subject in need thereof a therapeutically effective amount of a composition comprising ADAMTS13 after the onset of the VOC.

8. A method for preventing a vaso-occlusive crisis (VOC) in a subject suffering from sickle cell disease, the method comprising administering to the subject in need thereof a therapeutically effective amount of a composition comprising ADAMTS13 prior to the onset of the VOC.

9. The method of any one of claim 1-8, wherein the composition further comprises an ADAMTS13 variant.

10. The method of claim 9, wherein the ADAMTS13 variant comprises an amino acid sequence with at least one single amino acid substitution as compared to the wildtype ADAMT13.

11. The method of claim 10, wherein the wildtype ADAMTS13 is a human ADAMTS13.

12. The method of claim 10, wherein the wildtype ADAMTS13 comprises the amino acid sequence of SEQ ID NO: 1.

13. The method of any of claims 9-12, wherein at least one of the single amino acid substitutions is within the ADAMTS13 catalytic domain as compared to wildtype ADAMTS13.

14. The method of claim 13, wherein the single amino acid substitution is not I⁷⁹M, V⁸⁸M, H⁹⁶D, R¹⁰²C, S¹¹⁹F, I¹⁷⁸T, R¹⁹³W, T196I, S²⁰³P, L²³²Q, H²³⁴Q, D²³⁵H, A²⁵⁰V, S²⁶³C, and/or R²⁶⁸P as denoted in SEQ ID NO: 1, or the equivalent amino acid in an ADAMTS13.

15. The method of any of claims 9-14, wherein the single amino acid substitution is at amino acid Q⁹⁷ as denoted in SEQ ID NO: 1, or the equivalent amino acid in an ADAMTS13.

16. The method of claim 15, wherein the single amino acid change is from a Q to a D, E, K, H, L, N, P, or R.

17. The method of claim 15 or 16, wherein the single amino acid change is from a Q to an R.

18. The method of any one of claims 9-17, wherein the ADAMTS13 variant comprises the amino acid sequence of SEQ ID NO: 2.

19. The method of any one of claims 9-18, wherein the ADAMTS13 variant consists essentially of SEQ ID NO: 2.

20. The method of any one of claims 10-20, wherein the ADAMTS13 variant consists of SEQ ID NO: 2.

21. The method of any one of claims 1-20, wherein the therapeutically effective amount of ADAMTS13 and/or a variant thereof is from about 20 to about 6,000 international units per kilogram body weight.

22. The method of any one of claims 1-21, wherein the therapeutically effective amount of ADAMTS13 and/or a variant thereof is from about 300 to about 3,000 international units per kilogram body weight.

23. The method of any one of claims 1-22, wherein the therapeutically effective amount of ADAMTS13 and/or a variant thereof is from about 1000 to about 3,000 international units per kilogram body weight.

24. The method of any one of claims 1-23, wherein administering the therapeutically effective amount of ADAMTS13 and/or a variant thereof results in a plasma concentration of ADAMTS13 and/or a variant thereof at about 1 to about 80 U/mL in the subject.

25. The method of any one of claims 1-24, wherein the composition comprising ADAMTS13 and/or a variant thereof is administered in a single bolus injection, monthly, every two weeks, weekly, twice a week, daily, every 12 hours, every eight hours, every six hours, every four hours, or every two hours.

26. The method of any one of claims 1-25, wherein the composition comprising ADAMTS13 and/or a variant thereof is administered intravenously or subcutaneously.

27. The method of any one of claims 1-26, wherein the ADAMTS13 and/or a variant thereof is recombinant.

28. The method of any one of claims 1-27, wherein the ADAMTS13 and/or a variant thereof is plasma derived.

29. The method of any one of claims 1-28, wherein the composition is in a stable aqueous solution ready for administration.

30. The method of any one of claims 1-29, wherein the therapeutically effective amount of the composition comprising ADAMTS13 and/or a variant thereof is sufficient to maintain an effective level of ADAMTS13 activity in the subject.

31. The method of any one of claims 1-30, wherein the subject is a mammal.

32. The method of any one of claims 1-30, wherein the subject is a human.

33. A method of determining the efficacy of a treatment for a vaso-occlusive crisis (VOC) in a subject, said method comprising:
a) applying the treatment to the subject after the VOC;
b) collecting from the subject one or more behavioral symptoms selected from piloerection, apathy, eyes appearance, skin color, spontaneous mobility, stimulated mobility, and breathing frequency;
c) generating a score based on the severity of the one or more behavioral symptoms collected from step b);
d) comparing the score from step c) to a control score, wherein the control score is generated from a control subject that does not receive a treatment; and
e) (i) determining the treatment is effective if the score from step c) indicates less severity compared to the control score; (ii) determining the treatment is not effective if the score from step c) indicates more or the same severity compared to the control score.

34. A method of assessing the recovery of a subject from a vaso-occlusive crisis (VOC), said method comprising:
a) collecting from the subject one or more behavioral symptoms selected from piloerection, apathy, eyes appearance, skin color, spontaneous mobility, stimulated mobility, and breathing frequency after the VOC;
b) generating a score based on the severity of the one or more behavioral symptoms collected from step a);
c) comparing the score from step b) to a control score, wherein the control score is generated from the subject before the VOC or from a control subject that does not have a VOC; and
d) (i) determining the subject has recovered if the score from step b) indicates less or the same severity compared to the control score; (ii) determining the subject has not recovered if the score from step b) indicates more severity compared to the control score.

35. The method of claim 33 or claim 34, wherein the one or more behavioral symptoms are selected from piloerection, apathy, eyes appearance, stimulated mobility, and breathing frequency.

36. The method of any one of claims 33-35, wherein the behavioral symptoms are scored such that higher numbers are assigned to more severe symptoms.

37. The method of any one of claims 33-36, wherein the subject is a mammal.

38. The method of any one of claims 33-37, wherein the subject is a mouse.
